SEQUENCE LISTING (40 sequences)

SEQ ID NO: 1 - protein, 176 aa, Glycine max
MPRSTDPLVIGGVIGDVLEPFTSSVSMGIVYNNCPQVINCCELKPSKILNRPRIEIGGDDLRTFYTLVMVDPDAPSPGNPTQREYLHWLITNIPATTGANFGEEIVSYESPRPIVGIHRIVFVLFRQLRRLTLQPPGWRQNFNTRDFAEIYNLGLPVAAMYFNCKRENDQSSGRRR

SEQ ID NO: 2 - protein, 141 aa, Glycine max
MPRSTDPLVIGGVIGDVLEPFTSSVSMGIVYNNCPQVINCCELKPSKILNRPRIEIGGDDLRTFYTLVMVDPDAPSPGNPTQREYLHWLITNIPATTGANFGWRQNFNTRDFAEIYNLGLPVAAMYFNCKRENDQSSGRRR

SEQ ID NO: 3 - DNA, 5737 bp, Glycine max
  source 1..5737: mol_type = genomic DNA
atcctgcaactgtctgagagattttttttccctcacagcgttttctctccattttaacaattatagaaataaatctaagaagtaaagtaccggaatctactagatgttgaaatattaatatgagtaaacaaacgctagcttagtgcatgtataaataggcatagcggcatagacaattttccctcataaaccgatagaaggtgaccaacttatcgatattaatttattatgcctagatcaacggaccctcttgttattggaggtgtaataggagatgttttggagcctttcacaagtagcgtttctatggggatagtctataataattgccctcaagttatcaactgctgtgagctcaaaccctccaaaatcctcaatcgaccaagaattgaaattggtggagatgacctcaggaccttttacaccctggtattatgtaattatgtgttaattgcctttaacttatattatatcttcattttaattgatttattatatatcgaattatctacatacgtacgtttctgtcactctttagggtcgttaataattatggttgattttattgcatgcatggcacttacataatttgcaggttatggtggatcctgatgcacctagcccaggcaatccaacccagagagaatatttgcactggtaattaattaagtatccctccctcgcttccatgatctgctccaccttgatgaaataagtcatcgaaactatacatatatattcacgtacggatgcacgtacgtatttaattacgagttcacttatcttcgatgaattgacttgtgtataaattctctttttaatagactttataaactttaagtaaattcgttcgattcttgagtttattattaactcagtaagttaaaaaaattcaaccaaaatataagtcattctgaattattttctgtctacattcatttagtatgttattttctgataaaataattatcacctttaataatattaaacacatgttctctaataacgttaaactttggatggaaataatctaccacactattataatatctgcacgttattatctagtaatgatatattattagacttggttatttaagtattttataatttataattttttttattattatattgttgaaatatttaattgatatattatttataaatattttattattatttttaaatgaaatagattcttacgaatttataaattggatacaggaggtaagtctataaaattctaaaaaatttacataatctcttcagtttgataatcttttatatatataaaaacatatgatatatggccttgcagtttttagatatattgaagtttatggcaatagggtttataaaaattaatctagatgccttttttatccaccatcatttctctccaaatcaatttaaacttcctcttagagtggtgttaatgtttacgtgagattgcatatatcttgatagatagtctatacttctagcatctacttttcattgactccaaatatatattctgaacttttctcataccttttctctattctatcttttggtaaacatatactctatcatacttagatgctttttaaactttgcatccatatctttttactttctgaaaaatactctgatttttttcaataacaggtataaaaagtacttgtttaaatatttttttttcttccttttcttttttatttggcccctatatgtgaaaaaaaagtttgtaaacgaatctctttttttatttttactcagcaaaaatatgtaactatatttcaggggcacaatgggtgcccaaaccatttacaagaaaggaaataatcctaacatggtagctaaaactcccacggttacacaatattaacatgtaaacgagtctcttgattaagaaaatgtttttacgtagtgtttcctattataatctcatcttataatgctattagatcaagtctgacctgactctaatagatttttaacgtagcttctaacacgtgagaaagctttatacatgttttcattatgcatgatttctatacgtataacacactttgtttcatttaggttcatatatatatatatatatatatatatatatatatatatatatatattgttcaaaaataaaagttctataaaaaaatgtattatcgtcttaactcggatattatttttacaaaagcagcgcccgcttgcgtaagtacttattatgtttagttggtatcagttttgtcaacgctgaaagtacaacaatgaatagcacggagcaaactagttgatatatttcaagagaatcttttgttcaagagaatctttagttatagattattaacatgttcataaaaaaaagaaaagtgaatatcttaataataataaaacattggttaacatatacgtattcatgttttggtgtaaatgtgttaataaactacatgatggatatattttaagataatatttaatatttataatttgttatgcactcgtactaaaaaataaaaatgtgtatattaacaaactcacatatatatatatatatatatataaaagtttgtcaatatacatatatatattttttagtataaatgcgttaataaattatattattgatatattttaagatatattttaattataatttattaatacattcatataaaaaaaataacagtgtttatgttaaaaattcataaataattattaaataaactcacatatatatatatatatatatatatatatatatatatatatatatatatatatatatatatatatatgatatgttatttaatcttctcacctcttataataaaatattcttctcacttgatatatatatatatgacgggtcaagttgaattataaattcaaaaataatatccataattacttattaattattggatttgaacatattgaattaaatttaatccaaatcctcaaaatccaaacccatattaattgggttgatttaaatttataaatgatccatataagtaaacacatttttacttgtgaatcacttttttcttctttctttctatcttttcaaggatattggttgttcaagaataattttacttatgggataataagaaaatagatgttgtggaatgttcaaataaataactatttaggtattattatttctcataaaatatctcatccacgcggatcaacttttaattttctaaatttaaactactaacatgaaataatgttatcttaaagaaaatataaaatattagagaaaattaccataatacctcctgagctttttattaaattacatcttacacataattttatttatttatttattattttacactgattttctttaaatttgaaaatattgtactaacattcggttatatgtttgaaaacattatattgatattttgaaaacattataatgatattctttaatatttctacaaattttatactaacacaccttaccaaagagatgaatataataattgaaaaagtaagtatatttgtgtaattacacgaaatctcagaaattattgttataatttgctcaaaattttatattggacgccactttgcttaaatatattttattaacaaattacgaattgaaattctttttcattgatgaatgaataggttgataaccaatattcctgcaactacaggggcaaacttcggtatgtgcacaattcttattttctatatatcttccttgtacaccttttatatttaattggcagtaaattataaagccttcgtgttattatgtctgatcaaagtaaatctcaaatttctttaaattgatattactttcaatcagttataaaacatcttattaattgtatcagtgttttaaatttagaatataaattaaaaatttatgaagtatatttcaaattataataaaatatattgtttgtttaggtagaaatgtagaatgtaaaaatatatattaaatagaggaaatatttaaaataataaaaactatacgaataaataattcaacaaaatatattgaatttaaaatttaaaataagagagctaaaagttttaatggagagtataaaaaaaagaaagtgaactttattttttcatataagggtctattttaaatttatataaaagtttcgaaatgataacaaaaagttcccaatttataatttgtgattctattttatgaattatattttaagatatcataaaaaaataaaaagtatttgaataagaaactctttaaaaaataaatttaattagtgaaatatttaattagttgcaattatttaaatctttaatttatttttcaaattataaatcagaataatatatcattgaattattgatttacattttataatgattttcttaaaattgtagcataaattatttcaaacattttaacagaatacacaagtattttgagttaatatctatacaaaaaaattattataataattaagaaatttaaatttcatgagcatgattcaaatgacccaaatttttaatccaatttttaattatataagatattcattttcttagaaatgatttacttgtgtgcatatatatcctcgactattcatatcatcaagtgattagttcaccttgctacaggaatgtttaatttgaagaaaaaaaactcaaattcgaattgtttgtatggtaaaaccttttgttgagagagttaatttcgtatgtgtgtttctttctcaaacataataggttatagtggaggatatgtgtgaatgtgatttagataaaagaaaatatgtccatcattattttaattataaaccatattaaatctcgtaagaaatttattaattcgaatgagtatatgataattatttatattttaagatataaattgcatgtgagaaatagtaaatattttgattaagctataaaatttgtgtcatgctgtaatagactgtctttgaactgttatcaatatatgtggatttatttattacttcaataaagggtaatttacaggagaagagattgtctcctatgaaagtccacgtccaatagtagggattcatcgaatagtttttgtgttatttcgtcagctgcgtagactaactctgcaacctccaggctggcgccagaatttcaacactagagactttgctgagatttataatcttggattaccagtagcggccatgtacttcaactgtaaacgagaaaatgatcaaagcagtggaagaagaagataatagaaagaaaactattattaatatatatcccgagtttttatcagctcaacatgaaataataaataacttctttttatatttaagtatttattaaatataaatatatattatataaaaaaatcaacacgaatttcaattaaaaaataatattagtatgaaagagctaatggcccctaaagaaaaaggtgcctagggcatcagcaataagcttagtatgaaggtcatttggacatacctgccaaactgaagatcttcagacgagccattaatttcccagcaaggtaatctgcaatctgattagcttctctgaatgtgtgttggaacaccagattccactcaagctgcatgaaggttctgatcttggtaatcaaaggagacacattcacaaataattatgtacgtatgaggaaataatcgattaatctacttgttcttcaaactcatatttatgaagagcaaagtaattgattaattctgttgattttataaataacatctttgttttttcattg

SEQ ID NO: 4 - DNA, 287 bp, synthetic construct
  source 1..287: mol_type = other DNA
gcttagtgcatgtataaataggcatagcggcatagacaattttccctcataaaccgatagaaggtgaccaacttatcgatattaatttattatgcctagatcaacggaccctcttgttattggaggtgtaataggagatgttttggagcctttcacaagtagcgtttctatggggatagtctataataattgccctcaagtgagctcaaaccctccaaaatcctcaatcgaccaagaattgaaattggtggagatgacctcaggaccttttacaccctggtattatg

SEQ ID NO: 5 - DNA, 5724 bp, synthetic construct
  source 1..5724: mol_type = other DNA
atcctgcaactgtctgagagattttttttccctcacagcgttttctctccattttaacaattatagaaataaatctaagaagtaaagtaccggaatctactagatgttgaaatattaatatgagtaaacaaacgctagcttagtgcatgtataaataggcatagcggcatagacaattttccctcataaaccgatagaaggtgaccaacttatcgatattaatttattatgcctagatcaacggaccctcttgttattggaggtgtaataggagatgttttggagcctttcacaagtagcgtttctatggggatagtctataataattgccctcaagtgagctcaaaccctccaaaatcctcaatcgaccaagaattgaaattggtggagatgacctcaggaccttttacaccctggtattatgtaattatgtgttaattgcctttaacttatattatatcttcattttaattgatttattatatatcgaattatctacatacgtacgtttctgtcactctttagggtcgttaataattatggttgattttattgcatgcatggcacttacataatttgcaggttatggtggatcctgatgcacctagcccaggcaatccaacccagagagaatatttgcactggtaattaattaagtatccctccctcgcttccatgatctgctccaccttgatgaaataagtcatcgaaactatacatatatattcacgtacggatgcacgtacgtatttaattacgagttcacttatcttcgatgaattgacttgtgtataaattctctttttaatagactttataaactttaagtaaattcgttcgattcttgagtttattattaactcagtaagttaaaaaaattcaaccaaaatataagtcattctgaattattttctgtctacattcatttagtatgttattttctgataaaataattatcacctttaataatattaaacacatgttctctaataacgttaaactttggatggaaataatctaccacactattataatatctgcacgttattatctagtaatgatatattattagacttggttatttaagtattttataatttataattttttttattattatattgttgaaatatttaattgatatattatttataaatattttattattatttttaaatgaaatagattcttacgaatttataaattggatacaggaggtaagtctataaaattctaaaaaatttacataatctcttcagtttgataatcttttatatatataaaaacatatgatatatggccttgcagtttttagatatattgaagtttatggcaatagggtttataaaaattaatctagatgccttttttatccaccatcatttctctccaaatcaatttaaacttcctcttagagtggtgttaatgtttacgtgagattgcatatatcttgatagatagtctatacttctagcatctacttttcattgactccaaatatatattctgaacttttctcataccttttctctattctatcttttggtaaacatatactctatcatacttagatgctttttaaactttgcatccatatctttttactttctgaaaaatactctgatttttttcaataacaggtataaaaagtacttgtttaaatatttttttttcttccttttcttttttatttggcccctatatgtgaaaaaaaagtttgtaaacgaatctctttttttatttttactcagcaaaaatatgtaactatatttcaggggcacaatgggtgcccaaaccatttacaagaaaggaaataatcctaacatggtagctaaaactcccacggttacacaatattaacatgtaaacgagtctcttgattaagaaaatgtttttacgtagtgtttcctattataatctcatcttataatgctattagatcaagtctgacctgactctaatagatttttaacgtagcttctaacacgtgagaaagctttatacatgttttcattatgcatgatttctatacgtataacacactttgtttcatttaggttcatatatatatatatatatatatatatatatatatatatatatatattgttcaaaaataaaagttctataaaaaaatgtattatcgtcttaactcggatattatttttacaaaagcagcgcccgcttgcgtaagtacttattatgtttagttggtatcagttttgtcaacgctgaaagtacaacaatgaatagcacggagcaaactagttgatatatttcaagagaatcttttgttcaagagaatctttagttatagattattaacatgttcataaaaaaaagaaaagtgaatatcttaataataataaaacattggttaacatatacgtattcatgttttggtgtaaatgtgttaataaactacatgatggatatattttaagataatatttaatatttataatttgttatgcactcgtactaaaaaataaaaatgtgtatattaacaaactcacatatatatatatatatatatataaaagtttgtcaatatacatatatatattttttagtataaatgcgttaataaattatattattgatatattttaagatatattttaattataatttattaatacattcatataaaaaaaataacagtgtttatgttaaaaattcataaataattattaaataaactcacatatatatatatatatatatatatatatatatatatatatatatatatatatatatatatatatatgatatgttatttaatcttctcacctcttataataaaatattcttctcacttgatatatatatatatgacgggtcaagttgaattataaattcaaaaataatatccataattacttattaattattggatttgaacatattgaattaaatttaatccaaatcctcaaaatccaaacccatattaattgggttgatttaaatttataaatgatccatataagtaaacacatttttacttgtgaatcacttttttcttctttctttctatcttttcaaggatattggttgttcaagaataattttacttatgggataataagaaaatagatgttgtggaatgttcaaataaataactatttaggtattattatttctcataaaatatctcatccacgcggatcaacttttaattttctaaatttaaactactaacatgaaataatgttatcttaaagaaaatataaaatattagagaaaattaccataatacctcctgagctttttattaaattacatcttacacataattttatttatttatttattattttacactgattttctttaaatttgaaaatattgtactaacattcggttatatgtttgaaaacattatattgatattttgaaaacattataatgatattctttaatatttctacaaattttatactaacacaccttaccaaagagatgaatataataattgaaaaagtaagtatatttgtgtaattacacgaaatctcagaaattattgttataatttgctcaaaattttatattggacgccactttgcttaaatatattttattaacaaattacgaattgaaattctttttcattgatgaatgaataggttgataaccaatattcctgcaactacaggggcaaacttcggtatgtgcacaattcttattttctatatatcttccttgtacaccttttatatttaattggcagtaaattataaagccttcgtgttattatgtctgatcaaagtaaatctcaaatttctttaaattgatattactttcaatcagttataaaacatcttattaattgtatcagtgttttaaatttagaatataaattaaaaatttatgaagtatatttcaaattataataaaatatattgtttgtttaggtagaaatgtagaatgtaaaaatatatattaaatagaggaaatatttaaaataataaaaactatacgaataaataattcaacaaaatatattgaatttaaaatttaaaataagagagctaaaagttttaatggagagtataaaaaaaagaaagtgaactttattttttcatataagggtctattttaaatttatataaaagtttcgaaatgataacaaaaagttcccaatttataatttgtgattctattttatgaattatattttaagatatcataaaaaaataaaaagtatttgaataagaaactctttaaaaaataaatttaattagtgaaatatttaattagttgcaattatttaaatctttaatttatttttcaaattataaatcagaataatatatcattgaattattgatttacattttataatgattttcttaaaattgtagcataaattatttcaaacattttaacagaatacacaagtattttgagttaatatctatacaaaaaaattattataataattaagaaatttaaatttcatgagcatgattcaaatgacccaaatttttaatccaatttttaattatataagatattcattttcttagaaatgatttacttgtgtgcatatatatcctcgactattcatatcatcaagtgattagttcaccttgctacaggaatgtttaatttgaagaaaaaaaactcaaattcgaattgtttgtatggtaaaaccttttgttgagagagttaatttcgtatgtgtgtttctttctcaaacataataggttatagtggaggatatgtgtgaatgtgatttagataaaagaaaatatgtccatcattattttaattataaaccatattaaatctcgtaagaaatttattaattcgaatgagtatatgataattatttatattttaagatataaattgcatgtgagaaatagtaaatattttgattaagctataaaatttgtgtcatgctgtaatagactgtctttgaactgttatcaatatatgtggatttatttattacttcaataaagggtaatttacaggagaagagattgtctcctatgaaagtccacgtccaatagtagggattcatcgaatagtttttgtgttatttcgtcagctgcgtagactaactctgcaacctccaggctggcgccagaatttcaacactagagactttgctgagatttataatcttggattaccagtagcggccatgtacttcaactgtaaacgagaaaatgatcaaagcagtggaagaagaagataatagaaagaaaactattattaatatatatcccgagtttttatcagctcaacatgaaataataaataacttctttttatatttaagtatttattaaatataaatatatattatataaaaaaatcaacacgaatttcaattaaaaaataatattagtatgaaagagctaatggcccctaaagaaaaaggtgcctagggcatcagcaataagcttagtatgaaggtcatttggacatacctgccaaactgaagatcttcagacgagccattaatttcccagcaaggtaatctgcaatctgattagcttctctgaatgtgtgttggaacaccagattccactcaagctgcatgaaggttctgatcttggtaatcaaaggagacacattcacaaataattatgtacgtatgaggaaataatcgattaatctacttgttcttcaaactcatatttatgaagagcaaagtaattgattaattctgttgattttataaataacatctttgttttttcattg

SEQ ID NO: 6 - protein, 84 aa, synthetic construct
MPRSTDPLVIGGVIGDVLEPFTSSVSMGIVYNNCPQVSSNPPKSSIDQELKLVEMTSGPFTPWLWWILMHLAQAIQPRENICTG

SEQ ID NO: 7 - DNA, 291 bp, synthetic construct
  source 1..291: mol_type = other DNA
gcttagtgcatgtataaataggcatagcggcatagacaattttccctcataaaccgatagaaggtgaccaacttatcgatattaatttattatgcctagatcaacggaccctcttgttattggaggtgtaataggagatgttttggagcctttcacaagtagcgtttctatggggatagtctataataattgccctcaagttatcgagctcaaaccctccaaaatcctcaatcgaccaagaattgaaattggtggagatgacctcaggaccttttacaccctggtattatg

SEQ ID NO: 8 - DNA, 2935 bp, synthetic construct
  source 1..2935: mol_type = other DNA
atcctgcaactgtctgagagattttttttccctcacagcgttttctctccattttaacaattatagaaataaatctaagaagtaaagtaccggaatctactagatgttgaaatattaatatgagtaaacaaacgctagcttagtgcatgtataaataggcatagcggcatagacaattttccctcataaaccgatagaaggtgaccaacttatcgatattaatttattatgcctagatcaacggaccctcttgttattggaggtgtaataggagatgttttggagcctttcacaagtagcgtttctatggggatagtctataataattgccctcaagttatcgagctcaaaccctccaaaatcctcaatcgaccaagaattgaaattggtggagatgacctcaggaccttttacaccctggtattatgtaattatgtgttaattgcctttaacttatattatatcttcattttaattgatttattatatatcgaattatctacatacgtacgtttctgtcactctttagggtcgttaataattatggttgattttattgcatgcatggcacttacataatttgcaggttatggtggatcctgatgcacctagcccaggcaatccaacccagagagaatatttgcactggtaattaattaagtatccctccctcgcttccatgatctgctccaccttgatgaaataagtcatcgaaactatacatatatattcacgtacggatgcacgtacgtatttaattacgagttcacttatcttcgatgaattgacttgtgtataaattctctttttaatagactttataaactttaagtaaattcgttcgattcttgagtttattattaactcagtaagttaaaaaaattcaaccaaaatataagtcattctgaattattttctgtctacattcatttagtatgttattttctgataaaataattatcacctttaataatattaaacacatgttctctaataacgttaaactttggatggaaataatctaccacactattataatatctgcacgttattatctagtaatgatatattattagacttggttatttaagtattttataatttataattttttttattattatattgttgaaatatttaattgatatattatttataaatattttattattatttttaaatgaaatagattcttacgaatttataaattggatacaggaggtaagtctataaaattctaaaaaatttacataatctcttcagtttgataatcttttatatatataaaaacatatgatatatggccttgcagtttttagatatattgaagtttatggcaatagggtttataaaaattaatctagatgccttttttatccaccatcatttctctccaaatcaatttaaacttcctcttagagtggtgttaatgtttacgtgagattgcatatatcttgatagatagtctatacttctagcatctacttttcattgactccaaatatatattctgaacttttctcataccttttctctattctatcttttggtaaacatatactctatcatacttagatgctttttaaactttgcatccatatctttttactttctgaaaaatactctgatttttttcaataacaggtataaaaagtacttgtttaaatatttttttttcttccttttcttttttatttggcccctatatgtgaaaaaaaagtttgtaaacgaatctctttttttatttttactcagcaaaaatatgtaactatatttcaggggcacaatgggtgcccaaaccatttacaagaaaggaaataatcctaacatggtagctaaaactcccacggttacacaatattaacatgtaaacgagtctcttgattaagaaaatgtttttacgtagtgtttcctattataatctcatcttataatgctattagatcaagtctgacctgactctaatagatttttaacgtagcttctaacacgtgagaaagctttatacatgttttcattatgcatgatttctatacgtataacacactttgtttcatttaggttcatatatatatatatatatatatatatatatatatatatatatatattgttcaaaaataaaagttctataaaaaaatgtattatcgtcttaactcggatattatttttacaaaagcagcgcccgcttgcgtaagtacttattatgtttagttggtatcagttttgtcaacgctgaaagtacaacaatgaatagcacggagcaaactagttgatatatttcaagagaatcttttgttcaagagaatctttagttatagattattaacatgttcataaaaaaaagaaaagtgaatatcttaataataataaaacattggttaacatatacgtattcatgttttggtgtaaatgtgttaataaactacatgatggatatattttaagataatatttaatatttataatttgttatgcactcgtactaaaaaataaaaatgtgtatattaacaaactcacatatatatatatatatatatataaaagtttgtcaatatacatatatatattttttagtataaatgcgttaataaattatattattgatatattttaagatatattttaattataatttattaatacattcatataaaaaaaataacagtgtttatgttaaaaattcataaataattattaaataaactcacatatatatatatatatatatatatatatatatatatatatatatatatatatatatatatatatatgatatgttatttaatcttctcacctcttataataaaatattcttctcacttgatatatatatatatgacgggtcaagttgaattataaattcaaaaataatatccataattacttattaattattggatttgaacatattgaattaaatttaatccaa

SEQ ID NO: 9 - protein, 173 aa, synthetic construct
MPRSTDPLVIGGVIGDVLEPFTSSVSMGIVYNNCPQVIELKPSKILNRPRIEIGGDDLRTFYTLVMVDPDAPSPGNPTQREYLHWLITNIPATTGANFGEEIVSYESPRPIVGIHRIVFVLFRQLRRLTLQPPGWRQNFNTRDFAEIYNLGLPVAAMYFNCKRENDQSSGRRR

SEQ ID NO: 10 - protein, 138 aa, synthetic construct
MPRSTDPLVIGGVIGDVLEPFTSSVSMGIVYNNCPQVIELKPSKILNRPRIEIGGDDLRTFYTLVMVDPDAPSPGNPTQREYLHWLITNIPATTGANFGWRQNFNTRDFAEIYNLGLPVAAMYFNCKRENDQSSGRRR

SEQ ID NO: 11 - DNA, 23 bp, synthetic construct
  source 1..23: mol_type = other DNA
gagggtttgagctcacagcagtt

SEQ ID NO: 12 - DNA, 300 bp, Glycine max
  source 1..300: mol_type = genomic DNA
gcttagtgcatgtataaataggcatagcggcatagacaattttccctcataaaccgatagaaggtgaccaacttatcgatattaatttattatgcctagatcaacggaccctcttgttattggaggtgtaataggagatgttttggagcctttcacaagtagcgtttctatggggatagtctataataattgccctcaagttatcaactgctgtgagctcaaaccctccaaaatcctcaatcgaccaagaattgaaattggtggagatgacctcaggaccttttacaccctggtattatg

SEQ ID NO: 13 - DNA, 259 bp, synthetic construct
  source 1..259: mol_type = other DNA
gcttagtgcatgtataaataggcatagcggcatagacaattttccctcataaaccgatagaaggtgaccaacttatcgatattaatttattatgcctagatcaacggaccctcttgttattggaggtgtaataggagatgttttggagcctttcacaagtagcgtttctatggggatagtctataataattgccctcaatcgaccaagaattgaaattggtggagatgacctcaggaccttttacaccctggtattatg

SEQ ID NO: 14 - DNA, 233 bp, Glycine max
  source 1..233: mol_type = genomic DNA
ctctctcttatgactttgttgttcctttacaggagaccagaacgaaaccccttagatcttaacaatttgcccgatgagtactctagagatggcaaacaagtcctcgaagaccatacctcttcatccggtaaacttcatgatcataccaatatatatatatatgcacgctgaaatatcaaaggaacactttttttttctttctatttaccgaattctatttcacaatcacacac

SEQ ID NO: 15 - DNA, 220 bp, synthetic construct
  source 1..220: mol_type = other DNA
ctctctcttatgactttgttgttcctttacaggagaccagaacgaaaccccttagatcttaacaatttgcccgatgagtactctagagatggcaaacaagtacctcttcatccggtaaacttcatgatcataccaatatatatatatatgcacgctgaaatatcaaaggaacactttttttttctttctatttaccgaattctatttcacaatcacacac

SEQ ID NO: 16 - DNA, 227 bp, synthetic construct
  source 1..227: mol_type = other DNA
ctctctcttatgactttgttgttcctttacaggagaccagaacgaaaccccttagatcttaacaatttgcccgatgagtactctagagatggcaaacaagtcctcgaagcctcttcatccggtaaacttcatgatcataccaatatatatatatatgcacgctgaaatatcaaaggaacactttttttttctttctatttaccgaattctatttcacaatcacacac

SEQ ID NO: 17 - DNA, 23 bp, synthetic construct
  source 1..23: mol_type = other DNA
ccggatgaagaggtatggtcttc

SEQ ID NO: 18 - DNA, 2436 bp, Glycine max
  source 1..2436: mol_type = genomic DNA
gggggcggggggagataagagagagagagagagtggcagaggaactgatagagaactttcaaagctctttatcttctaccatcactcaaccagcctctatattgcagtctcaaactgaaagtctaaatttttttgtaaaaagctttagttttatccctacccccaccccatctgaaagaaagagtgtttgcctcacatcatttttcccctttctgtctctctctctgtcggtaccatgtaggtcttcccccactactacaccttcacacccttcttttcttcctcctctttctccttctctaaaccctttaactttctctctcttatgactttgttgttcctttacaggagaccagaacgaaaccccttagatcttaacaatttgcccgatgagtactctagagatggcaaacaagtcctcgaagaccatacctcttcatccggtaaacttcatgatcataccaatatatatatatatgcacgctgaaatatcaaaggaacactttttttttctttctatttaccgaattctatttcacaatcacacaccaccatgggaagcctttcacttttcacaggaaacaaagttgaacactgcccgtggtttgttacccatccctgtggtatattccttgaccatgaaacagtgttcaccacacttttttttcctcatttttttattctttttgttttgtccttgtatacgtttcttttctttttttgtttcttctccttttcctcttctacttcgttatagtatctcttaaaagttatggaaaaaataaaataagaaaaacataaaagtagttaggtgatagaatagaacttaaaaaaaagaaaagtagttgtttgggggcgagcggtaagttaattaattgaattgatttgatggaggatttgggtaatcgagaatggggagttaaaggcaaagaagggtgtcatggaagttgctttaattggatggttatagttgcaggttgcaggaaaaagaaaagcggcgggaaggatggaaaagacgagtgtgggaaggtctacgagtgtagattttgttccctcaagttctgcaagtctcaggctcttgggggacacatgaaccgccaccgccaaggctagtaccacctatattattatactttatattttcttcctcttttcttttaccactccttctcacaagaaccaaatctcttctggccgttaatgatttggccatgaaagcttgtcatttcaatcacaaaatgttgtgttgttggattgctgaaattcgatcatcgagagggccccttcagctgttttttttttttctttttgctttgctttattatttgtttttgtgattgttttgagcagagagggaaacggagacgctgaaccaggctcgtcaactggtctttcgttgtgatcataacattgctgcacaaggtgcccctcacttagggtatgcacccattacttccatgcagcgattctcttcctatttcttcttcttgttcaacatttatatatttcatttctcaaatacttgtttttctgtgggttctttagcgtttgttaacgttgttttttcaaggtataacacataatatttgggtactcagcatgacactgtttgatactgcgatttatttgtgataatattcaattcagatgctgccaaacaataggaacggggggttatcatccctcaggagacccaacagtgcctctaagattcccaagatacttctcaggttcatcctcaactcacatgccaccatccccgccaccgccgccgccaccgcaacgaccatacctatacccttcacctacgaggccagtgtcatttgggtcatcacacttccctctccagcatgcagtgaacgattactatgtgggccacgtgatgagtggtggcagccacggacactatgttggaggagagagcacaaggagttacacgtgcattggtgcaccggtggggcaaggtggcggattcgctggtggtaaggaggggtctgcagtgcaggaggaagggttgagtacttggggaaggggctattcaggtgcacaggatcgtttggatcctccctcagcgatcaatcggtttcaagatggtttctaaagagatgagagattctttgtttgagtggtttttggtttgtgttatgtttctgttatgttatgtggtatcatcataaagacacgcaatccagagagagagagaggtggtttttggtttatctacagtaaccagggacagcttttgagctcatggacacgctcagctactttggcttggagttgtgggtgggatttcttctcaacatcgccttttgtattggtagctagctagctagcttggacaaccagttagatttgaactgaaactttccaaactccttttcctttgcatgccaaattacaaccattttccttctcagtaatttgttctttcaa

SEQ ID NO: 19 - DNA, 2423 bp, synthetic construct
  source 1..2423: mol_type = other DNA
gggggcggggggagataagagagagagagagagtggcagaggaactgatagagaactttcaaagctctttatcttctaccatcactcaaccagcctctatattgcagtctcaaactgaaagtctaaatttttttgtaaaaagctttagttttatccctacccccaccccatctgaaagaaagagtgtttgcctcacatcatttttcccctttctgtctctctctctgtcggtaccatgtaggtcttcccccactactacaccttcacacccttcttttcttcctcctctttctccttctctaaaccctttaactttctctctcttatgactttgttgttcctttacaggagaccagaacgaaaccccttagatcttaacaatttgcccgatgagtactctagagatggcaaacaagtacctcttcatccggtaaacttcatgatcataccaatatatatatatatgcacgctgaaatatcaaaggaacactttttttttctttctatttaccgaattctatttcacaatcacacaccaccatgggaagcctttcacttttcacaggaaacaaagttgaacactgcccgtggtttgttacccatccctgtggtatattccttgaccatgaaacagtgttcaccacacttttttttcctcatttttttattctttttgttttgtccttgtatacgtttcttttctttttttgtttcttctccttttcctcttctacttcgttatagtatctcttaaaagttatggaaaaaataaaataagaaaaacataaaagtagttaggtgatagaatagaacttaaaaaaaagaaaagtagttgtttgggggcgagcggtaagttaattaattgaattgatttgatggaggatttgggtaatcgagaatggggagttaaaggcaaagaagggtgtcatggaagttgctttaattggatggttatagttgcaggttgcaggaaaaagaaaagcggcgggaaggatggaaaagacgagtgtgggaaggtctacgagtgtagattttgttccctcaagttctgcaagtctcaggctcttgggggacacatgaaccgccaccgccaaggctagtaccacctatattattatactttatattttcttcctcttttcttttaccactccttctcacaagaaccaaatctcttctggccgttaatgatttggccatgaaagcttgtcatttcaatcacaaaatgttgtgttgttggattgctgaaattcgatcatcgagagggccccttcagctgttttttttttttctttttgctttgctttattatttgtttttgtgattgttttgagcagagagggaaacggagacgctgaaccaggctcgtcaactggtctttcgttgtgatcataacattgctgcacaaggtgcccctcacttagggtatgcacccattacttccatgcagcgattctcttcctatttcttcttcttgttcaacatttatatatttcatttctcaaatacttgtttttctgtgggttctttagcgtttgttaacgttgttttttcaaggtataacacataatatttgggtactcagcatgacactgtttgatactgcgatttatttgtgataatattcaattcagatgctgccaaacaataggaacggggggttatcatccctcaggagacccaacagtgcctctaagattcccaagatacttctcaggttcatcctcaactcacatgccaccatccccgccaccgccgccgccaccgcaacgaccatacctatacccttcacctacgaggccagtgtcatttgggtcatcacacttccctctccagcatgcagtgaacgattactatgtgggccacgtgatgagtggtggcagccacggacactatgttggaggagagagcacaaggagttacacgtgcattggtgcaccggtggggcaaggtggcggattcgctggtggtaaggaggggtctgcagtgcaggaggaagggttgagtacttggggaaggggctattcaggtgcacaggatcgtttggatcctccctcagcgatcaatcggtttcaagatggtttctaaagagatgagagattctttgtttgagtggtttttggtttgtgttatgtttctgttatgttatgtggtatcatcataaagacacgcaatccagagagagagagaggtggtttttggtttatctacagtaaccagggacagcttttgagctcatggacacgctcagctactttggcttggagttgtgggtgggatttcttctcaacatcgccttttgtattggtagctagctagctagcttggacaaccagttagatttgaactgaaactttccaaactccttttcctttgcatgccaaattacaaccattttccttctcagtaatttgttctttcaa

SEQ ID NO: 20 - DNA, 2345 bp, synthetic construct
  source 1..2345: mol_type = other DNA
tcaaccagcctctatattgcagtctcaaactgaaagtctaaatttttttgtaaaaagctttagttttatccctacccccaccccatctgaaagaaagagtgtttgcctcacatcatttttcccctttctgtctctctctctgtcggtaccatgtaggtcttcccccactactacaccttcacacccttcttttcttcctcctctttctccttctctaaaccctttaactttctctctcttatgactttgttgttcctttacaggagaccagaacgaaaccccttagatcttaacaatttgcccgatgagtactctagagatggcaaacaagtcctcgaagcctcttcatccggtaaacttcatgatcataccaatatatatatatatgcacgctgaaatatcaaaggaacactttttttttctttctatttaccgaattctatttcacaatcacacaccaccatgggaagcctttcacttttcacaggaaacaaagttgaacactgcccgtggtttgttacccatccctgtggtatattccttgaccatgaaacagtgttcaccacacttttttttcctcatttttttattctttttgttttgtccttgtatacgtttcttttctttttttgtttcttctccttttcctcttctacttcgttatagtatctcttaaaagttatggaaaaaataaaataagaaaaacataaaagtagttaggtgatagaatagaacttaaaaaaaagaaaagtagttgtttgggggcgagcggtaagttaattaattgaattgatttgatggaggatttgggtaatcgagaatggggagttaaaggcaaagaagggtgtcatggaagttgctttaattggatggttatagttgcaggttgcaggaaaaagaaaagcggcgggaaggatggaaaagacgagtgtgggaaggtctacgagtgtagattttgttccctcaagttctgcaagtctcaggctcttgggggacacatgaaccgccaccgccaaggctagtaccacctatattattatactttatattttcttcctcttttcttttaccactccttctcacaagaaccaaatctcttctggccgttaatgatttggccatgaaagcttgtcatttcaatcacaaaatgttgtgttgttggattgctgaaattcgatcatcgagagggccccttcagctgttttttttttttctttttgctttgctttattatttgtttttgtgattgttttgagcagagagggaaacggagacgctgaaccaggctcgtcaactggtctttcgttgtgatcataacattgctgcacaaggtgcccctcacttagggtatgcacccattacttccatgcagcgattctcttcctatttcttcttcttgttcaacatttatatatttcatttctcaaatacttgtttttctgtgggttctttagcgtttgttaacgttgttttttcaaggtataacacataatatttgggtactcagcatgacactgtttgatactgcgatttatttgtgataatattcaattcagatgctgccaaacaataggaacggggggttatcatccctcaggagacccaacagtgcctctaagattcccaagatacttctcaggttcatcctcaactcacatgccaccatccccgccaccgccgccgccaccgcaacgaccatacctatacccttcacctacgaggccagtgtcatttgggtcatcacacttccctctccagcatgcagtgaacgattactatgtgggccacgtgatgagtggtggcagccacggacactatgttggaggagagagcacaaggagttacacgtgcattggtgcaccggtggggcaaggtggcggattcgctggtggtaaggaggggtctgcagtgcaggaggaagggttgagtacttggggaaggggctattcaggtgcacaggatcgtttggatcctccctcagcgatcaatcggtttcaagatggtttctaaagagatgagagattctttgtttgagtggtttttggtttgtgttatgtttctgttatgttatgtggtatcatcataaagacacgcaatccagagagagagagaggtggtttttggtttatctacagtaaccagggacagcttttgagctcatggacacgctcagctactttggcttggagttgtgggtgggatttcttctcaacatcgccttttgtattggtagctagctagctagcttggacaaccagttagatttgaactgaaactttccaaactccttttcctttgcatgccaaattacaaccattttccttctcagtaatttgttctttcaa

SEQ ID NO: 21 - DNA, 5696 bp, synthetic construct
  source 1..5696: mol_type = other DNA
atcctgcaactgtctgagagattttttttccctcacagcgttttctctccattttaacaattatagaaataaatctaagaagtaaagtaccggaatctactagatgttgaaatattaatatgagtaaacaaacgctagcttagtgcatgtataaataggcatagcggcatagacaattttccctcataaaccgatagaaggtgaccaacttatcgatattaatttattatgcctagatcaacggaccctcttgttattggaggtgtaataggagatgttttggagcctttcacaagtagcgtttctatggggatagtctataataattgccctcaatcgaccaagaattgaaattggtggagatgacctcaggaccttttacaccctggtattatgtaattatgtgttaattgcctttaacttatattatatcttcattttaattgatttattatatatcgaattatctacatacgtacgtttctgtcactctttagggtcgttaataattatggttgattttattgcatgcatggcacttacataatttgcaggttatggtggatcctgatgcacctagcccaggcaatccaacccagagagaatatttgcactggtaattaattaagtatccctccctcgcttccatgatctgctccaccttgatgaaataagtcatcgaaactatacatatatattcacgtacggatgcacgtacgtatttaattacgagttcacttatcttcgatgaattgacttgtgtataaattctctttttaatagactttataaactttaagtaaattcgttcgattcttgagtttattattaactcagtaagttaaaaaaattcaaccaaaatataagtcattctgaattattttctgtctacattcatttagtatgttattttctgataaaataattatcacctttaataatattaaacacatgttctctaataacgttaaactttggatggaaataatctaccacactattataatatctgcacgttattatctagtaatgatatattattagacttggttatttaagtattttataatttataattttttttattattatattgttgaaatatttaattgatatattatttataaatattttattattatttttaaatgaaatagattcttacgaatttataaattggatacaggaggtaagtctataaaattctaaaaaatttacataatctcttcagtttgataatcttttatatatataaaaacatatgatatatggccttgcagtttttagatatattgaagtttatggcaatagggtttataaaaattaatctagatgccttttttatccaccatcatttctctccaaatcaatttaaacttcctcttagagtggtgttaatgtttacgtgagattgcatatatcttgatagatagtctatacttctagcatctacttttcattgactccaaatatatattctgaacttttctcataccttttctctattctatcttttggtaaacatatactctatcatacttagatgctttttaaactttgcatccatatctttttactttctgaaaaatactctgatttttttcaataacaggtataaaaagtacttgtttaaatatttttttttcttccttttcttttttatttggcccctatatgtgaaaaaaaagtttgtaaacgaatctctttttttatttttactcagcaaaaatatgtaactatatttcaggggcacaatgggtgcccaaaccatttacaagaaaggaaataatcctaacatggtagctaaaactcccacggttacacaatattaacatgtaaacgagtctcttgattaagaaaatgtttttacgtagtgtttcctattataatctcatcttataatgctattagatcaagtctgacctgactctaatagatttttaacgtagcttctaacacgtgagaaagctttatacatgttttcattatgcatgatttctatacgtataacacactttgtttcatttaggttcatatatatatatatatatatatatatatatatatatatatatatattgttcaaaaataaaagttctataaaaaaatgtattatcgtcttaactcggatattatttttacaaaagcagcgcccgcttgcgtaagtacttattatgtttagttggtatcagttttgtcaacgctgaaagtacaacaatgaatagcacggagcaaactagttgatatatttcaagagaatcttttgttcaagagaatctttagttatagattattaacatgttcataaaaaaaagaaaagtgaatatcttaataataataaaacattggttaacatatacgtattcatgttttggtgtaaatgtgttaataaactacatgatggatatattttaagataatatttaatatttataatttgttatgcactcgtactaaaaaataaaaatgtgtatattaacaaactcacatatatatatatatatatatataaaagtttgtcaatatacatatatatattttttagtataaatgcgttaataaattatattattgatatattttaagatatattttaattataatttattaatacattcatataaaaaaaataacagtgtttatgttaaaaattcataaataattattaaataaactcacatatatatatatatatatatatatatatatatatatatatatatatatatatatatatatatatatgatatgttatttaatcttctcacctcttataataaaatattcttctcacttgatatatatatatatgacgggtcaagttgaattataaattcaaaaataatatccataattacttattaattattggatttgaacatattgaattaaatttaatccaaatcctcaaaatccaaacccatattaattgggttgatttaaatttataaatgatccatataagtaaacacatttttacttgtgaatcacttttttcttctttctttctatcttttcaaggatattggttgttcaagaataattttacttatgggataataagaaaatagatgttgtggaatgttcaaataaataactatttaggtattattatttctcataaaatatctcatccacgcggatcaacttttaattttctaaatttaaactactaacatgaaataatgttatcttaaagaaaatataaaatattagagaaaattaccataatacctcctgagctttttattaaattacatcttacacataattttatttatttatttattattttacactgattttctttaaatttgaaaatattgtactaacattcggttatatgtttgaaaacattatattgatattttgaaaacattataatgatattctttaatatttctacaaattttatactaacacaccttaccaaagagatgaatataataattgaaaaagtaagtatatttgtgtaattacacgaaatctcagaaattattgttataatttgctcaaaattttatattggacgccactttgcttaaatatattttattaacaaattacgaattgaaattctttttcattgatgaatgaataggttgataaccaatattcctgcaactacaggggcaaacttcggtatgtgcacaattcttattttctatatatcttccttgtacaccttttatatttaattggcagtaaattataaagccttcgtgttattatgtctgatcaaagtaaatctcaaatttctttaaattgatattactttcaatcagttataaaacatcttattaattgtatcagtgttttaaatttagaatataaattaaaaatttatgaagtatatttcaaattataataaaatatattgtttgtttaggtagaaatgtagaatgtaaaaatatatattaaatagaggaaatatttaaaataataaaaactatacgaataaataattcaacaaaatatattgaatttaaaatttaaaataagagagctaaaagttttaatggagagtataaaaaaaagaaagtgaactttattttttcatataagggtctattttaaatttatataaaagtttcgaaatgataacaaaaagttcccaatttataatttgtgattctattttatgaattatattttaagatatcataaaaaaataaaaagtatttgaataagaaactctttaaaaaataaatttaattagtgaaatatttaattagttgcaattatttaaatctttaatttatttttcaaattataaatcagaataatatatcattgaattattgatttacattttataatgattttcttaaaattgtagcataaattatttcaaacattttaacagaatacacaagtattttgagttaatatctatacaaaaaaattattataataattaagaaatttaaatttcatgagcatgattcaaatgacccaaatttttaatccaatttttaattatataagatattcattttcttagaaatgatttacttgtgtgcatatatatcctcgactattcatatcatcaagtgattagttcaccttgctacaggaatgtttaatttgaagaaaaaaaactcaaattcgaattgtttgtatggtaaaaccttttgttgagagagttaatttcgtatgtgtgtttctttctcaaacataataggttatagtggaggatatgtgtgaatgtgatttagataaaagaaaatatgtccatcattattttaattataaaccatattaaatctcgtaagaaatttattaattcgaatgagtatatgataattatttatattttaagatataaattgcatgtgagaaatagtaaatattttgattaagctataaaatttgtgtcatgctgtaatagactgtctttgaactgttatcaatatatgtggatttatttattacttcaataaagggtaatttacaggagaagagattgtctcctatgaaagtccacgtccaatagtagggattcatcgaatagtttttgtgttatttcgtcagctgcgtagactaactctgcaacctccaggctggcgccagaatttcaacactagagactttgctgagatttataatcttggattaccagtagcggccatgtacttcaactgtaaacgagaaaatgatcaaagcagtggaagaagaagataatagaaagaaaactattattaatatatatcccgagtttttatcagctcaacatgaaataataaataacttctttttatatttaagtatttattaaatataaatatatattatataaaaaaatcaacacgaatttcaattaaaaaataatattagtatgaaagagctaatggcccctaaagaaaaaggtgcctagggcatcagcaataagcttagtatgaaggtcatttggacatacctgccaaactgaagatcttcagacgagccattaatttcccagcaaggtaatctgcaatctgattagcttctctgaatgtgtgttggaacaccagattccactcaagctgcatgaaggttctgatcttggtaatcaaaggagacacattcacaaataattatgtacgtatgaggaaataatcgattaatctacttgttcttcaaactcatatttatgaagagcaaagtaattgattaattctgttgattttataaataacatctttgttttttcattg

SEQ ID NO: 22 - DNA, 2819 bp, Glycine max
  source 1..2819: mol_type = genomic DNA
ggggagggaggacccacacacacaggattacacatgatgctttttttgacctgatcgagctgatgggagagaaaaaactgggaaattttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctctagctaaatctaaattatgtattgcaacatattttaaaggaggggcaaaacacactcgatctaccttcaaagaaacttttaagacattataaatatggagcccacactcccttcttcctcagtaagatcatcaacttgtattcctcacttccttagctcctcctcttccttgttcctcctcttacaatggcaaaaatgcctttagagcctctaatagtggggagagtcataggagaagttcttgattctttcaccacaagcacaaaaatgactgtgagttacaacaagaagcaagtttacaatggccatgagctcttcccttccactgtcaacaccaagcccaaggttgagattgagggtggtgatatgaggtccttctttacactggtatatatatttcttttatctcttctcttcttattccttttcttctttaaagaacaagatttttttggggaaaaaaaaagagaaaaaacttaagctgttgccagtgtgtttctttgttattttctgtaatcatggtcacacggtatgtctcttttattggagttttctttacaaatactgaatcattaagcaaatgtccccctttttcggtgcagatcatgactgaccctgatgttcctggccctagtgacccttatctgagagagcacttgcactggtacttaatataaaaattaactaacttaagtagtcatttattacataaataacacaccacacccaccccacatatattgttataccggtttctcttaataacttaacctctttaaaagttatgtactggttatccatctgatattcacgttagatgtctgtgtattttattatcaagtttgaagttttgcaagatatatgcattcctttcatcacatcaaactatggacagccaggaaggtaataacaaatacttacatcacatgaagaaggtacacacatgcctttgtattctgcaaaagtagcttgttccaccataatcccatccaccctaaaatcaaaacaatctaaaagggtgaataaatctcgaagtctcaaacaaacattacagaactacatataaggcaaacaaccagtaaccattcattcattcataggtatctaacagtcaataattttgttgcaaacattctttttgctgcaggatagtgacagatattccaggcacaacagatgccacatttggtaggttcatatgaaaaatgtgtatatatgggaacttactttgatgatcatgagagagatcactgacaaaaaatgatattcctaataatatgtagggaaagagttggtgagctatgaggtcccaaagcctaatattggaatccataggtttgtgtttgtcctgttcaagcaaaagcgtagacagtgtgttactccacccacttcaagggaccacttcaacacacgcaaattcgcagcagagaacgaccttggcctccctgtggctgctgtctacttcaatgcacagagggaaacggctgcaagaagacgctagctatagctaagtagctgctggttttgccactgcaacaaccaaagtagtattgtattgaataaagcgataaaaaggtacaagtacaaggagtttcagtagtggaagttgatcttcacatgtggcttcaaataacttgcagaaagaaggaagataatcattttctagtttgactagtgtgtatgctatgttttactttccatcagatgttgtgtacgttctgtagtataagtgtactacgtgtattattattacctcgtgggtactactatgaggtgatcttatatatatatatataaagaaataagaggtttggtaggccatgtcgtttcttttcctttaatagtttagggtttggcttcgtttcttctttccttgcggtttcgatcactcagtaccttctctctattggtttttttcgatctcttcacaactaaattacgtttttgacagaaaaaatggtattattaattaattactatatgagactttatgatcagtgacttgtacacactttcgatcttgtcatgttgattatttttcctcaagatgcgaaacatttcactttcagtaatacgaggcttgctcgtctatataatatttatgacatctgctttttgctaacttgatggattgacccttcaggaaacagtgatttctacaccaaatttccagcatatattcacactgctaaaataacatgtgattagtgtgtatggaaactgttaatatgcactcctttaaatgcaggtgtaatcgttccaatgccctaaacatagaagctatgaaataaaatggcgttatgttgaaatcaacacatt

SEQ ID NO: 23 - DNA, 522 bp, Glycine max
  source 1..522: mol_type = genomic DNA
atggcaaaaatgcctttagagcctctaatagtggggagagtcataggagaagttcttgattctttcaccacaagcacaaaaatgactgtgagttacaacaagaagcaagtttacaatggccatgagctcttcccttccactgtcaacaccaagcccaaggttgagattgagggtggtgatatgaggtccttctttacactgatcatgactgaccctgatgttcctggccctagtgacccttatctgagagagcacttgcactggatagtgacagatattccaggcacaacagatgccacatttgggaaagagttggtgagctatgaggtcccaaagcctaatattggaatccataggtttgtgtttgtcctgttcaagcaaaagcgtagacagtgtgttactccacccacttcaagggaccacttcaacacacgcaaattcgcagcagagaacgaccttggcctccctgtggctgctgtctacttcaatgcacagagggaaacggctgcaagaagacgctag

SEQ ID NO: 24 - protein, 173 aa, Glycine max
MAKMPLEPLIVGRVIGEVLDSFTTSTKMTVSYNKKQVYNGHELFPSTVNTKPKVEIEGGDMRSFFTLIMTDPDVPGPSDPYLREHLHWIVTDIPGTTDATFGKELVSYEVPKPNIGIHRFVFVLFKQKRRQCVTPPTSRDHFNTRKFAAENDLGLPVAAVYFNAQRETAARRR

SEQ ID NO: 25 - DNA, 6448 bp, Glycine max
  source 1..6448: mol_type = genomic DNA
agtaattgaaatgatgagagtcaatgcggggagtgaccttgtgagggccatgcgtgtggggggagtggaagcccataccgttgcaaagagcaatggtgaagtgaatcaaagtagcaaaaagcaaaagcaaggtttgagcggagcggaacgcaacgcaatgcaagtggacaaagccaatttattcacccaagaaaccccccaaccccaacaaaataaaataaatacactaaataaaccttaaccgataatattagtataatgatgtaaaattctatagcatttatataacaggagaatttatttgtatttggagagaagaagaaataaagcttatagttatagtgagaagagaaggaagattagttgctaataattaagacagaaacatacgtggcagcttagggttcaccccaagtcctaaaaacgccccctccctacccttcccccatttcccccccaacccaaacgctgccaacaacaacactctctctacaaaattgatggtcccaccgacgagcgggtgtcggaatcggaggccatgaacggcggcgccaccaccgccaccttccgatccatcctcgacaagcccctcaaccagcttaccgaggatgacatttctcagctcactcgcgaagactgtcgcagattcctcaaagaaaaaggtcccttctctttctctctcttctctcctccatgatttccggcacctgaaaaacctccattcccctaaatacattatcatattatttattaaaacattaatcgctcgctcgggatgcgggttttttaaaaattaaatttaacgagaatgatattaatagttaattaattaaaatgtcgaagacacgtgttcgtacagggatgcgtaggccttcctggaacaaatcgcaggcgatccaacaggtcatttccctgaaagcgctgctggaaccttccgacgatgatactcctcctcctcctcctcccgccatgcaccaccgtagccatgctcaacctcaacctcaagtgagttacttactactttcttctttctttcgcctttgcttctggaacctcaaatatcctcgtgtgcttgtaggtgaatttgagtgaacctcctcctcctccgcccaaggctccgccacctgaagaacccgcttttcatgctgctgaagacattcagaaatctgcgtcgtctggggaaaagcctacggaaactaatgacaccaacaccaacgttgctagccccaagtttgtttttctctctctctctctctctcttgatggtttttaatgcctttttgtgtgaccggtatagttattttttaataatttttttaaggttttagggtgtggaatctcgtccaagctagttggatctttgatgttgctttcggtactttgtgggaagaaaatagtgtggttggtaaagtgtgctttggagttcagtgaagagtgtaggttggttaaaacttgaaacgttaaggtttgcttttttaagtggattttcttttggcattagcttggaaaattggctcattgtgttgtgttatttattattatttttttctttttcgtgaggtggaaaatgatacggacggtgggtttttggtatccgttgaatgtgtatgggtggggaaagtggaggaaaagctgttagtgaactagtcttagctcttaggtatgtttggatacatttctccataaacactttaggtgaaaaaaaatagaaaataaaatgagttggacttctactataagctaatgttaaattttatataagctttcacttttaacaatttaacttatcctaaaactgtagtgtatataggttggttttagtttatgggagaagctcaattcattttatcttcttattttcgcctatcatcagtacctataaagaagtttatcctaatagggccttgaaacagtttgtatattcctgcagtttaagaaaatgttaggtgtatttctgggatcatgggttggatctttctgactgtgaggttaggaaaaggtttatggccattaatttggatgtttggatttttctgggatgtattaggtgggttctttgttttttttagcagggttggttttattttttgggtcggggtggggagaagaatgattggaaagtgctaccttctagaagcactaaggatttctgtccaattccaaacaaccagtcagagcatgcgaagcaaatatctgttatttccaaatctagatatccccataactttaatattgttgttattctatatttgttgcattgtgtttgcactttgttgactaattaatatttaattgttgttttttgttttccattcatgtttgaccttatacgattgtgtcatactactttaatttaaaattttctctttagatatcatgattgaggtggagaaaggttcatgcggattaaatacgtggcaaatatcaaaaggaggaaggggaattctttaggtgtagaagtgattcttttaggttttaacatcatgaatttcaagattcaaggagtacaaataaataatagtgaagtttccatccataagtttgtgttggtatctatgtcaagtcttgtttccttattagtagcatccaaataaatgaacctgtttatttgtggataaccaataacccttgatttcagccatggatatgctaggaagtcatatgtcttgttgatatggaatacctgggagattttgctttcagctgcagcaagtggtaatcatacctagctgaaaagaaagctgacatgagtattataattaggtattagggacctggatgaaaaaatagagaaaagaagagaaagatttaatttcttgattaaaatgaacagaacaaaggaaaactaaatctcctccaagggtttccccttgacgaagggtttctgttttcaaaatagaactaatgctcagttttccaaatgataacaaaaacgtaaggaagcctcttcccttgtatccttttttttttatcttattatttactaaccccctctatctatctaactaactaactaactaatattccaactgtccctaactaacttccccttcccctctgccttaacgttaagtgttatagatttactcaagtgtcttcccagattcatgtttaaattaaaatcggctcttgtgcagttctttacaaaaataggagtccaggatagtaagtgtacaaaactttcattatatagttactttccgtatgaatggagcgatttggtaacctaaaatgtcagatagtttaaattcaatacatttgcataaaatatacatgactggtggcaggccagtgttccttttatgtagtattgtccattcatactcttaacattacttacaaatgtggtcatagagggtgtgcaactagtggatcatttgggcaaatgacaattttctattgtggtaaggtgaatgtctatgacagagtctcgcctgataaggttggtaatgatctatcctcttgcatcaatagctggccataatccaataagacttcaccttccaatgcaaccttatttggatttgcaggcacgagcaatcatgcagcttgcaacgagtccagtccagcttactcaggatgatcctttaaatggaaatgcagctgtttggacttctccttgccacttaccgatggataaggatgtcctcgttcctgttgatactacaatccttcaggttgctcaagcaggtagcaagtgggttcaaagattgcttgattttgctctgaagtagtagtatgaagtactttgatgtgcaaggaaatgagtcttcttcagtgcccattgcatcaaaatctggaattcttcaaggaatgatctcatctgatgagcagatgaagaaaatgattgtagcctttttattagcttaactgccggattcaatttatttcttaaaagaggatgcagtgctaagttgccttgctatgtcttacagaaaaaaatcatgaaatagggttcaagctaattgttgctttttattttactatttcatgtgccatgttcgttgagtttttcatgctattggttgttcctttgctgtgcatgttgaaaagtattttgatgtccagtcattaaaatgaccacattccctctgaatgctgattttttgaattatagcctttgagtctgtgtgtcttgaagatggacaagcataacatctttgacttgcttaaatcatgataacttgcataatttgaaattaaagatgttaagatccacatatttacttttgtgatattgctaacttcataattggtgttccgaattgttttttacagataagatggtggagtatcctctgcaatatagggagaaagggagcatagctcgtgatgctggtaagtattattaactgtttggaattttcagtctgctagaacttgttctattgtgaggaaaatttacagaaaatctcgaagtagaaaaataaatttctgataaactagtgagctgaatttggacccatgctacttcccttattgaatctctttcaacatgtcctacccatttcttttcactgatttggcaagtctttggagacccttttttgttttttttcctaggcataccaggtcattattttttttaatcaagaaaaacaaaattcgccaacttgatttaagtctctttcttggataaaacacaatggctttgtttgatcattgtagtccatggaaaaacatgcctggttaacagaacttcctgttctttgtgaatctaaaagcaatgaaatatgataaaatcttgtgggtttgatgtactatatcatcacaactttaatgtcatatttatgcacccacatggcagcataatattcatcttagattgcaagtatctttatgagttacagagaattgtttaaacttcaaagcattgtaataagttttcatacatcaaattcctagaacacaagtattcataatatatgtatatatctgtttcataagatgtagagggtcaggaacaccgaaaagtgtcattgcaacgatatcttgaaaagcgtaaggacaggtatttgcagtactaaactgccatttgtttatagacatcaatttcatccattttgttacaagtgtgtagtttagtgatagtctgctgaattattctcttaatctgccctgtatgttaggggaagattgaaaggcaagaaattgactgggataacttcatctaacttcgagatgtatttgaaccttccagtgaaggtccattcctcaaatgggaattcaagccgtagcagtactagctctccaccacaacctagactgcctctagtttccagtggttcagatcagctaaaggttgcccttcccattgatctcaatgacaaaggttagttgcaatctttcttcaacgttaagtaacaaaaccttttttttccttacaagaatgtgataattagtcttaaagcttttaagattatatcagagaaatataataattgaattgaatctgattgtcctgcttgctgtcttgatatttggggaagtgagatttgcaaaccatctgacaagcagtgggggttctttgtgtgcaatgcttccaagacaaccttataaaccggtgttaatcttttacgactagtttacaaagtataatatttgtggtggtgattaaaaattaaatggtgaaccaagatatgataaaccgtcagcctcgatactaggttaccatttgttaaacaatattttatgtcatagatttatgttgtctcagtgtctttgcagatgttcaagaatgctaaaattcaaactagatagattatgcagagttgggactcgtagatgattgaagagagatgacgaaagtattataactgaagagaccatagcgtctagcagcctactgcaagaattggattgctcttagactggtcaattttagtatatttaactgtttgggcaaatttcttttgtttaggatagtggagtgtcctgcctacctgatcaattttgtaatctaatttcatccttcgggaatatcttgttactgaagttttaaggttttatgagttgaaatgtagagccaggagcagctattttcccaggaatatatgatatgtaaattcatcgtgctgcctcttgttaaatgttaatagcttttgtgcaagccaaactattctcttactcttcttatatcttatgtttgttcactctagccaaactattctcttactcttcttatatcttatgtttgtttactctattggtgctgatttcgattttgtttcatgacaaacaactttagtaatttcactgaacgttgatattttgtaggataaatctgtcacgctatactgccattttttttttttggccacagacagttttttttaatcttttatattgatgtgaagtatttattaattttgtaatgactggaaaatttgagtttaatttatatcttaatttaaaatattatatattttttgtgacaggaaaaaacttg

SEQ ID NO: 26 - DNA, 7320 bp, Glycine max
  source 1..7320: mol_type = genomic DNA
agtgtcacgtactacatcaaaaaaaaaaaaagtgtcacatacacatataatgcaccacatcgacgaaatgctaacttcaacgaaagagaactaatggttgaactcacatgataaacaaaatatagtttcagagactattttatgaatttctttcaatccaaaaagtaacgttacaacgaataacagattcaagaattaaagcgagtaattactcttttatcattacttgcaatgacctaattgtagataggactaaagactcctaatttgaaaatatttacctgttatgttgttactcttgattggggtataatttcttattgtaaacatatttaatctaccttgcaattgggttatacattgtggatttcaacttctcatgagtctttaggttgacattgtagttaggtcgaactagatcgatctatccagaatgaattcgattatttggaggcaaaacaaaattaattttgatcaaatgaatattaattaaattattaaatataggaaaactatgtattttatgaagacgtcattttttagaaagtagtctttttaaccttaagaaaggtaatagtaattgagatgatgggagtcaatgcggggagagaccttgtgagggccatgcgtgtggggagtggaagcccataccgttgcaaagtgcaatggtgaagggaagaaaagtagcaacgcaagtggacaaaggcaatttattcacccaagaaacccccaaccccaacaacacacaaataattacaaagagacgccaacttccacgcagaaataaaagtaaataaatgaacagtaagtaaagcttaagcaatgatgttgacccaattcgaggtatttatatttatgggaataatttatttgatgaaaaaatttatatatatatatatatatattttttttttacgtaaaattcatttggatagcattaatctacgatgaaatagattaagatatctgaaatctcaaatataaaaaaaaataaaaacgtatagttataaggataagagaaggaagattagttgtgaataattaagacagaaacatacgtggcagcttagggttcaccccaagtcctaaaaaagccctcccagccttcccccatttccccccaacccaaacgctgaacaacaaccacactctctacaaaattgatggtcccaccgacgagcgggtgccggaatcggaggccatgaacggcggtgccaccaccgccaccttccgatccatcctcgacaagcccctcaaccagctcaccgaggatgacatttctcagctcactcgcgaagactgtcgcagattcctcaaagaaaaaggtctctctctctctctttctctctcttctctcctccatgatttccggcacctgataaacctccattcccctagatacattattatattataattaaaacattaatcccttgctcgctcgggatgcgggtttttttaattataaattaaatttaacgagaacgatattaatgaattaataattagtaattaaaatgtccaagacacgtgttcgtacagggatgcgcaggccttcctggaacaaatcgcaggcgatccaacaggtcatttccctgaaagcgctgctggaaccttccgacgatgatactcctcctcctaccgccatgcaccaccgtagtcatgctcctccccctccacctcaacctcaatctcaagtgagttactttcttcttcctttcacctttgcttctggagcctctaatattatctcatgaatcgtggttgtaggtgaatttgactgaacctcctcctccgcccaaggctccgccacctgaagaatcctcttttcatgctgctgaagacattcagaaacctgcgtcgtctggggaaaaaccttcggaaactaatgacaccaacaccaacgttgctagccccaagtttgtttctttctctctctctctctctctctctctctctctctctctccatgatttttaatgcctttttttgtgtgaccgtcatagttctttttttaatcattttttttaaggttttggggtgtggaatctcgtccaagctagttggatctttgatgttgctttcgaaactttgtgggaagaaaataatgtggttggtaaggtgtgttttggagttcagtgaagagtgaaggttgtggactagagggactagataggaggatttgggtggttaaaactttaaatgttacggtttgcttttggtgttaggttggaaaattggctcattgttttgtttcttaaaattttttgtgaggtgggagatgatacggacggtgggtttttggtatccattaattgtatatgggtgggctaagtggaggaaaagctgttggtgaactagtcttaagggtctgtttggataaatttttccataaactcttcaggagaaaaaaaagaaagaattggacttctcctataagctaatgttaaattttatagaagctttttcttttaacaatttaacttctccaaaaactgaagtgtataagttgtttttagcttatgggagaaactcaattcgttttatattcttattttttcctatacagaagtttgtcgtgggatcatgggttggtgcttaaagtgaggttagaaaaaggtttatggtcattaatttggatgtgtggatttttctgggatgtattaggtgggtttttttaggaggggggggggggggggatgattggaaagtgctacctccagaagcactatggatttctatcaattccaaacaaccagttggcgcatgggaagcaaatatctgctgtttccaaatctagatatccctgtaactttaatattgttgttattctatatttgttgcattgtgtttgcacttttgttgactaattaatatttaattgttggtttttgttttccattcatgcccaaccttatacgattgtgtcatgctttaatttaaaattttctatttagatatcatgattgaggtggagacaaggttcatgcaggttaaatatcaaaaggaaggagggaagttccttaggtgtaaaagtgattattttaggtttaatatcatgagtctcaagattctaggagtacaaacaaataagagtgaagtttccatccataaatttgtgttggtctctgtcaaggcttgttttgttatttctagcatctaaacaaatgaacctgtttatttgtggataaccaacaacccttgatttcagccatggatatggtaggaaattgtatgttatgtggatatggaatctcttggaaattttgctttcagttgcagcaagtggtaatcatacctagctgaaaagaaaactgacatgagtattataattaggtgttaggaacctggatgagaaaatagagaaaagaagagaaagattgtatttcttgattaaaaatgaacaaggcaaaggaaagctaaatctcctccaagggtttccccttgacgaagggtttccatttccaatagagctaatgctcagttttccagatgataacaaaacagaatgaagccacttcccttgcatccttctatttatttaccttactaacctcatctaactaactaatattcatgtttaaattaaaaactgcccttgtgccattctttacaaaaataggagtctaggatagtgattaactgttcaaaacattctttacatagttactttccctatgaatagagcgatctggtaacctaaaatgtcaattagcttagattcaatacatttggcattaaaaatacgtgactagttgctggccagtgtttcttttatgtattgtccatttgtactcttaacattacttacaaatgtgatcacagagggtgtgcaactagtggatcatttgggcaaatgacaattttctattgtggtaaggtgaatgtctatgatggagtctcgcctgataaggttggtaatgatccatcctcttgcataaatagttggccataaatccaataagatttcacctttcaatgcaatgtatttggatttgcaggcacgagcaatcatgcagcttgcggtgagtcctgtccagtttactcaagatgatccttcaaatggaaatgcagctgtttggccttctccttgccacttaccaatggataaggatgtcctcattcctgtagatacaacaatccttcaggttgctcaatcaggtagcaagtgggttgaaagattgcttaattttgctgaagtattggtactttgatgtgcaacggaatgagtcttcttcagtgcccattgcataaaaaattggaattcaaggaatgatttcatctgatgaacagatgaaggaaaatattgtagtctttttattagtttaactatcctgattcaatatatttcttaaaggaggatacaatgccaagttgccttgctttgaaatcatgaaatagggttgaagctaatagttgctttttgttgaactatttcatgtgtcatgttcgttgagtttttcatgctactggttgttccgttgctgtgaaagttgaaaaataattggatgtccagtcattattatgatcaaattcactctgaatgcctaatattagaattatagcttttgagtctgtgtcttgaagatggacaggcacaacatctctgacttgataaaatcatgataacctgcataattttgaactaatgttaagatccacatatgtacaccttttgcggtatttataacttcataattggtgttctgaattgtattttacagataagatgatggaatatcctctgcaatatagagagaaaggtagcatagctcgtgatgctggtaagtataattaactatttggaattttcagtctgctagaacttgttctgttttacggcaaacttgcagaaaattttgaagtagaaaattgaaatttctgataaactaatggcctgaatttggacccttgctacttcctttattgaatttcttttaatatgtcctccccatttcttttcatttgatttggttagtctttggaggtccttttttgtttttttcctaggcatactaggtcatggtctttatgaattaagaaaaacaaaattcacctacttgatttaaggttctttcttggataaaccacaatggctttctttgatctatttagcccatgggaaaaggcttttgttgttattgctaccattgtcctagagttatgatgttatgccacaatttttctacctttgttttgaatgtaagctccacttttgagtgcaagagcttcttgtatactttaccatcatgtctgctggtaaaattgctactattagctgtaaaccccctccctccggcatgcacacaaacaaaaaaaaaagctaccattagaattgcagtgtatttctagattcagctgtgttgtgtgcacttctaaattgatcatatatgtatgggtagtggtgctgatatcattcatcaattatgtcccccctactggtgctcataattgccagccattttagtatctaggtaatgtgcacctgctccacttaaatatctggtgaacagaacttgttcttcgcgaatctaaatgcaatggaatatgataaaatcttgtaggttggctgtactttatcattacgggtataagaggatgatttttgttgtaataaattgtcaaaatatcaaataagtgatatacaactttaatgacatatatttatgcacccacatggtagcataatattcatcttagattgcaactatctttaagaggtacagagaattgttgtttaaacttttaaagcattgtaataagtttccatacatcaaattgttacaacttaagtattcataatatatatatatcgctttgataagatgtagagggtcaggcaagcagaaaagtgtcattgcagcgatatcttgaaaagcgtaaggacaggtatttgcagtactgaactgccatttttatagacatgtattccagccattgtgttacaagtgtatatacttttagtgatagcccgctgaattattctcttaatctgccttctatgttagggggagattgaaaggcaagaaattgactgggataacttcatctaacttcgagatgtatttgaaccttccagtgaaggtccatgcctcaaatgggaattcaagccgtagcagtactagctctccaccacagcctagactgcctctagtatctagtggttcagctgacaaccagctaaaggttgcccttcccattgatctcaatgacaaaggttggttgcaatcttctcttcaactgctaagtaacaaaacacattttttcttacaagaatgtgataattagccttcaacaagattttaaaattatattaataattgatgttaccttgtaaactgcttctaagagaaccttgtaaactgatgttaccttttatgactagtttacatataatatttgtggtggtgattaaaatcctaatgttgaatatgatgaacggtcaatctgaatgctaggtaactaaagtatttttcaatattttgtcataaatttatgttgtctcagtgtcattgcagatgttcaagaatgctaaaactctaactagatagattatgcagagttgggactcgtatatgaatgattgaagagagatgatgaaagtattataactgaagagaccatagcgtctagcagcctactgcaacaattggactgctcttagactggtcgattttagtatcttttaactgtttgggcaaatttcttttgtttaggatagtggagtgtcctgcttacctgatcaattttgtaatctaatttcatccttcgggaatttcctgttactgaagttttaaggttttatgagttgaaatgtagagccaggagcagccattttccgaggaatatatgatatgtaaattcatggcgctgcctcttgttaaatgttaatagcttttgtgcaaagccaaaccactctcttactcttctcttacgtttgcttactctcttgttgctggttttgattttctttcagcatacgaatgacttgagtaatttcattgaatgttgatattttgtaggataaatgtgttacgccataatcaataatgccaaccttattttggccgcaaacaacctttgaattgactagaacattctggtttaatttatatcttcatttaaaatattatatgttgtgaccggaaaaatatgattaattatataaatgtctaaatttattgtcatgtgatgtaaaaagtgatattctgaaattttacacatttatctgccacaaaattagacagttaacaagagggtgcaagcactcaaacaaaacgacgataacgatatttttttttttcgggtggaaacacccgc

SEQ ID NO: 27 - protein, 337 aa, Glycine max
MNGGATTATFRSILDKPLNQLTEDDISQLTREDCRRFLKEKGMRRPSWNKSQAIQQVISLKALLEPSDDDTPPPPPPAMHHRSHAQPQPQVNLSEPPPPPPKAPPPEEPAFHAAEDIQKSASSGEKPTETNDTNTNVASPKGCATSGSFGQMTIFYCGKVNVYDRVSPDKARAIMQLATSPVQLTQDDPLNGNAAVWTSPCHLPMDKDVLVPVDTTILQVAQADKMVEYPLQYREKGSIARDADVEGQEHRKVSLQRYLEKRKDRGRLKGKKLTGITSSNFEMYLNLPVKVHSSNGNSSRSSTSSPPQPRLPLVSSGSDQLKVALPIDLNDKDVQEC

SEQ ID NO: 28 - protein, 350 aa, Glycine max
MNGGATTATFRSILDKPLNQLTEDDISQLTREDCRRFLKEKGMRRPSWNKSQAIQQVISLKALLEPSDDDTPPPTAMHHRSHAPPPPPQPQSQVNLTEPPPPPKAPPPEESSFHAAEDIQKPASSGEKPSETNDTNTNVASPKGCATSGSFGQMTIFYCGKVNVYDGVSPDKARAIMQLAVSPVQFTQDDPSNGNAAVWPSPCHLPMDKDVLIPVDTTILQVAQSDKMMEYPLQYREKGSIARDADVEGQASRKVSLQRYLEKRKDRGRLKGKKLTGITSSNFEMYLNLPVKVHASNGNSSRSSTSSPPQPRLPLVSSGSADNQLKVALPIDLNDKVSLQMFKNAKTLTR

SEQ ID NO: 29 - protein, 67 aa, Synthetic construct
MNGGATTATFRSILDKPLNQLTEDDISQLTREDCRRFLKEKGMRRPSWNKSQATGHFPESAAGTFRR

SEQ ID NO: 30 - protein, 58 aa, Synthetic construct
MNGGATTATFRSILDKPLNQLTEDDISQLTREDCRRFLKEKGMRRPSWNKSQAIRSFP

SEQ ID NO: 31 - DNA, 7315 bp, Synthetic construct
  source 1..7315: mol_type = other DNA
agtgtcacgtactacatcaaaaaaaaaaaaagtgtcacatacacatataatgcaccacatcgacgaaatgctaacttcaacgaaagagaactaatggttgaactcacatgataaacaaaatatagtttcagagactattttatgaatttctttcaatccaaaaagtaacgttacaacgaataacagattcaagaattaaagcgagtaattactcttttatcattacttgcaatgacctaattgtagataggactaaagactcctaatttgaaaatatttacctgttatgttgttactcttgattggggtataatttcttattgtaaacatatttaatctaccttgcaattgggttatacattgtggatttcaacttctcatgagtctttaggttgacattgtagttaggtcgaactagatcgatctatccagaatgaattcgattatttggaggcaaaacaaaattaattttgatcaaatgaatattaattaaattattaaatataggaaaactatgtattttatgaagacgtcattttttagaaagtagtctttttaaccttaagaaaggtaatagtaattgagatgatgggagtcaatgcggggagagaccttgtgagggccatgcgtgtggggagtggaagcccataccgttgcaaagtgcaatggtgaagggaagaaaagtagcaacgcaagtggacaaaggcaatttattcacccaagaaacccccaaccccaacaacacacaaataattacaaagagacgccaacttccacgcagaaataaaagtaaataaatgaacagtaagtaaagcttaagcaatgatgttgacccaattcgaggtatttatatttatgggaataatttatttgatgaaaaaatttatatatatatatatatatattttttttttacgtaaaattcatttggatagcattaatctacgatgaaatagattaagatatctgaaatctcaaatataaaaaaaaataaaaacgtatagttataaggataagagaaggaagattagttgtgaataattaagacagaaacatacgtggcagcttagggttcaccccaagtcctaaaaaagccctcccagccttcccccatttccccccaacccaaacgctgaacaacaaccacactctctacaaaattgatggtcccaccgacgagcgggtgccggaatcggaggccatgaacggcggtgccaccaccgccaccttccgatccatcctcgacaagcccctcaaccagctcaccgaggatgacatttctcagctcactcgcgaagactgtcgcagattcctcaaagaaaaaggtctctctctctctctttctctctcttctctcctccatgatttccggcacctgataaacctccattcccctagatacattattatattataattaaaacattaatcccttgctcgctcgggatgcgggtttttttaattataaattaaatttaacgagaacgatattaatgaattaataattagtaattaaaatgtccaagacacgtgttcgtacagggatgcgcaggccttcctggaacaaatcgcaggcgacaggtcatttccctgaaagcgctgctggaaccttccgacgatgatactcctcctcctaccgccatgcaccaccgtagtcatgctcctccccctccacctcaacctcaatctcaagtgagttactttcttcttcctttcacctttgcttctggagcctctaatattatctcatgaatcgtggttgtaggtgaatttgactgaacctcctcctccgcccaaggctccgccacctgaagaatcctcttttcatgctgctgaagacattcagaaacctgcgtcgtctggggaaaaaccttcggaaactaatgacaccaacaccaacgttgctagccccaagtttgtttctttctctctctctctctctctctctctctctctctctctccatgatttttaatgcctttttttgtgtgaccgtcatagttctttttttaatcattttttttaaggttttggggtgtggaatctcgtccaagctagttggatctttgatgttgctttcgaaactttgtgggaagaaaataatgtggttggtaaggtgtgttttggagttcagtgaagagtgaaggttgtggactagagggactagataggaggatttgggtggttaaaactttaaatgttacggtttgcttttggtgttaggttggaaaattggctcattgttttgtttcttaaaattttttgtgaggtgggagatgatacggacggtgggtttttggtatccattaattgtatatgggtgggctaagtggaggaaaagctgttggtgaactagtcttaagggtctgtttggataaatttttccataaactcttcaggagaaaaaaaagaaagaattggacttctcctataagctaatgttaaattttatagaagctttttcttttaacaatttaacttctccaaaaactgaagtgtataagttgtttttagcttatgggagaaactcaattcgttttatattcttattttttcctatacagaagtttgtcgtgggatcatgggttggtgcttaaagtgaggttagaaaaaggtttatggtcattaatttggatgtgtggatttttctgggatgtattaggtgggtttttttaggaggggggggggggggggatgattggaaagtgctacctccagaagcactatggatttctatcaattccaaacaaccagttggcgcatgggaagcaaatatctgctgtttccaaatctagatatccctgtaactttaatattgttgttattctatatttgttgcattgtgtttgcacttttgttgactaattaatatttaattgttggtttttgttttccattcatgcccaaccttatacgattgtgtcatgctttaatttaaaattttctatttagatatcatgattgaggtggagacaaggttcatgcaggttaaatatcaaaaggaaggagggaagttccttaggtgtaaaagtgattattttaggtttaatatcatgagtctcaagattctaggagtacaaacaaataagagtgaagtttccatccataaatttgtgttggtctctgtcaaggcttgttttgttatttctagcatctaaacaaatgaacctgtttatttgtggataaccaacaacccttgatttcagccatggatatggtaggaaattgtatgttatgtggatatggaatctcttggaaattttgctttcagttgcagcaagtggtaatcatacctagctgaaaagaaaactgacatgagtattataattaggtgttaggaacctggatgagaaaatagagaaaagaagagaaagattgtatttcttgattaaaaatgaacaaggcaaaggaaagctaaatctcctccaagggtttccccttgacgaagggtttccatttccaatagagctaatgctcagttttccagatgataacaaaacagaatgaagccacttcccttgcatccttctatttatttaccttactaacctcatctaactaactaatattcatgtttaaattaaaaactgcccttgtgccattctttacaaaaataggagtctaggatagtgattaactgttcaaaacattctttacatagttactttccctatgaatagagcgatctggtaacctaaaatgtcaattagcttagattcaatacatttggcattaaaaatacgtgactagttgctggccagtgtttcttttatgtattgtccatttgtactcttaacattacttacaaatgtgatcacagagggtgtgcaactagtggatcatttgggcaaatgacaattttctattgtggtaaggtgaatgtctatgatggagtctcgcctgataaggttggtaatgatccatcctcttgcataaatagttggccataaatccaataagatttcacctttcaatgcaatgtatttggatttgcaggcacgagcaatcatgcagcttgcggtgagtcctgtccagtttactcaagatgatccttcaaatggaaatgcagctgtttggccttctccttgccacttaccaatggataaggatgtcctcattcctgtagatacaacaatccttcaggttgctcaatcaggtagcaagtgggttgaaagattgcttaattttgctgaagtattggtactttgatgtgcaacggaatgagtcttcttcagtgcccattgcataaaaaattggaattcaaggaatgatttcatctgatgaacagatgaaggaaaatattgtagtctttttattagtttaactatcctgattcaatatatttcttaaaggaggatacaatgccaagttgccttgctttgaaatcatgaaatagggttgaagctaatagttgctttttgttgaactatttcatgtgtcatgttcgttgagtttttcatgctactggttgttccgttgctgtgaaagttgaaaaataattggatgtccagtcattattatgatcaaattcactctgaatgcctaatattagaattatagcttttgagtctgtgtcttgaagatggacaggcacaacatctctgacttgataaaatcatgataacctgcataattttgaactaatgttaagatccacatatgtacaccttttgcggtatttataacttcataattggtgttctgaattgtattttacagataagatgatggaatatcctctgcaatatagagagaaaggtagcatagctcgtgatgctggtaagtataattaactatttggaattttcagtctgctagaacttgttctgttttacggcaaacttgcagaaaattttgaagtagaaaattgaaatttctgataaactaatggcctgaatttggacccttgctacttcctttattgaatttcttttaatatgtcctccccatttcttttcatttgatttggttagtctttggaggtccttttttgtttttttcctaggcatactaggtcatggtctttatgaattaagaaaaacaaaattcacctacttgatttaaggttctttcttggataaaccacaatggctttctttgatctatttagcccatgggaaaaggcttttgttgttattgctaccattgtcctagagttatgatgttatgccacaatttttctacctttgttttgaatgtaagctccacttttgagtgcaagagcttcttgtatactttaccatcatgtctgctggtaaaattgctactattagctgtaaaccccctccctccggcatgcacacaaacaaaaaaaaaagctaccattagaattgcagtgtatttctagattcagctgtgttgtgtgcacttctaaattgatcatatatgtatgggtagtggtgctgatatcattcatcaattatgtcccccctactggtgctcataattgccagccattttagtatctaggtaatgtgcacctgctccacttaaatatctggtgaacagaacttgttcttcgcgaatctaaatgcaatggaatatgataaaatcttgtaggttggctgtactttatcattacgggtataagaggatgatttttgttgtaataaattgtcaaaatatcaaataagtgatatacaactttaatgacatatatttatgcacccacatggtagcataatattcatcttagattgcaactatctttaagaggtacagagaattgttgtttaaacttttaaagcattgtaataagtttccatacatcaaattgttacaacttaagtattcataatatatatatatcgctttgataagatgtagagggtcaggcaagcagaaaagtgtcattgcagcgatatcttgaaaagcgtaaggacaggtatttgcagtactgaactgccatttttatagacatgtattccagccattgtgttacaagtgtatatacttttagtgatagcccgctgaattattctcttaatctgccttctatgttagggggagattgaaaggcaagaaattgactgggataacttcatctaacttcgagatgtatttgaaccttccagtgaaggtccatgcctcaaatgggaattcaagccgtagcagtactagctctccaccacagcctagactgcctctagtatctagtggttcagctgacaaccagctaaaggttgcccttcccattgatctcaatgacaaaggttggttgcaatcttctcttcaactgctaagtaacaaaacacattttttcttacaagaatgtgataattagccttcaacaagattttaaaattatattaataattgatgttaccttgtaaactgcttctaagagaaccttgtaaactgatgttaccttttatgactagtttacatataatatttgtggtggtgattaaaatcctaatgttgaatatgatgaacggtcaatctgaatgctaggtaactaaagtatttttcaatattttgtcataaatttatgttgtctcagtgtcattgcagatgttcaagaatgctaaaactctaactagatagattatgcagagttgggactcgtatatgaatgattgaagagagatgatgaaagtattataactgaagagaccatagcgtctagcagcctactgcaacaattggactgctcttagactggtcgattttagtatcttttaactgtttgggcaaatttcttttgtttaggatagtggagtgtcctgcttacctgatcaattttgtaatctaatttcatccttcgggaatttcctgttactgaagttttaaggttttatgagttgaaatgtagagccaggagcagccattttccgaggaatatatgatatgtaaattcatggcgctgcctcttgttaaatgttaatagcttttgtgcaaagccaaaccactctcttactcttctcttacgtttgcttactctcttgttgctggttttgattttctttcagcatacgaatgacttgagtaatttcattgaatgttgatattttgtaggataaatgtgttacgccataatcaataatgccaaccttattttggccgcaaacaacctttgaattgactagaacattctggtttaatttatatcttcatttaaaatattatatgttgtgaccggaaaaatatgattaattatataaatgtctaaatttattgtcatgtgatgtaaaaagtgatattctgaaattttacacatttatctgccacaaaattagacagttaacaagagggtgcaagcactcaaacaaaacgacgataacgatatttttttttttcgggtggaaacacccgc

SEQ ID NO: 32 - DNA, 6444 bp, Synthetic construct
  source 1..6444: mol_type = other DNA
agtaattgaaatgatgagagtcaatgcggggagtgaccttgtgagggccatgcgtgtggggggagtggaagcccataccgttgcaaagagcaatggtgaagtgaatcaaagtagcaaaaagcaaaagcaaggtttgagcggagcggaacgcaacgcaatgcaagtggacaaagccaatttattcacccaagaaaccccccaaccccaacaaaataaaataaatacactaaataaaccttaaccgataatattagtataatgatgtaaaattctatagcatttatataacaggagaatttatttgtatttggagagaagaagaaataaagcttatagttatagtgagaagagaaggaagattagttgctaataattaagacagaaacatacgtggcagcttagggttcaccccaagtcctaaaaacgccccctccctacccttcccccatttcccccccaacccaaacgctgccaacaacaacactctctctacaaaattgatggtcccaccgacgagcgggtgtcggaatcggaggccatgaacggcggcgccaccaccgccaccttccgatccatcctcgacaagcccctcaaccagcttaccgaggatgacatttctcagctcactcgcgaagactgtcgcagattcctcaaagaaaaaggtcccttctctttctctctcttctctcctccatgatttccggcacctgaaaaacctccattcccctaaatacattatcatattatttattaaaacattaatcgctcgctcgggatgcgggttttttaaaaattaaatttaacgagaatgatattaatagttaattaattaaaatgtcgaagacacgtgttcgtacagggatgcgtaggccttcctggaacaaatcgcaggccaacaggtcatttccctgaaagcgctgctggaaccttccgacgatgatactcctcctcctcctcctcccgccatgcaccaccgtagccatgctcaacctcaacctcaagtgagttacttactactttcttctttctttcgcctttgcttctggaacctcaaatatcctcgtgtgcttgtaggtgaatttgagtgaacctcctcctcctccgcccaaggctccgccacctgaagaacccgcttttcatgctgctgaagacattcagaaatctgcgtcgtctggggaaaagcctacggaaactaatgacaccaacaccaacgttgctagccccaagtttgtttttctctctctctctctctctcttgatggtttttaatgcctttttgtgtgaccggtatagttattttttaataatttttttaaggttttagggtgtggaatctcgtccaagctagttggatctttgatgttgctttcggtactttgtgggaagaaaatagtgtggttggtaaagtgtgctttggagttcagtgaagagtgtaggttggttaaaacttgaaacgttaaggtttgcttttttaagtggattttcttttggcattagcttggaaaattggctcattgtgttgtgttatttattattatttttttctttttcgtgaggtggaaaatgatacggacggtgggtttttggtatccgttgaatgtgtatgggtggggaaagtggaggaaaagctgttagtgaactagtcttagctcttaggtatgtttggatacatttctccataaacactttaggtgaaaaaaaatagaaaataaaatgagttggacttctactataagctaatgttaaattttatataagctttcacttttaacaatttaacttatcctaaaactgtagtgtatataggttggttttagtttatgggagaagctcaattcattttatcttcttattttcgcctatcatcagtacctataaagaagtttatcctaatagggccttgaaacagtttgtatattcctgcagtttaagaaaatgttaggtgtatttctgggatcatgggttggatctttctgactgtgaggttaggaaaaggtttatggccattaatttggatgtttggatttttctgggatgtattaggtgggttctttgttttttttagcagggttggttttattttttgggtcggggtggggagaagaatgattggaaagtgctaccttctagaagcactaaggatttctgtccaattccaaacaaccagtcagagcatgcgaagcaaatatctgttatttccaaatctagatatccccataactttaatattgttgttattctatatttgttgcattgtgtttgcactttgttgactaattaatatttaattgttgttttttgttttccattcatgtttgaccttatacgattgtgtcatactactttaatttaaaattttctctttagatatcatgattgaggtggagaaaggttcatgcggattaaatacgtggcaaatatcaaaaggaggaaggggaattctttaggtgtagaagtgattcttttaggttttaacatcatgaatttcaagattcaaggagtacaaataaataatagtgaagtttccatccataagtttgtgttggtatctatgtcaagtcttgtttccttattagtagcatccaaataaatgaacctgtttatttgtggataaccaataacccttgatttcagccatggatatgctaggaagtcatatgtcttgttgatatggaatacctgggagattttgctttcagctgcagcaagtggtaatcatacctagctgaaaagaaagctgacatgagtattataattaggtattagggacctggatgaaaaaatagagaaaagaagagaaagatttaatttcttgattaaaatgaacagaacaaaggaaaactaaatctcctccaagggtttccccttgacgaagggtttctgttttcaaaatagaactaatgctcagttttccaaatgataacaaaaacgtaaggaagcctcttcccttgtatccttttttttttatcttattatttactaaccccctctatctatctaactaactaactaactaatattccaactgtccctaactaacttccccttcccctctgccttaacgttaagtgttatagatttactcaagtgtcttcccagattcatgtttaaattaaaatcggctcttgtgcagttctttacaaaaataggagtccaggatagtaagtgtacaaaactttcattatatagttactttccgtatgaatggagcgatttggtaacctaaaatgtcagatagtttaaattcaatacatttgcataaaatatacatgactggtggcaggccagtgttccttttatgtagtattgtccattcatactcttaacattacttacaaatgtggtcatagagggtgtgcaactagtggatcatttgggcaaatgacaattttctattgtggtaaggtgaatgtctatgacagagtctcgcctgataaggttggtaatgatctatcctcttgcatcaatagctggccataatccaataagacttcaccttccaatgcaaccttatttggatttgcaggcacgagcaatcatgcagcttgcaacgagtccagtccagcttactcaggatgatcctttaaatggaaatgcagctgtttggacttctccttgccacttaccgatggataaggatgtcctcgttcctgttgatactacaatccttcaggttgctcaagcaggtagcaagtgggttcaaagattgcttgattttgctctgaagtagtagtatgaagtactttgatgtgcaaggaaatgagtcttcttcagtgcccattgcatcaaaatctggaattcttcaaggaatgatctcatctgatgagcagatgaagaaaatgattgtagcctttttattagcttaactgccggattcaatttatttcttaaaagaggatgcagtgctaagttgccttgctatgtcttacagaaaaaaatcatgaaatagggttcaagctaattgttgctttttattttactatttcatgtgccatgttcgttgagtttttcatgctattggttgttcctttgctgtgcatgttgaaaagtattttgatgtccagtcattaaaatgaccacattccctctgaatgctgattttttgaattatagcctttgagtctgtgtgtcttgaagatggacaagcataacatctttgacttgcttaaatcatgataacttgcataatttgaaattaaagatgttaagatccacatatttacttttgtgatattgctaacttcataattggtgttccgaattgttttttacagataagatggtggagtatcctctgcaatatagggagaaagggagcatagctcgtgatgctggtaagtattattaactgtttggaattttcagtctgctagaacttgttctattgtgaggaaaatttacagaaaatctcgaagtagaaaaataaatttctgataaactagtgagctgaatttggacccatgctacttcccttattgaatctctttcaacatgtcctacccatttcttttcactgatttggcaagtctttggagacccttttttgttttttttcctaggcataccaggtcattattttttttaatcaagaaaaacaaaattcgccaacttgatttaagtctctttcttggataaaacacaatggctttgtttgatcattgtagtccatggaaaaacatgcctggttaacagaacttcctgttctttgtgaatctaaaagcaatgaaatatgataaaatcttgtgggtttgatgtactatatcatcacaactttaatgtcatatttatgcacccacatggcagcataatattcatcttagattgcaagtatctttatgagttacagagaattgtttaaacttcaaagcattgtaataagttttcatacatcaaattcctagaacacaagtattcataatatatgtatatatctgtttcataagatgtagagggtcaggaacaccgaaaagtgtcattgcaacgatatcttgaaaagcgtaaggacaggtatttgcagtactaaactgccatttgtttatagacatcaatttcatccattttgttacaagtgtgtagtttagtgatagtctgctgaattattctcttaatctgccctgtatgttaggggaagattgaaaggcaagaaattgactgggataacttcatctaacttcgagatgtatttgaaccttccagtgaaggtccattcctcaaatgggaattcaagccgtagcagtactagctctccaccacaacctagactgcctctagtttccagtggttcagatcagctaaaggttgcccttcccattgatctcaatgacaaaggttagttgcaatctttcttcaacgttaagtaacaaaaccttttttttccttacaagaatgtgataattagtcttaaagcttttaagattatatcagagaaatataataattgaattgaatctgattgtcctgcttgctgtcttgatatttggggaagtgagatttgcaaaccatctgacaagcagtgggggttctttgtgtgcaatgcttccaagacaaccttataaaccggtgttaatcttttacgactagtttacaaagtataatatttgtggtggtgattaaaaattaaatggtgaaccaagatatgataaaccgtcagcctcgatactaggttaccatttgttaaacaatattttatgtcatagatttatgttgtctcagtgtctttgcagatgttcaagaatgctaaaattcaaactagatagattatgcagagttgggactcgtagatgattgaagagagatgacgaaagtattataactgaagagaccatagcgtctagcagcctactgcaagaattggattgctcttagactggtcaattttagtatatttaactgtttgggcaaatttcttttgtttaggatagtggagtgtcctgcctacctgatcaattttgtaatctaatttcatccttcgggaatatcttgttactgaagttttaaggttttatgagttgaaatgtagagccaggagcagctattttcccaggaatatatgatatgtaaattcatcgtgctgcctcttgttaaatgttaatagcttttgtgcaagccaaactattctcttactcttcttatatcttatgtttgttcactctagccaaactattctcttactcttcttatatcttatgtttgtttactctattggtgctgatttcgattttgtttcatgacaaacaactttagtaatttcactgaacgttgatattttgtaggataaatctgtcacgctatactgccattttttttttttggccacagacagttttttttaatcttttatattgatgtgaagtatttattaattttgtaatgactggaaaatttgagtttaatttatatcttaatttaaaatattatatattttttgtgacaggaaaaaacttg

SEQ ID NO: 33 - DNA, 7306 bp, Synthetic construct
  source 1..7306: mol_type = other DNA
agtgtcacgtactacatcaaaaaaaaaaaaagtgtcacatacacatataatgcaccacatcgacgaaatgctaacttcaacgaaagagaactaatggttgaactcacatgataaacaaaatatagtttcagagactattttatgaatttctttcaatccaaaaagtaacgttacaacgaataacagattcaagaattaaagcgagtaattactcttttatcattacttgcaatgacctaattgtagataggactaaagactcctaatttgaaaatatttacctgttatgttgttactcttgattggggtataatttcttattgtaaacatatttaatctaccttgcaattgggttatacattgtggatttcaacttctcatgagtctttaggttgacattgtagttaggtcgaactagatcgatctatccagaatgaattcgattatttggaggcaaaacaaaattaattttgatcaaatgaatattaattaaattattaaatataggaaaactatgtattttatgaagacgtcattttttagaaagtagtctttttaaccttaagaaaggtaatagtaattgagatgatgggagtcaatgcggggagagaccttgtgagggccatgcgtgtggggagtggaagcccataccgttgcaaagtgcaatggtgaagggaagaaaagtagcaacgcaagtggacaaaggcaatttattcacccaagaaacccccaaccccaacaacacacaaataattacaaagagacgccaacttccacgcagaaataaaagtaaataaatgaacagtaagtaaagcttaagcaatgatgttgacccaattcgaggtatttatatttatgggaataatttatttgatgaaaaaatttatatatatatatatatatattttttttttacgtaaaattcatttggatagcattaatctacgatgaaatagattaagatatctgaaatctcaaatataaaaaaaaataaaaacgtatagttataaggataagagaaggaagattagttgtgaataattaagacagaaacatacgtggcagcttagggttcaccccaagtcctaaaaaagccctcccagccttcccccatttccccccaacccaaacgctgaacaacaaccacactctctacaaaattgatggtcccaccgacgagcgggtgccggaatcggaggccatgaacggcggtgccaccaccgccaccttccgatccatcctcgacaagcccctcaaccagctcaccgaggatgacatttctcagctcactcgcgaagactgtcgcagattcctcaaagaaaaaggtctctctctctctctttctctctcttctctcctccatgatttccggcacctgataaacctccattcccctagatacattattatattataattaaaacattaatcccttgctcgctcgggatgcgggtttttttaattataaattaaatttaacgagaacgatattaatgaattaataattagtaattaaaatgtccaagacacgtgttcgtacagggatgcgcaggccttcctggaacaaaacaggtcatttccctgaaagcgctgctggaaccttccgacgatgatactcctcctcctaccgccatgcaccaccgtagtcatgctcctccccctccacctcaacctcaatctcaagtgagttactttcttcttcctttcacctttgcttctggagcctctaatattatctcatgaatcgtggttgtaggtgaatttgactgaacctcctcctccgcccaaggctccgccacctgaagaatcctcttttcatgctgctgaagacattcagaaacctgcgtcgtctggggaaaaaccttcggaaactaatgacaccaacaccaacgttgctagccccaagtttgtttctttctctctctctctctctctctctctctctctctctctccatgatttttaatgcctttttttgtgtgaccgtcatagttctttttttaatcattttttttaaggttttggggtgtggaatctcgtccaagctagttggatctttgatgttgctttcgaaactttgtgggaagaaaataatgtggttggtaaggtgtgttttggagttcagtgaagagtgaaggttgtggactagagggactagataggaggatttgggtggttaaaactttaaatgttacggtttgcttttggtgttaggttggaaaattggctcattgttttgtttcttaaaattttttgtgaggtgggagatgatacggacggtgggtttttggtatccattaattgtatatgggtgggctaagtggaggaaaagctgttggtgaactagtcttaagggtctgtttggataaatttttccataaactcttcaggagaaaaaaaagaaagaattggacttctcctataagctaatgttaaattttatagaagctttttcttttaacaatttaacttctccaaaaactgaagtgtataagttgtttttagcttatgggagaaactcaattcgttttatattcttattttttcctatacagaagtttgtcgtgggatcatgggttggtgcttaaagtgaggttagaaaaaggtttatggtcattaatttggatgtgtggatttttctgggatgtattaggtgggtttttttaggaggggggggggggggggatgattggaaagtgctacctccagaagcactatggatttctatcaattccaaacaaccagttggcgcatgggaagcaaatatctgctgtttccaaatctagatatccctgtaactttaatattgttgttattctatatttgttgcattgtgtttgcacttttgttgactaattaatatttaattgttggtttttgttttccattcatgcccaaccttatacgattgtgtcatgctttaatttaaaattttctatttagatatcatgattgaggtggagacaaggttcatgcaggttaaatatcaaaaggaaggagggaagttccttaggtgtaaaagtgattattttaggtttaatatcatgagtctcaagattctaggagtacaaacaaataagagtgaagtttccatccataaatttgtgttggtctctgtcaaggcttgttttgttatttctagcatctaaacaaatgaacctgtttatttgtggataaccaacaacccttgatttcagccatggatatggtaggaaattgtatgttatgtggatatggaatctcttggaaattttgctttcagttgcagcaagtggtaatcatacctagctgaaaagaaaactgacatgagtattataattaggtgttaggaacctggatgagaaaatagagaaaagaagagaaagattgtatttcttgattaaaaatgaacaaggcaaaggaaagctaaatctcctccaagggtttccccttgacgaagggtttccatttccaatagagctaatgctcagttttccagatgataacaaaacagaatgaagccacttcccttgcatccttctatttatttaccttactaacctcatctaactaactaatattcatgtttaaattaaaaactgcccttgtgccattctttacaaaaataggagtctaggatagtgattaactgttcaaaacattctttacatagttactttccctatgaatagagcgatctggtaacctaaaatgtcaattagcttagattcaatacatttggcattaaaaatacgtgactagttgctggccagtgtttcttttatgtattgtccatttgtactcttaacattacttacaaatgtgatcacagagggtgtgcaactagtggatcatttgggcaaatgacaattttctattgtggtaaggtgaatgtctatgatggagtctcgcctgataaggttggtaatgatccatcctcttgcataaatagttggccataaatccaataagatttcacctttcaatgcaatgtatttggatttgcaggcacgagcaatcatgcagcttgcggtgagtcctgtccagtttactcaagatgatccttcaaatggaaatgcagctgtttggccttctccttgccacttaccaatggataaggatgtcctcattcctgtagatacaacaatccttcaggttgctcaatcaggtagcaagtgggttgaaagattgcttaattttgctgaagtattggtactttgatgtgcaacggaatgagtcttcttcagtgcccattgcataaaaaattggaattcaaggaatgatttcatctgatgaacagatgaaggaaaatattgtagtctttttattagtttaactatcctgattcaatatatttcttaaaggaggatacaatgccaagttgccttgctttgaaatcatgaaatagggttgaagctaatagttgctttttgttgaactatttcatgtgtcatgttcgttgagtttttcatgctactggttgttccgttgctgtgaaagttgaaaaataattggatgtccagtcattattatgatcaaattcactctgaatgcctaatattagaattatagcttttgagtctgtgtcttgaagatggacaggcacaacatctctgacttgataaaatcatgataacctgcataattttgaactaatgttaagatccacatatgtacaccttttgcggtatttataacttcataattggtgttctgaattgtattttacagataagatgatggaatatcctctgcaatatagagagaaaggtagcatagctcgtgatgctggtaagtataattaactatttggaattttcagtctgctagaacttgttctgttttacggcaaacttgcagaaaattttgaagtagaaaattgaaatttctgataaactaatggcctgaatttggacccttgctacttcctttattgaatttcttttaatatgtcctccccatttcttttcatttgatttggttagtctttggaggtccttttttgtttttttcctaggcatactaggtcatggtctttatgaattaagaaaaacaaaattcacctacttgatttaaggttctttcttggataaaccacaatggctttctttgatctatttagcccatgggaaaaggcttttgttgttattgctaccattgtcctagagttatgatgttatgccacaatttttctacctttgttttgaatgtaagctccacttttgagtgcaagagcttcttgtatactttaccatcatgtctgctggtaaaattgctactattagctgtaaaccccctccctccggcatgcacacaaacaaaaaaaaaagctaccattagaattgcagtgtatttctagattcagctgtgttgtgtgcacttctaaattgatcatatatgtatgggtagtggtgctgatatcattcatcaattatgtcccccctactggtgctcataattgccagccattttagtatctaggtaatgtgcacctgctccacttaaatatctggtgaacagaacttgttcttcgcgaatctaaatgcaatggaatatgataaaatcttgtaggttggctgtactttatcattacgggtataagaggatgatttttgttgtaataaattgtcaaaatatcaaataagtgatatacaactttaatgacatatatttatgcacccacatggtagcataatattcatcttagattgcaactatctttaagaggtacagagaattgttgtttaaacttttaaagcattgtaataagtttccatacatcaaattgttacaacttaagtattcataatatatatatatcgctttgataagatgtagagggtcaggcaagcagaaaagtgtcattgcagcgatatcttgaaaagcgtaaggacaggtatttgcagtactgaactgccatttttatagacatgtattccagccattgtgttacaagtgtatatacttttagtgatagcccgctgaattattctcttaatctgccttctatgttagggggagattgaaaggcaagaaattgactgggataacttcatctaacttcgagatgtatttgaaccttccagtgaaggtccatgcctcaaatgggaattcaagccgtagcagtactagctctccaccacagcctagactgcctctagtatctagtggttcagctgacaaccagctaaaggttgcccttcccattgatctcaatgacaaaggttggttgcaatcttctcttcaactgctaagtaacaaaacacattttttcttacaagaatgtgataattagccttcaacaagattttaaaattatattaataattgatgttaccttgtaaactgcttctaagagaaccttgtaaactgatgttaccttttatgactagtttacatataatatttgtggtggtgattaaaatcctaatgttgaatatgatgaacggtcaatctgaatgctaggtaactaaagtatttttcaatattttgtcataaatttatgttgtctcagtgtcattgcagatgttcaagaatgctaaaactctaactagatagattatgcagagttgggactcgtatatgaatgattgaagagagatgatgaaagtattataactgaagagaccatagcgtctagcagcctactgcaacaattggactgctcttagactggtcgattttagtatcttttaactgtttgggcaaatttcttttgtttaggatagtggagtgtcctgcttacctgatcaattttgtaatctaatttcatccttcgggaatttcctgttactgaagttttaaggttttatgagttgaaatgtagagccaggagcagccattttccgaggaatatatgatatgtaaattcatggcgctgcctcttgttaaatgttaatagcttttgtgcaaagccaaaccactctcttactcttctcttacgtttgcttactctcttgttgctggttttgattttctttcagcatacgaatgacttgagtaatttcattgaatgttgatattttgtaggataaatgtgttacgccataatcaataatgccaaccttattttggccgcaaacaacctttgaattgactagaacattctggtttaatttatatcttcatttaaaatattatatgttgtgaccggaaaaatatgattaattatataaatgtctaaatttattgtcatgtgatgtaaaaagtgatattctgaaattttacacatttatctgccacaaaattagacagttaacaagagggtgcaagcactcaaacaaaacgacgataacgatatttttttttttcgggtggaaacacccgc

SEQ ID NO: 34 - DNA, 7294 bp, Synthetic construct
  source 1..7294: mol_type = other DNA
agtgtcacgtactacatcaaaaaaaaaaaaagtgtcacatacacatataatgcaccacatcgacgaaatgctaacttcaacgaaagagaactaatggttgaactcacatgataaacaaaatatagtttcagagactattttatgaatttctttcaatccaaaaagtaacgttacaacgaataacagattcaagaattaaagcgagtaattactcttttatcattacttgcaatgacctaattgtagataggactaaagactcctaatttgaaaatatttacctgttatgttgttactcttgattggggtataatttcttattgtaaacatatttaatctaccttgcaattgggttatacattgtggatttcaacttctcatgagtctttaggttgacattgtagttaggtcgaactagatcgatctatccagaatgaattcgattatttggaggcaaaacaaaattaattttgatcaaatgaatattaattaaattattaaatataggaaaactatgtattttatgaagacgtcattttttagaaagtagtctttttaaccttaagaaaggtaatagtaattgagatgatgggagtcaatgcggggagagaccttgtgagggccatgcgtgtggggagtggaagcccataccgttgcaaagtgcaatggtgaagggaagaaaagtagcaacgcaagtggacaaaggcaatttattcacccaagaaacccccaaccccaacaacacacaaataattacaaagagacgccaacttccacgcagaaataaaagtaaataaatgaacagtaagtaaagcttaagcaatgatgttgacccaattcgaggtatttatatttatgggaataatttatttgatgaaaaaatttatatatatatatatatatattttttttttacgtaaaattcatttggatagcattaatctacgatgaaatagattaagatatctgaaatctcaaatataaaaaaaaataaaaacgtatagttataaggataagagaaggaagattagttgtgaataattaagacagaaacatacgtggcagcttagggttcaccccaagtcctaaaaaagccctcccagccttcccccatttccccccaacccaaacgctgaacaacaaccacactctctacaaaattgatggtcccaccgacgagcgggtgccggaatcggaggccatgaacggcggtgccaccaccgccaccttccgatccatcctcgacaagcccctcaaccagctcaccgaggatgacatttctcagctcactcgcgaagactgtcgcagattcctcaaagaaaaaggtctctctctctctctttctctctcttctctcctccatgatttccggcacctgataaacctccattcccctagatacattattatattataattaaaacattaatcccttgctcgctcgggatgcgggtttttttaattataaattaaatttaacgagaacgatattaatgaattaataattagtaattaaaatgtccaagacacgtgttcgtacagggatgcgcaggccttcctggaacaaatcgcaaagcgctgctggaaccttccgacgatgatactcctcctcctaccgccatgcaccaccgtagtcatgctcctccccctccacctcaacctcaatctcaagtgagttactttcttcttcctttcacctttgcttctggagcctctaatattatctcatgaatcgtggttgtaggtgaatttgactgaacctcctcctccgcccaaggctccgccacctgaagaatcctcttttcatgctgctgaagacattcagaaacctgcgtcgtctggggaaaaaccttcggaaactaatgacaccaacaccaacgttgctagccccaagtttgtttctttctctctctctctctctctctctctctctctctctctccatgatttttaatgcctttttttgtgtgaccgtcatagttctttttttaatcattttttttaaggttttggggtgtggaatctcgtccaagctagttggatctttgatgttgctttcgaaactttgtgggaagaaaataatgtggttggtaaggtgtgttttggagttcagtgaagagtgaaggttgtggactagagggactagataggaggatttgggtggttaaaactttaaatgttacggtttgcttttggtgttaggttggaaaattggctcattgttttgtttcttaaaattttttgtgaggtgggagatgatacggacggtgggtttttggtatccattaattgtatatgggtgggctaagtggaggaaaagctgttggtgaactagtcttaagggtctgtttggataaatttttccataaactcttcaggagaaaaaaaagaaagaattggacttctcctataagctaatgttaaattttatagaagctttttcttttaacaatttaacttctccaaaaactgaagtgtataagttgtttttagcttatgggagaaactcaattcgttttatattcttattttttcctatacagaagtttgtcgtgggatcatgggttggtgcttaaagtgaggttagaaaaaggtttatggtcattaatttggatgtgtggatttttctgggatgtattaggtgggtttttttaggaggggggggggggggggatgattggaaagtgctacctccagaagcactatggatttctatcaattccaaacaaccagttggcgcatgggaagcaaatatctgctgtttccaaatctagatatccctgtaactttaatattgttgttattctatatttgttgcattgtgtttgcacttttgttgactaattaatatttaattgttggtttttgttttccattcatgcccaaccttatacgattgtgtcatgctttaatttaaaattttctatttagatatcatgattgaggtggagacaaggttcatgcaggttaaatatcaaaaggaaggagggaagttccttaggtgtaaaagtgattattttaggtttaatatcatgagtctcaagattctaggagtacaaacaaataagagtgaagtttccatccataaatttgtgttggtctctgtcaaggcttgttttgttatttctagcatctaaacaaatgaacctgtttatttgtggataaccaacaacccttgatttcagccatggatatggtaggaaattgtatgttatgtggatatggaatctcttggaaattttgctttcagttgcagcaagtggtaatcatacctagctgaaaagaaaactgacatgagtattataattaggtgttaggaacctggatgagaaaatagagaaaagaagagaaagattgtatttcttgattaaaaatgaacaaggcaaaggaaagctaaatctcctccaagggtttccccttgacgaagggtttccatttccaatagagctaatgctcagttttccagatgataacaaaacagaatgaagccacttcccttgcatccttctatttatttaccttactaacctcatctaactaactaatattcatgtttaaattaaaaactgcccttgtgccattctttacaaaaataggagtctaggatagtgattaactgttcaaaacattctttacatagttactttccctatgaatagagcgatctggtaacctaaaatgtcaattagcttagattcaatacatttggcattaaaaatacgtgactagttgctggccagtgtttcttttatgtattgtccatttgtactcttaacattacttacaaatgtgatcacagagggtgtgcaactagtggatcatttgggcaaatgacaattttctattgtggtaaggtgaatgtctatgatggagtctcgcctgataaggttggtaatgatccatcctcttgcataaatagttggccataaatccaataagatttcacctttcaatgcaatgtatttggatttgcaggcacgagcaatcatgcagcttgcggtgagtcctgtccagtttactcaagatgatccttcaaatggaaatgcagctgtttggccttctccttgccacttaccaatggataaggatgtcctcattcctgtagatacaacaatccttcaggttgctcaatcaggtagcaagtgggttgaaagattgcttaattttgctgaagtattggtactttgatgtgcaacggaatgagtcttcttcagtgcccattgcataaaaaattggaattcaaggaatgatttcatctgatgaacagatgaaggaaaatattgtagtctttttattagtttaactatcctgattcaatatatttcttaaaggaggatacaatgccaagttgccttgctttgaaatcatgaaatagggttgaagctaatagttgctttttgttgaactatttcatgtgtcatgttcgttgagtttttcatgctactggttgttccgttgctgtgaaagttgaaaaataattggatgtccagtcattattatgatcaaattcactctgaatgcctaatattagaattatagcttttgagtctgtgtcttgaagatggacaggcacaacatctctgacttgataaaatcatgataacctgcataattttgaactaatgttaagatccacatatgtacaccttttgcggtatttataacttcataattggtgttctgaattgtattttacagataagatgatggaatatcctctgcaatatagagagaaaggtagcatagctcgtgatgctggtaagtataattaactatttggaattttcagtctgctagaacttgttctgttttacggcaaacttgcagaaaattttgaagtagaaaattgaaatttctgataaactaatggcctgaatttggacccttgctacttcctttattgaatttcttttaatatgtcctccccatttcttttcatttgatttggttagtctttggaggtccttttttgtttttttcctaggcatactaggtcatggtctttatgaattaagaaaaacaaaattcacctacttgatttaaggttctttcttggataaaccacaatggctttctttgatctatttagcccatgggaaaaggcttttgttgttattgctaccattgtcctagagttatgatgttatgccacaatttttctacctttgttttgaatgtaagctccacttttgagtgcaagagcttcttgtatactttaccatcatgtctgctggtaaaattgctactattagctgtaaaccccctccctccggcatgcacacaaacaaaaaaaaaagctaccattagaattgcagtgtatttctagattcagctgtgttgtgtgcacttctaaattgatcatatatgtatgggtagtggtgctgatatcattcatcaattatgtcccccctactggtgctcataattgccagccattttagtatctaggtaatgtgcacctgctccacttaaatatctggtgaacagaacttgttcttcgcgaatctaaatgcaatggaatatgataaaatcttgtaggttggctgtactttatcattacgggtataagaggatgatttttgttgtaataaattgtcaaaatatcaaataagtgatatacaactttaatgacatatatttatgcacccacatggtagcataatattcatcttagattgcaactatctttaagaggtacagagaattgttgtttaaacttttaaagcattgtaataagtttccatacatcaaattgttacaacttaagtattcataatatatatatatcgctttgataagatgtagagggtcaggcaagcagaaaagtgtcattgcagcgatatcttgaaaagcgtaaggacaggtatttgcagtactgaactgccatttttatagacatgtattccagccattgtgttacaagtgtatatacttttagtgatagcccgctgaattattctcttaatctgccttctatgttagggggagattgaaaggcaagaaattgactgggataacttcatctaacttcgagatgtatttgaaccttccagtgaaggtccatgcctcaaatgggaattcaagccgtagcagtactagctctccaccacagcctagactgcctctagtatctagtggttcagctgacaaccagctaaaggttgcccttcccattgatctcaatgacaaaggttggttgcaatcttctcttcaactgctaagtaacaaaacacattttttcttacaagaatgtgataattagccttcaacaagattttaaaattatattaataattgatgttaccttgtaaactgcttctaagagaaccttgtaaactgatgttaccttttatgactagtttacatataatatttgtggtggtgattaaaatcctaatgttgaatatgatgaacggtcaatctgaatgctaggtaactaaagtatttttcaatattttgtcataaatttatgttgtctcagtgtcattgcagatgttcaagaatgctaaaactctaactagatagattatgcagagttgggactcgtatatgaatgattgaagagagatgatgaaagtattataactgaagagaccatagcgtctagcagcctactgcaacaattggactgctcttagactggtcgattttagtatcttttaactgtttgggcaaatttcttttgtttaggatagtggagtgtcctgcttacctgatcaattttgtaatctaatttcatccttcgggaatttcctgttactgaagttttaaggttttatgagttgaaatgtagagccaggagcagccattttccgaggaatatatgatatgtaaattcatggcgctgcctcttgttaaatgttaatagcttttgtgcaaagccaaaccactctcttactcttctcttacgtttgcttactctcttgttgctggttttgattttctttcagcatacgaatgacttgagtaatttcattgaatgttgatattttgtaggataaatgtgttacgccataatcaataatgccaaccttattttggccgcaaacaacctttgaattgactagaacattctggtttaatttatatcttcatttaaaatattatatgttgtgaccggaaaaatatgattaattatataaatgtctaaatttattgtcatgtgatgtaaaaagtgatattctgaaattttacacatttatctgccacaaaattagacagttaacaagagggtgcaagcactcaaacaaaacgacgataacgatatttttttttttcgggtggaaacacccgc

SEQ ID NO: 35 - DNA, 7316 bp, Synthetic construct
  source 1..7316: mol_type = other DNA
agtgtcacgtactacatcaaaaaaaaaaaaagtgtcacatacacatataatgcaccacatcgacgaaatgctaacttcaacgaaagagaactaatggttgaactcacatgataaacaaaatatagtttcagagactattttatgaatttctttcaatccaaaaagtaacgttacaacgaataacagattcaagaattaaagcgagtaattactcttttatcattacttgcaatgacctaattgtagataggactaaagactcctaatttgaaaatatttacctgttatgttgttactcttgattggggtataatttcttattgtaaacatatttaatctaccttgcaattgggttatacattgtggatttcaacttctcatgagtctttaggttgacattgtagttaggtcgaactagatcgatctatccagaatgaattcgattatttggaggcaaaacaaaattaattttgatcaaatgaatattaattaaattattaaatataggaaaactatgtattttatgaagacgtcattttttagaaagtagtctttttaaccttaagaaaggtaatagtaattgagatgatgggagtcaatgcggggagagaccttgtgagggccatgcgtgtggggagtggaagcccataccgttgcaaagtgcaatggtgaagggaagaaaagtagcaacgcaagtggacaaaggcaatttattcacccaagaaacccccaaccccaacaacacacaaataattacaaagagacgccaacttccacgcagaaataaaagtaaataaatgaacagtaagtaaagcttaagcaatgatgttgacccaattcgaggtatttatatttatgggaataatttatttgatgaaaaaatttatatatatatatatatatattttttttttacgtaaaattcatttggatagcattaatctacgatgaaatagattaagatatctgaaatctcaaatataaaaaaaaataaaaacgtatagttataaggataagagaaggaagattagttgtgaataattaagacagaaacatacgtggcagcttagggttcaccccaagtcctaaaaaagccctcccagccttcccccatttccccccaacccaaacgctgaacaacaaccacactctctacaaaattgatggtcccaccgacgagcgggtgccggaatcggaggccatgaacggcggtgccaccaccgccaccttccgatccatcctcgacaagcccctcaaccagctcaccgaggatgacatttctcagctcactcgcgaagactgtcgcagattcctcaaagaaaaaggtctctctctctctctttctctctcttctctcctccatgatttccggcacctgataaacctccattcccctagatacattattatattataattaaaacattaatcccttgctcgctcgggatgcgggtttttttaattataaattaaatttaacgagaacgatattaatgaattaataattagtaattaaaatgtccaagacacgtgttcgtacagggatgcgcaggccttcctggaacaaatcgcaggcgatcaggtcatttccctgaaagcgctgctggaaccttccgacgatgatactcctcctcctaccgccatgcaccaccgtagtcatgctcctccccctccacctcaacctcaatctcaagtgagttactttcttcttcctttcacctttgcttctggagcctctaatattatctcatgaatcgtggttgtaggtgaatttgactgaacctcctcctccgcccaaggctccgccacctgaagaatcctcttttcatgctgctgaagacattcagaaacctgcgtcgtctggggaaaaaccttcggaaactaatgacaccaacaccaacgttgctagccccaagtttgtttctttctctctctctctctctctctctctctctctctctctccatgatttttaatgcctttttttgtgtgaccgtcatagttctttttttaatcattttttttaaggttttggggtgtggaatctcgtccaagctagttggatctttgatgttgctttcgaaactttgtgggaagaaaataatgtggttggtaaggtgtgttttggagttcagtgaagagtgaaggttgtggactagagggactagataggaggatttgggtggttaaaactttaaatgttacggtttgcttttggtgttaggttggaaaattggctcattgttttgtttcttaaaattttttgtgaggtgggagatgatacggacggtgggtttttggtatccattaattgtatatgggtgggctaagtggaggaaaagctgttggtgaactagtcttaagggtctgtttggataaatttttccataaactcttcaggagaaaaaaaagaaagaattggacttctcctataagctaatgttaaattttatagaagctttttcttttaacaatttaacttctccaaaaactgaagtgtataagttgtttttagcttatgggagaaactcaattcgttttatattcttattttttcctatacagaagtttgtcgtgggatcatgggttggtgcttaaagtgaggttagaaaaaggtttatggtcattaatttggatgtgtggatttttctgggatgtattaggtgggtttttttaggaggggggggggggggggatgattggaaagtgctacctccagaagcactatggatttctatcaattccaaacaaccagttggcgcatgggaagcaaatatctgctgtttccaaatctagatatccctgtaactttaatattgttgttattctatatttgttgcattgtgtttgcacttttgttgactaattaatatttaattgttggtttttgttttccattcatgcccaaccttatacgattgtgtcatgctttaatttaaaattttctatttagatatcatgattgaggtggagacaaggttcatgcaggttaaatatcaaaaggaaggagggaagttccttaggtgtaaaagtgattattttaggtttaatatcatgagtctcaagattctaggagtacaaacaaataagagtgaagtttccatccataaatttgtgttggtctctgtcaaggcttgttttgttatttctagcatctaaacaaatgaacctgtttatttgtggataaccaacaacccttgatttcagccatggatatggtaggaaattgtatgttatgtggatatggaatctcttggaaattttgctttcagttgcagcaagtggtaatcatacctagctgaaaagaaaactgacatgagtattataattaggtgttaggaacctggatgagaaaatagagaaaagaagagaaagattgtatttcttgattaaaaatgaacaaggcaaaggaaagctaaatctcctccaagggtttccccttgacgaagggtttccatttccaatagagctaatgctcagttttccagatgataacaaaacagaatgaagccacttcccttgcatccttctatttatttaccttactaacctcatctaactaactaatattcatgtttaaattaaaaactgcccttgtgccattctttacaaaaataggagtctaggatagtgattaactgttcaaaacattctttacatagttactttccctatgaatagagcgatctggtaacctaaaatgtcaattagcttagattcaatacatttggcattaaaaatacgtgactagttgctggccagtgtttcttttatgtattgtccatttgtactcttaacattacttacaaatgtgatcacagagggtgtgcaactagtggatcatttgggcaaatgacaattttctattgtggtaaggtgaatgtctatgatggagtctcgcctgataaggttggtaatgatccatcctcttgcataaatagttggccataaatccaataagatttcacctttcaatgcaatgtatttggatttgcaggcacgagcaatcatgcagcttgcggtgagtcctgtccagtttactcaagatgatccttcaaatggaaatgcagctgtttggccttctccttgccacttaccaatggataaggatgtcctcattcctgtagatacaacaatccttcaggttgctcaatcaggtagcaagtgggttgaaagattgcttaattttgctgaagtattggtactttgatgtgcaacggaatgagtcttcttcagtgcccattgcataaaaaattggaattcaaggaatgatttcatctgatgaacagatgaaggaaaatattgtagtctttttattagtttaactatcctgattcaatatatttcttaaaggaggatacaatgccaagttgccttgctttgaaatcatgaaatagggttgaagctaatagttgctttttgttgaactatttcatgtgtcatgttcgttgagtttttcatgctactggttgttccgttgctgtgaaagttgaaaaataattggatgtccagtcattattatgatcaaattcactctgaatgcctaatattagaattatagcttttgagtctgtgtcttgaagatggacaggcacaacatctctgacttgataaaatcatgataacctgcataattttgaactaatgttaagatccacatatgtacaccttttgcggtatttataacttcataattggtgttctgaattgtattttacagataagatgatggaatatcctctgcaatatagagagaaaggtagcatagctcgtgatgctggtaagtataattaactatttggaattttcagtctgctagaacttgttctgttttacggcaaacttgcagaaaattttgaagtagaaaattgaaatttctgataaactaatggcctgaatttggacccttgctacttcctttattgaatttcttttaatatgtcctccccatttcttttcatttgatttggttagtctttggaggtccttttttgtttttttcctaggcatactaggtcatggtctttatgaattaagaaaaacaaaattcacctacttgatttaaggttctttcttggataaaccacaatggctttctttgatctatttagcccatgggaaaaggcttttgttgttattgctaccattgtcctagagttatgatgttatgccacaatttttctacctttgttttgaatgtaagctccacttttgagtgcaagagcttcttgtatactttaccatcatgtctgctggtaaaattgctactattagctgtaaaccccctccctccggcatgcacacaaacaaaaaaaaaagctaccattagaattgcagtgtatttctagattcagctgtgttgtgtgcacttctaaattgatcatatatgtatgggtagtggtgctgatatcattcatcaattatgtcccccctactggtgctcataattgccagccattttagtatctaggtaatgtgcacctgctccacttaaatatctggtgaacagaacttgttcttcgcgaatctaaatgcaatggaatatgataaaatcttgtaggttggctgtactttatcattacgggtataagaggatgatttttgttgtaataaattgtcaaaatatcaaataagtgatatacaactttaatgacatatatttatgcacccacatggtagcataatattcatcttagattgcaactatctttaagaggtacagagaattgttgtttaaacttttaaagcattgtaataagtttccatacatcaaattgttacaacttaagtattcataatatatatatatcgctttgataagatgtagagggtcaggcaagcagaaaagtgtcattgcagcgatatcttgaaaagcgtaaggacaggtatttgcagtactgaactgccatttttatagacatgtattccagccattgtgttacaagtgtatatacttttagtgatagcccgctgaattattctcttaatctgccttctatgttagggggagattgaaaggcaagaaattgactgggataacttcatctaacttcgagatgtatttgaaccttccagtgaaggtccatgcctcaaatgggaattcaagccgtagcagtactagctctccaccacagcctagactgcctctagtatctagtggttcagctgacaaccagctaaaggttgcccttcccattgatctcaatgacaaaggttggttgcaatcttctcttcaactgctaagtaacaaaacacattttttcttacaagaatgtgataattagccttcaacaagattttaaaattatattaataattgatgttaccttgtaaactgcttctaagagaaccttgtaaactgatgttaccttttatgactagtttacatataatatttgtggtggtgattaaaatcctaatgttgaatatgatgaacggtcaatctgaatgctaggtaactaaagtatttttcaatattttgtcataaatttatgttgtctcagtgtcattgcagatgttcaagaatgctaaaactctaactagatagattatgcagagttgggactcgtatatgaatgattgaagagagatgatgaaagtattataactgaagagaccatagcgtctagcagcctactgcaacaattggactgctcttagactggtcgattttagtatcttttaactgtttgggcaaatttcttttgtttaggatagtggagtgtcctgcttacctgatcaattttgtaatctaatttcatccttcgggaatttcctgttactgaagttttaaggttttatgagttgaaatgtagagccaggagcagccattttccgaggaatatatgatatgtaaattcatggcgctgcctcttgttaaatgttaatagcttttgtgcaaagccaaaccactctcttactcttctcttacgtttgcttactctcttgttgctggttttgattttctttcagcatacgaatgacttgagtaatttcattgaatgttgatattttgtaggataaatgtgttacgccataatcaataatgccaaccttattttggccgcaaacaacctttgaattgactagaacattctggtttaatttatatcttcatttaaaatattatatgttgtgaccggaaaaatatgattaattatataaatgtctaaatttattgtcatgtgatgtaaaaagtgatattctgaaattttacacatttatctgccacaaaattagacagttaacaagagggtgcaagcactcaaacaaaacgacgataacgatatttttttttttcgggtggaaacacccgc

SEQ ID NO: 36 - DNA, 23 bp, Synthetic construct
  source 1..23: mol_type = other DNA
agggaaatgacctgttggatcgc

SEQ ID NO: 37 - protein, 801 aa, Acidaminococcus sp.
MEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 38 - DNA, 30 bp, Synthetic construct
  source 1..30: mol_type = other DNA
aacaaatcgcaggcgacaggtcatttccct

SEQ ID NO: 39 - DNA, 27 bp, Glycine max
  source 1..27: mol_type = genomic DNA
gcgatccaacaggtcatttccctgaaa

SEQ ID NO: 40 - DNA, 27 bp, Glycine max
  source 1..27: mol_type = genomic DNA
tttcagggaaatgacctgttggatcgc

(19) United States
(12) Patent Application Publication
(10) Pub. No.: US 2024/0301012 A1
(43) Pub. Date: Sep. 12, 2024

(54) SOYBEANS WITH COMBINATIONS OF GENE MUTATIONS

(71) Applicant: INARI AGRICULTURE TECHNOLOGY, INC., Cambridge, MA (US)

(72) Inventors: CHRISTINE MARIE GAULT, CAMBRIDGE, MA (US); Zachary Lemmon, Natick, MA (US); STEVEN D. ROUNSLEY, Waunakee, WI (US); RODRIGO GERMAN SALA, Wildwood, MO (US); Randall William Shultz, Davis, CA (US); RACHEL ANNE MERTZ, West Lafayette, IN (US); AMANDA PEREIRA DE SOUZA, West Lafayette, IN (US)

(21) Appl. No.: 18/606,199

(22) Filed: Mar. 15, 2024

Related U.S. Application Data
(63) Continuation-in-part of application No. PCT/US24/10705, filed on Jan. 8, 2024.
(60) Provisional application No. 63/490,375, filed on Mar. 15, 2023.
(60) Provisional application No. 63/479,312, filed on Jan. 10, 2023.

(51) Int. Cl. C07K 14/415; C12N 15/82

(52) U.S. Cl. CPC C07K 14/415; C12N 15/8274; C12N 15/8279

(57) ABSTRACT

The disclosure relates to novel soybean plants, plant parts, and nucleotide sequences in soybean plants comprising a mutated FT1a and other genes, along with methods of using and making the same.

### CROSS REFERENCE TO RELATED APPLICATIONS

This U.S. non-provisional patent application claims the benefit of U.S. provisional patent application Ser. No. 63/490,375, filed Mar. 15, 2023, and is a bypass continuation-in-part of international patent application PCT/US24/10705, filed Jan. 8, 2024, which claims the benefit of U.S. provisional patent application Ser. No. 63/479,312, filed Jan. 10, 2023, the entire contents of which including the specification, claims, figures, and sequence listing are incorporated herein by reference in their entireties.

### SEQUENCE LISTING XML

The instant application contains a sequence listing, which has been submitted in XML file format by electronic submission and is hereby incorporated by reference in its entirety. The XML file, created on Mar. 14, 2024, is named P14293US01.xml and is 119,772 bytes in size.

### TECHNICAL FIELD

Disclosed herein are novel plants, plant parts, and nucleotide sequences in soybean varieties comprising a mutated FT1a, JAG1, BS1, BS2, and/or TFL1b gene, along with methods of making the same by growing a soybean plant or lot, and methods of using the same.

### BACKGROUND

Agriculture is an essential industry for the global economy and the United States in particular. Soybean (Glycine max) is an important legume crop worldwide due to its ability to fix atmospheric nitrogen. Soybeans serve as a major source of animal feed protein and soybean oil has uses in a wide variety of industries, including the food and beverage, biodiesel, and other industries.

Soybean sustainability is a priority for farmers worldwide. Farming practices such as water and nutrient management help farmers improve efficiencies, boost crop productivity, conserve water, enrich soil quality, improve nutrient efficiencies of the soil, and produce sustainable soybean crops. The benefits of bioengineering for soybean farmers include increased yields and extreme weather hardiness.

### SUMMARY

Disclosed herein are soybean plant cells comprising loss-of-function allele(s) of the endogenous soybean FT1a gene of SEQ ID NO: 3, JAG1 gene of SEQ ID NO: 18, BS1 gene of SEQ ID NO: 25, BS2 gene of SEQ ID NO: 26, TFL1b gene of SEQ ID NO: 22, and/or an allelic variant thereof. Also provided are soybean plant parts comprising the aforementioned soybean plant cell, including stems, roots, leaves, flowers, pods, and seeds. Also provided are soybean seed lots comprising the seed. Also provided are soybean plants comprising the aforementioned soybean plant cells.

Also provided are biological samples comprising a nucleic acid containing loss-of-function allele(s) of the soybean FT1a gene of SEQ ID NO: 3, JAG1 gene of SEQ ID NO: 18, BS1 gene of SEQ ID NO: 25, BS2 gene of SEQ ID NO: 26, TFL1b gene of SEQ ID NO: 22, and/or an allelic variant thereof. Polynucleotides comprising the loss-of-function allele of the soybean FT1a gene set forth in SEQ ID NO: 4, SEQ ID NO: 5, SEQ ID NO: 7, and SEQ ID NO: 8 are provided. Polynucleotides encoding the polypeptides of SEQ ID NO: 6, SEQ ID NO: 9, and SEQ ID NO: 10 are also provided. In some embodiments, the aforementioned polynucleotides are isolated.

Also disclosed are methods of producing a soybean seed lot comprising: (i) growing a population of soybean plants comprising the aforementioned soybean plant; and (ii) harvesting seed from the population of soybean plants of step (i) at maturity. Methods of producing a soybean crop comprising planting the aforementioned seed lot are provided.

Guide RNA molecules comprising a spacer RNA molecule which target exon 1 of the FT1a gene of SEQ ID NO: 3 or an allelic variant thereof are provided. Guide RNA molecules comprising a spacer RNA encoded by SEQ ID NO: 11 are also provided.

Also disclosed are methods for generating the aforementioned soybean plant cells, soybean plant parts, and soybean plants are provided. In some embodiments, the methods comprise introducing loss-of-function allele(s) in the endogenous soybean FT1a gene of SEQ ID NO: 3, JAG1 gene of SEQ ID NO: 18, BS1 gene of SEQ ID NO: 25, BS2 gene of SEQ ID NO: 26, TFL1b gene of SEQ ID NO: 22, and/or an allelic variant thereof. In some embodiments, the methods comprise (i) screening a population of soybean plant cells, parts, or plants for the presence of a loss-of-function allele in the endogenous soybean FT1a gene of SEQ ID NO: 3 or an allelic variant thereof; and (ii) isolating a soybean plant cell, soybean plant part, or soybean plant comprising the loss-of-function allele of the soybean FT1a gene of SEQ ID NO: 3 or an allelic variant thereof.

Methods for determining whether a soybean plant cell, plant part, or plant comprises a loss-of-function allele of the endogenous soybean FT1a gene of SEQ ID NO: 3, JAG1 gene of SEQ ID NO: 18, BS1 gene of SEQ ID NO: 25, BS2 gene of SEQ ID NO: 26, TFL1b gene of SEQ ID NO: 22, and/or an allelic variant thereof are provided. In certain embodiments, the methods comprise analyzing a polynucleotide comprising a portion of SEQ ID NO: 3 or an allelic variant thereof or analyzing an RNA encoded by a portion of SEQ ID NO: 3 or an allelic variant thereof from the plant cell, plant part, or plant, wherein an insertion, deletion, and/or substitution of one or more nucleotides in said polynucleotide or RNA is indicative of the presence of the loss-of-function allele. In certain embodiments, the methods comprise analyzing a polypeptide encoded by SEQ ID NO: 3, a portion thereof, or an allelic variant thereof from the soybean plant cell, plant part, or plant, wherein an insertion, deletion, and/or substitution of one or more amino acid residues of the polypeptide or a change in the biologic or biochemical activity of the polypeptide is indicative of the presence of the loss-of-function allele.

### DESCRIPTION OF THE DRAWINGS

FIG. 1A, B show the wild-type FT1a gene (SEQ ID NO: 3) with the exons of the mRNA splice variant 1 (Glyma.18G298900.1) transcript that encode the FT1a protein of SEQ ID NO: 1. All exons are in bold, the translated bases (codons) are in upper case, introns are in lower case, and both the 5′ and 3′ untranslated regions (UTRs) are in lower case and underlined.

FIG. 2A, B show the wild-type FT1a gene (SEQ ID NO: 3) with the exons of the mRNA splice variant 2 (Glyma.18G298900.3) transcript that encode the FT1a protein of SEQ ID NO: 2. All exons are in bold, the translated bases (codons) are in upper case, introns are in lower case, and both the 5′ and 3′ untranslated regions (UTRs) are in lower case and underlined.

FIG. 3A, B show an ft1a gene mutant (SEQ ID NO: 5) with the exons of the transcript that encode the mutant ft1a protein of SEQ ID NO: 6. All exons are in bold, the translated bases (codons) are in upper case, introns are in lower case, and both the 5′ and 3′ untranslated regions (UTRs) are in lower case and underlined.

FIG. 4A, B show an ft1a gene mutant (SEQ ID NO: 8) with the exons of the transcript that encode the mutant ft1a protein of SEQ ID NO: 9. All exons are in bold, the translated bases (codons) are in upper case, introns are in lower case, and both the 5′ and 3′ untranslated regions (UTRs) are in lower case and underlined.

FIG. 5 shows the wild-type reference sequences for a segment of the soybean FT1a and JAG1 genes and the corresponding deletions found in the edited ft1a and jag1 alleles found in the SENF2225 soybean variety. The type V Cas nuclease used to generate the deletions cuts 19 bases from the 3′ end of the PAM site (PAM sequence is TTG and the cut site is shown as “/”). The nucleotide residue 20 nucleotides from the PAM site adjacent to the FT1a and JAG1 gRNA protospacers used to generate the deletions is −1 and nucleotide residue 19 nucleotides from that PAM site is +1. The FT1a gRNA protospacer is encoded by SEQ ID NO: 11 and the JAG1 gRNA protospacer is encoded by SEQ ID NO: 17.

FIG. 6 shows the wild-type reference sequences for a segment of the soybean FT1a and JAG1 genes and the corresponding deletions found in the edited ft1a and jag1 alleles found in the SENF2226 soybean variety. The type V Cas nuclease used to generate the deletions cuts 19 bases from the 3′ end of the PAM site (PAM sequence is TTTG and the cut site is shown as “/”). The nucleotide residue 20 nucleotides from the PAM site adjacent to the FT1a and JAG1 gRNA protospacers used to generate the deletions PAM site is −1 and nucleotide residue 19 from the PAM site is +1. The FT1a gRNA protospacer is encoded by SEQ ID NO: 11 and the JAG1 gRNA protospacer is encoded by SEQ ID NO: 17.

FIG. 7 shows the data from BS1/BS2 T0 regenerants with chimeric edited tissues.

FIG. 8 shows harvested seed weigh data from distinct families of T1 plants obtained from the T0 regenerants.

FIG. 9 shows harvested seed count data from distinct families of BS1 and BS2 edited T1 plants obtained from the T0 regenerants. Higher doses of BS1 and BS2 gene edits correlate with reduced seed counts.

FIG. 10 shows seed count per plant, single seed weight, and total seed weight per plant data for the field grown T1 plant −1:5D BS2 homozygotes, NINF1170 checks, and WT checks.

FIG. 11 shows whole plot data for total seed count, single seed weight, and yield for the field grown T1 plant −1:5D BS2 homozygotes, NINF1170 check, and WT check.

FIG. 12 shows whole plot data for late plants per acre for the field grown T1 plant −1:5D BS2 homozygotes, NINF1170 check, and WT check.

FIG. 13 shows the region of the BS1 and BS2 gene which can be targeted for gene edits with a Cas12a guide RNA comprising a spacer RNA encoded by SEQ ID NO: 36. The topmost DNA strand (SEQ ID NO: 39) is a sense strand DNA sequence found in the BS1 gene of SEQ ID NO: 25 and the BS2 gene of SEQ ID NO: 26. The bottom antisense strand (SEQ ID NO: 40) shows the PAM site (solid underlining) and the region corresponding to the SEQ ID NO: 36 spacer RNA coding region. The cleavage site for the Cas12a nuclease is depicted with an arrow.

### DETAILED DESCRIPTION

The phrase “allelic variant” as used herein refers to a polynucleotide or polypeptide sequence variant that occurs in a particular gene at particular locus in a different strain, variety, or isolate of a given organism.

As used herein, the phrase “amorphic allele” refers to an allele of a gene having no gene activity in comparison to the wild-type allele of the gene. Amorphic alleles are also known as null alleles.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the phrase “biological sample” refers to either intact or non-intact (e.g., milled soybean seed or soybean plant tissue, chopped soybean plant tissue, lyophilized tissue) soybean plant tissue. It may also be an extract comprising intact or non-intact seed or soybean plant tissue. The biological sample can comprise flour, meal, syrup, oil, starch, and cereals manufactured in whole or in part to contain soybean plant by-products. In certain embodiments, the biological sample is “non-regenerable” (i.e., incapable of being regenerated into a soybean plant or soybean plant part).

As used herein, the terms “correspond,” “corresponding,” and the like, when used in the context of an nucleotide position, mutation, and/or substitution in any given polynucleotide (e.g., an allelic variant of SEQ ID NO: 3) with respect to the reference polynucleotide sequence (e.g., SEQ ID NO: 3) all refer to the position of the nucleotide in the given sequence that has identity to the nucleotide in the reference nucleotide sequence when the given polynucleotide is aligned to the reference polynucleotide sequence using a pairwise alignment algorithm (e.g., CLUSTAL O 1.2.4 with default parameters).

As used herein, the terms “Cpf1” and “Cas12a” are used interchangeably to refer to the same RNA dependent DNA endonuclease (RdDe).

As used herein, the phrase “elite soybean” refers to soybean plant or part thereof (e.g., seed) which has undergone breeding to provide one or more trait improvements (e.g., desirable agronomic performance (typically commercial production) or superior grain quality). In some cases, an elite line can be an agronomically or otherwise superior line or variety that has resulted from several or many cycles of breeding and selection for one or more trait improvements (e.g., superior agronomic performance or superior grain quality). Similarly, “elite soybean germplasm” is a germplasm resulting from breeding and selection for desirable agronomic performance (typically commercial production). Such germplasm may be agronomically superior germplasm, derived from and/or capable of giving rise to a plant with superior agronomic performance, such as an existing or newly developed elite line of soybean. Elite crop plant lines include plants which are an essentially homozygous, e.g., inbred or doubled haploid. Elite crop plants can include inbred lines used as is or used as pollen donors or pollen recipients in breeding (e.g., used to produce F1 plants). Elite crop plants can include inbred lines which are selfed to produce non-hybrid cultivars or varieties. Elite crop plants can include hybrid F1 progeny of a cross between two distinct elite inbred or doubled haploid plant lines.

As used herein, the phrase “endogenous gene” refers to the native form of a gene unit in its natural location in the genome of an organism.

As used herein, the term “expression” refers to the production of a functional end-product (e.g., an mRNA, guide RNA, or a protein) in either precursor or mature form.

As used herein, the phrase “hypomorphic allele” refers to an allele of a gene with less gene activity than a wild-type allele but more gene activity than an amorphic allele.

As used herein, the terms “include,” “includes,” and “including” are to be construed as at least having the features to which they refer while not excluding any additional unspecified features.

As used herein, the term “isomorphic allele” refers to an allele of a gene having wild-type gene activity.

The term “isolated” as used herein means having been removed from its natural environment.

As used herein, the term “introduced” means providing a nucleic acid (e.g., expression construct) or protein into a cell. Introduced includes reference to the incorporation of a nucleic acid into a eukaryotic or prokaryotic cell where the nucleic acid may be incorporated into the genome of the cell and includes reference to the transient provision of a nucleic acid or protein to the cell. Introduced includes reference to stable or transient transformation methods. Thus, “introduced” in the context of inserting a nucleic acid fragment (e.g., a recombinant DNA construct/expression construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., nuclear chromosome, plasmid, plastid, chloroplast, or mitochondrial DNA), converted into an autonomous replicon, or transiently expressed (e.g., transfected mRNA).

As used herein, a “loss-of-function allele” can include an amorphic allele or a hypomorphic allele of a gene.

As used herein, the phrase “mutated BS1 gene” or “mBS1 gene” refers to an endogenous soybean BS1 gene or allelic variant thereof comprising a loss-of-function allele (e.g., an amorphic allele) of the soybean BS1 gene or an allelic variant thereof. The phrase “mutated BS2 gene” or “mBS2 gene” refers to an endogenous soybean BS2 gene or allelic variant thereof comprising a loss-of-function (e.g., amorphic) allele of the soybean BS2 gene or allelic variant thereof.

As used herein, a “non-natural” or “non-naturally occurring” mutation or allele refers to a mutation or allele in a gene which is generated via human intervention or descended from the mutation generated via human intervention. Non-limiting examples of human intervention which can be used to generate a non-naturally occurring mutation or allele include mutagenesis (e.g., chemical mutagenesis, ionizing radiation mutagenesis), mutagenesis followed by DNA sequence-based screening and selection (TILLING), and targeted genetic modifications (e.g., CRISPR-based methods, TALEN-based methods, zinc finger-based methods).

As used herein, the term “plant” includes reference to an immature or mature whole soybean plant, including a plant from which seed or grain or anthers have been removed. Any seed or embryo that will produce the plant is also considered to be the soybean plant.

As used herein, the term “mutated FT1a gene” or “ft1a gene” refer to an endogenous soybean FT1a gene comprising a loss-of-function allele. The term “ft1a protein” refers to a protein encoded by an endogenous soybean FT1a gene comprising a loss-of-function allele.

As used herein, the term “plant” includes a whole soybean plant and any descendant, cell, tissue, part, or parts of the plant. The term “plant” thus includes reference to an immature or mature whole soybean plant, including a plant from which seed or grain or anthers have been removed.

The term “plant part” include any part(s) of a plant, including, for example and without limitation: seed (including mature seed and immature seed); grain; stover; a plant cutting; a plant cell; a plant cell culture; or a plant organ (e.g., pollen, embryos, pods; flowers, fruits, shoots, leaves, roots, stems, and explants). A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. Regenerable cells in a plant cell or tissue culture may be embryos, protoplasts, meristematic cells, callus, pollen, leaves, anthers, roots, root tips, flowers, or stalks. In contrast, some plant cells are not capable of being regenerated to produce plants and are referred to herein as “non-regenerable” plant cells.

As used herein, the term “variety” refers a group of similar plants that by one or more structural features, genetic features, and/or performance can be distinguished from other varieties within the same species. In certain embodiments, the term variety refers to the botanical taxonomic designation whereby variety is ranked below species or subspecies, as well as the legal definition whereby the term “variety” refers to a commercial plant that is protected under the terms outlined in the International Convention for the Protection of New Varieties of Plants.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

The present disclosure provides for soybean plant cells, plant parts including seed, plants, seed lots, and biological samples comprising a mutated FT1a gene (i.e., comprising a loss-of-function allele of the endogenous FT1a gene), mutated JAG1 gene, mutated BS1 gene, mutated BS2 gene, and/or mutated TFL1b gene. These soybean plants and parts can be utilized for human food, livestock feed, as a raw material in industry, or as breeding material for development of other soybean varieties.

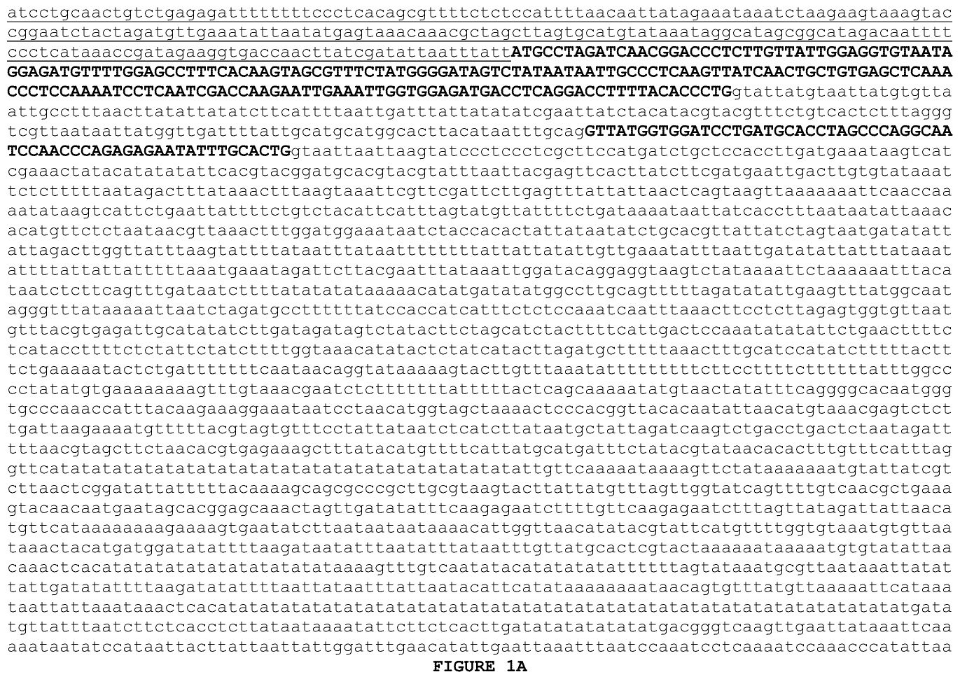

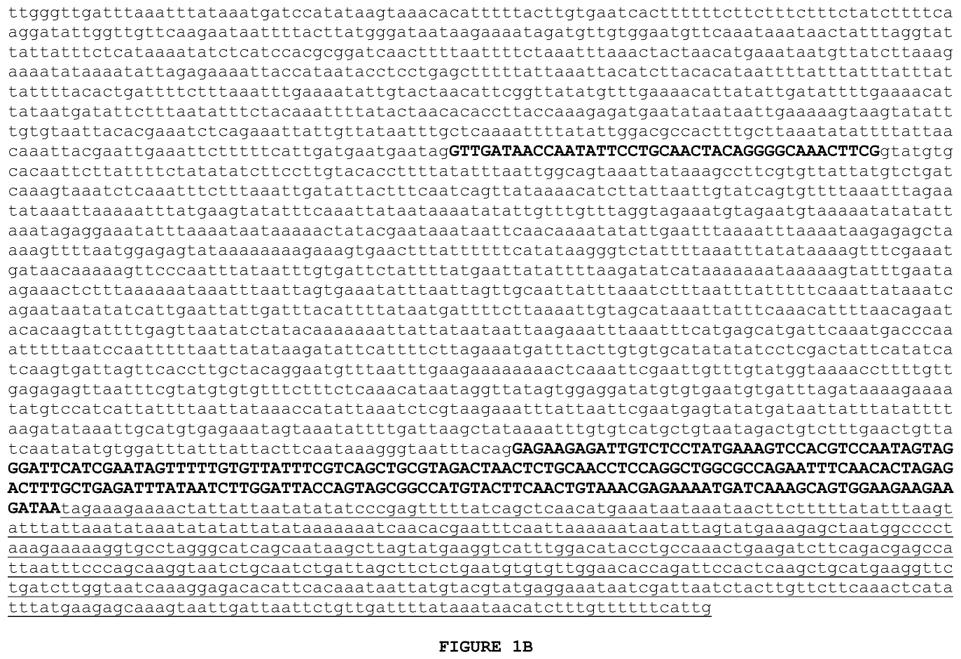

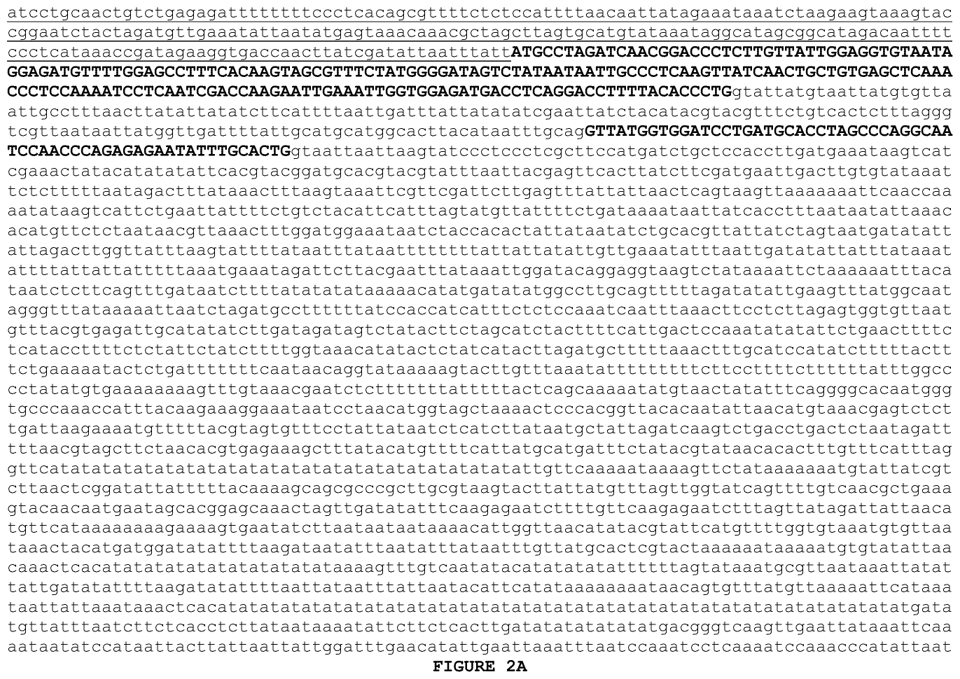

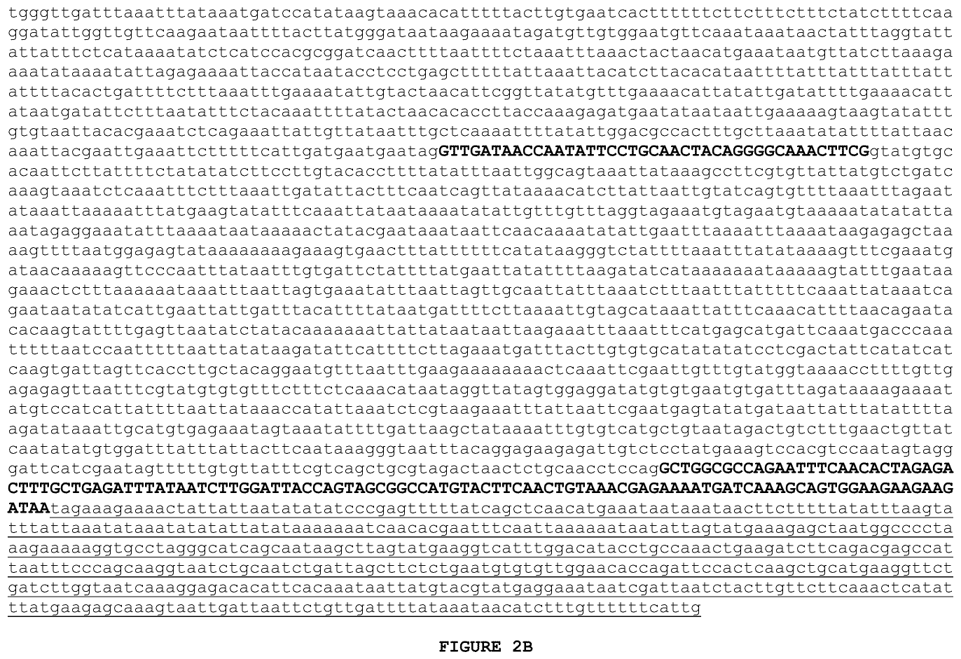

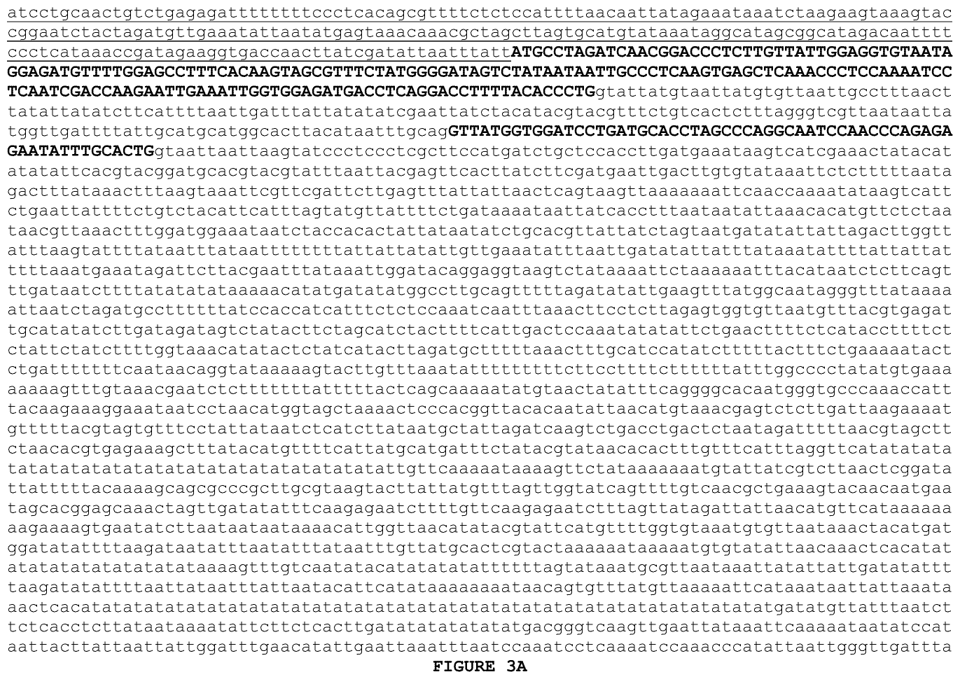

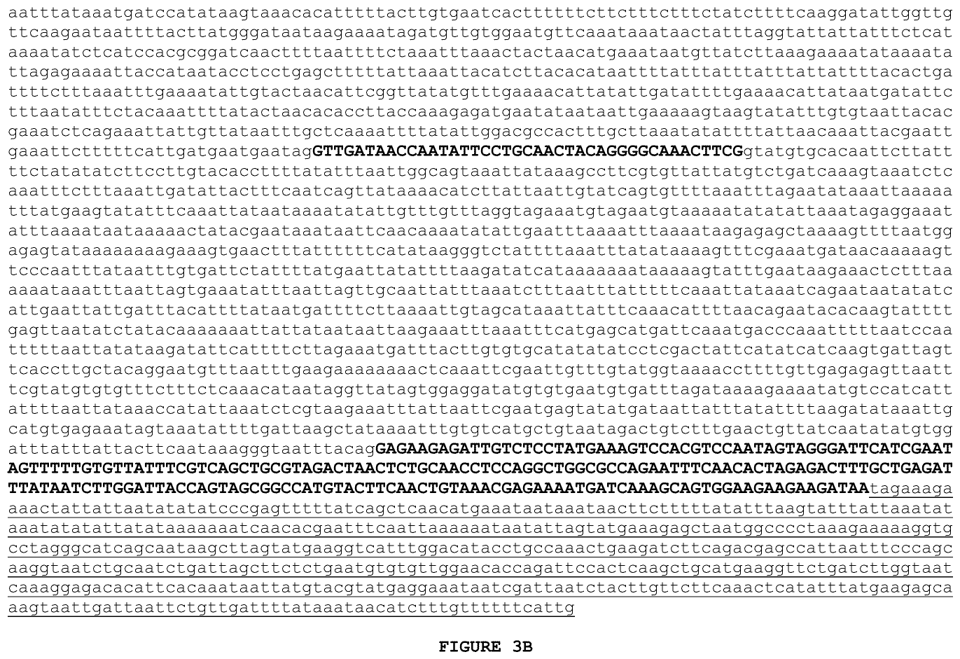

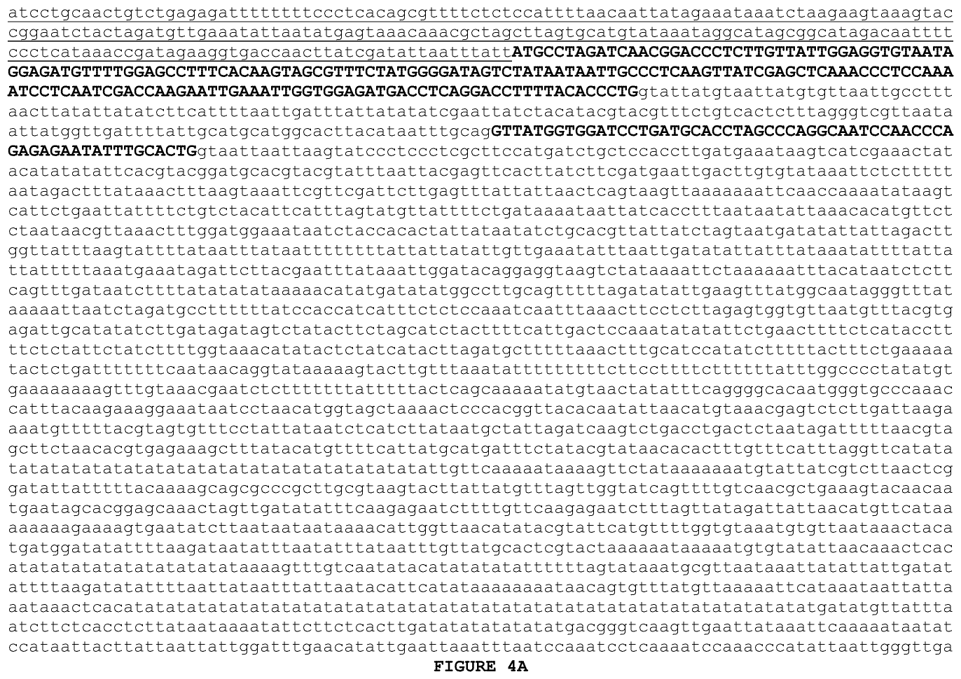

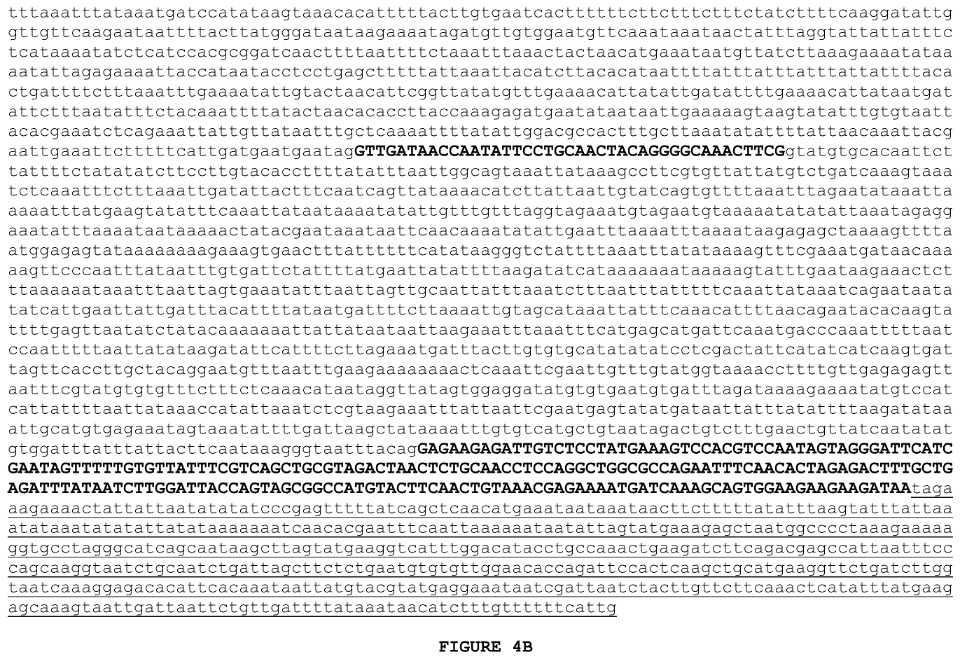

The target endogenous FT1a gene comprises the genomic DNA of SEQ ID NO: 3 and allelic variants thereof located on soybean chromosome 18. The endogenous soybean FT1a gene is located at nucleotides 57,922,912 to 57,928,648 of chromosome 18 of the Glycine max Williams 82genome assembly version 4 (Wm82.a4.v1; Glyma.18G298900 on the world wide web internet site “soybase.org”; Grant et al. Nucl. Acids Res. (2010) 38 (suppl 1): D843-D846. doi: 10.1093/nar/gkp798). Alternative splicing of FT1a gene transcripts result in the mRNA splice variant 1 (Glyma.18G298900.1) as shown in FIG. 1A, B which encodes the FT1a protein of SEQ ID NO: 1 and the mRNA splice variant 2 (Glyma.18G298900.3) shown in FIG. 2A, B which encodes the FT1a protein of SEQ ID NO: 2. Allelic variants of an endogenous soybean FT1a gene include variants which encode FT1a proteins having at least 95%, 96%, 97%, 98%, 99%, 99.5%, 99.6%, 99.7%, 99.8%, or 99.9% sequence identity to SEQ ID NO: 1 or SEQ ID NO 2. Allelic variants of an endogenous soybean FT1a gene also include variants which comprise genomic DNA having at least 95%, 96%, 97%, 98%, 99%, 99.5%, 99.6%, 99.7%, 99.8%, or 99.9% sequence identity to SEQ ID NO: 3. In certain embodiments, allelic variants of the endogenous soybean FT1a gene are isomorphic alleles of the endogenous soybean FT1a gene. The wild-type soybean FT1a gene encodes a member of the phosphatidylethanolamine-binding protein (PEBP) family, which is described by Serre et al. Structure (1998), 6:1255-1265. Residues in the soybean FT1a wild-type protein of SEQ ID NO: 1 that are conserved with other PEBP family members include amino acid residues include residues 65-76, P80, H87, G116, and R119. Residues in the soybean FT1a wild-type protein of SEQ ID NO: 2 that are conserved with other PEBP family members include amino acid residues include residues 65-76, P80, and H87.

Soybean plant cells, plant parts, and plants comprising a loss-of-function allele of the soybean FT1a gene of SEQ ID NO: 3 or an allelic variant thereof are provided. In certain embodiments, the soybean plant cells, plant parts, and plants comprising a loss-of-function allele of the soybean FT1a gene of SEQ ID NO: 3 or an allelic variant thereof can further comprise a loss-of-function allele in any one or more of a JAG1 gene of SEQ ID NO: 18, BS1 gene of SEQ ID NO: 25, BS2 gene of SEQ ID NO: 26, TFL1b gene of SEQ ID NO: 22, and/or an allelic variant thereof. In certain embodiments, one or more of the loss-of-function alleles is a non-natural allele. Examples of loss-of-function alleles can include a deletion, an insertion, and/or a substitution of one or more nucleotides of the endogenous FT1a gene. The insertion, deletion, and/or substitution can be made anywhere in the FT1a gene including, for example, in the promoter region, an exon, an intron, and/or the untranslated regions (5′ UTR or 3′ UTR). In certain embodiments, the loss-of-function allele comprises a deletion, insertion, and/or substitution in the coding region of the FT1a gene. In certain embodiments, a loss-of-function allele of the FT1a gene can comprise a deletion of the entire coding region or any portion of the coding region required for biological activity. In certain embodiments, the loss-of-function allele comprises a deletion, insertion, and/or substitution of one or more nucleotides of exon 1 of mRNA splice variant 1 and 2 (i.e., nucleotides 229 to 429 of SEQ ID NO: 3 or in an equivalent position of an allelic variant of SEQ ID NO: 3), exon 2 of mRNA splice variant 1 and 2 (i.e., nucleotides 596 to 657 of SEQ ID NO: 3 or in an equivalent position of an allelic variant of SEQ ID NO: 3), exon 3 of mRNA splice variant 1 and 2 (i.e., nucleotides 3643 to 3683 of SEQ ID NO: 3 or in an equivalent position of an allelic variant of SEQ ID NO: 3), or in nucleotides corresponding to those of exon 4 of mRNA splice variant 1 (i.e., nucleotides 4999 to 5225 or nucleotides 5104 to 5225 of SEQ ID NO: 3 or in an equivalent position of an allelic variant of SEQ ID NO: 3) of the FT1a gene of SEQ ID NO: 3 or an allelic variant thereof. In certain embodiments, the loss-of-function allele comprises a deletion or substitution of at least 1, 2, 3, 4, 5, 6, 7, 8, 9, 10, 11, 12, 13, 14, 15, 16, 17, 18, 19, 20, 21, 22, 23, 24, 25, or 26 nucleotides of the endogenous soybean FT1a gene of SEQ ID NO: 3 located at nucleotide 331 to 356 of SEQ ID NO: 3 or an allelic variant thereof.

In certain embodiments, the loss-of-function allele comprises a deletion, an insertion, and/or substitution that results in a frameshift mutation and/or a nonsense mutation in the coding region of the FT1a gene. In certain embodiments, loss-of-function alleles of the FT1a gene can comprise a deletion of any number of nucleotides that are not divisible by 3 in an exon of the FT1a gene. In certain embodiments, loss-of-function alleles of the FT1a gene can comprise a deletion of 1, 2, 4, 5, 7, 8, 10, 11, 13, 14, 16, 17, 19, 20, 22, 23, 25, 26, 28, 29, 31, 32, 34, 35, 37, 38, 40, 41, 43, 44, 46, 47, 49, 50, 52, 53, 55, 56, 58, 59, 61, 62, 64, 65, 67, 68, 70, 71, 73, 74, 76, 77, 79, 80, 82, 83, 85, 86, 88, 89, 91, 92, 94, 95, 97, 98, 100, 101, 103, 104, 106, 107, 109, 110, 112, 113, 115, 116, 118, 119, 121, 122, 124, 125, 127, 128, 130, 131, 133, 134, 136, 137, 139, 140, 142, 143, 145, 146, 148, 149, 151, 152, 154, 155, 157, 158, 160, 161, 163, 164, 166, 167, 169, 170, 172, 173, 175, 176, 178, 179, 181, 182, 184, 185, 187, 188, 190, 191, 193, 194, 196, 197, 199, or 200 nucleotides of the endogenous soybean FT1a gene of SEQ ID NO: 3 located at nucleotide 229 to 429 (i.e., the first exon) of SEQ ID NO: 3 and result in a frameshift mutation.

In certain embodiments, the frameshift mutation occurs at nucleotides corresponding to one or more of nucleotides 229 to 429 of SEQ ID NO: 3 or an allelic variant thereof. In certain embodiments, the frameshift mutation occurs at nucleotides corresponding to one or more of nucleotides 340 to 343 of SEQ ID NO: 3 or an allelic variant thereof. In certain embodiments, mutated FT1a genes comprising the loss-of-function allele with a frameshift mutation can comprise the nucleotide sequence of SEQ ID NO: 4 or SEQ ID NO: 5 or an allelic variant thereof. In certain embodiments, such allelic variants of SEQ ID NO: 4 or SEQ ID NO: 5 can comprise a nucleotide sequence having at least 95%, 96%, 97%, 98%, 99%, 99.5%, 99.6%, 99.7%, 99.8%, or 99.9% sequence identity across the entire length of SEQ ID NO: 4 or SEQ ID NO: 5. In certain embodiments, mutated FT1a genes comprising the loss-of-function allele with a frameshift mutation can encode the polypeptide comprising the amino acid sequence of SEQ ID NO: 6 or an allelic variant thereof. In certain embodiments, such allelic variants of SEQ ID NO: 6 can comprise an amino acid sequence having at least 95%, 96%, 97%, 98%, 99%, 99.5%, 99.6%, 99.7%, 99.8%, or 99.9% sequence identity across the entire length of SEQ ID NO: 6. In certain embodiments, the loss-of-function alleles of the FT1a gene can comprise a deletion of an FT1a gene set forth in SEQ ID NO: 13, 21, or an allelic variant thereof comprising the deletion and having at least 95%, 96%, 97%, 98%, 99%, 99.5%, 99.6%, 99.7%, 99.8%, or 99.9% sequence identity across the entire length of SEQ ID NO: 13 or 21. In certain embodiments, loss-of-function alleles of the FT1a gene can comprise a deletion of an FT1a gene set forth in Table 2. In certain embodiments, the deletion in the FT1a gene set forth in Table 2 is a deletion generated by a gRNA comprising a spacer RNA encoded by SEQ ID NO: 11 having the deletion coordinates set forth in column 1 of Table 2. In certain embodiments, loss-of-function alleles of the FT1a gene can comprise a deletion in an FT1a gene corresponding to deleted nucleotides of SEQ ID NO: 3 set forth in column 3 of Table 2. In certain embodiments, loss-of-function alleles of the FT1a gene can comprise a deletion in an allelic variant of SEQ ID NO: 3 having at least 90%, 95%, 97%, 98%, or 99% sequence identity to SEQ ID NO: 3, wherein the deletion corresponds to a deletion of nucleotides in SEQ ID NO: 3 set forth in Table 2. In certain embodiments, the deletion of nucleotides of SEQ ID NO: 3 set forth in column 3 of Table 2 occurs in genomic DNA of SEQ ID NO: 3 or an allelic variant thereof having at least 90%, 95%, 97%, 98%, or 99% sequence identity to SEQ ID NO: 3.

In certain embodiments, the loss-of-function allele comprises an internal deletion that preserves the reading frame of the encoded FT1a proteins while removing at least one, two, or three codons, thus resulting mutant ft1a proteins lacking at least one, two, or three amino acid residues. In certain embodiments, loss-of-function alleles of the FT1a gene can comprise a deletion of any number of nucleotides that are divisible by 3 in an exon of the FT1a gene. In certain embodiments, loss-of-function alleles of the FT1a gene can comprise a deletion of 3, 6, 9, 12, 15, 18, 21, 24, 27, 30, 33, 36, 39, 42, 45, 48, 51, 54, 57, 60, 63, 66, 69, 72, 75, 78, 81, 84, 87, 90, 93, 96, 99, 102, 105, 108, 111, 114, 117, 120, 123, 126, 129, 132, 135, 138, 141, 144, 147, 150, 153, 156, 159, 162, 165, 168, 171, 174, 177, 180, 183, 186, 189, 192, 195, 198, or 201 nucleotides of the endogenous soybean FT1a gene of SEQ ID NO: 3 located at nucleotide 229 to 429 (i.e., within the first exon of mRNA splice forms 1 and 2) of SEQ ID NO: 3 and preserve the reading frame. In certain embodiments, the loss-of-function allele comprises an internal deletion comprising at least nucleotides corresponding to at least nucleotides 343 to 351 of SEQ ID NO: 3 or an allelic variant thereof which preserves the reading frame. In certain embodiments, the loss-of-function allele comprises an internal deletion comprising at least nucleotides encoding amino acids corresponding to N39 to C41 of SEQ ID NO: 1 and SEQ ID NO: 2 or an allelic variant thereof which preserves the reading frame. In these embodiments, the loss-of-function allele can comprise an internal deletion of nucleotides encoding amino acids corresponding to N39 to C41 of SEQ ID NO: 1 and 2 and further comprise deletions of nucleotides encoding P2, R3, S4, T5, D6, P7, L8, V9, I10, G11, G12, V13, I14, G15, D16, V17, L18, E19, P20, F21, T22, S23, S24, V25, S26, M27, G28, I29, V30, Y31, N32, N33, C34, P35, Q36, V37, I38, E42, L43, K44, P45, S46, K47, I48, L49, N50, R51, P52, R53, I54, E55, I56, G57, G58, D59, D60, L61, R62, T63, F64, Y65, T66, and/or L67 of SEQ ID NO: 1, SEQ ID NO: 2, or an allelic variant thereof while preserving the reading frame. In certain embodiments, mutated FT1a genes comprising the loss-of-function allele with an internal deletion can comprise the nucleotide sequence of SEQ ID NO: 7 or SEQ ID NO: 8 or an allelic variant thereof. In certain embodiments, such allelic variants of SEQ ID NO: 7 or SEQ ID NO: 8 can comprise a nucleotide sequence having at least 95%, 96%, 97%, 98%, 99%, 99.5%, 99.6%, 99.7%, 99.8%, or 99.9% sequence identity across the entire length of SEQ ID NO: 7 or SEQ ID NO: 8. In certain embodiments, mutated FT1a genes comprising the loss-of-function allele with an internal deletion can encode the polypeptide comprising the amino acid sequence of SEQ ID NO: 9 and/or SEQ ID NO: 10 or an allelic variant thereof. In certain embodiments, such allelic variants of SEQ ID NO: 9 and/or SEQ ID NO: 10 can comprise an amino acid sequence having at least 95%, 96%, 97%, 98%, 99%, 99.5%, 99.6%, 99.7%, 99.8%, or 99.9% sequence identity across the entire length of SEQ ID NO: 9 and/or SEQ ID NO: 10.

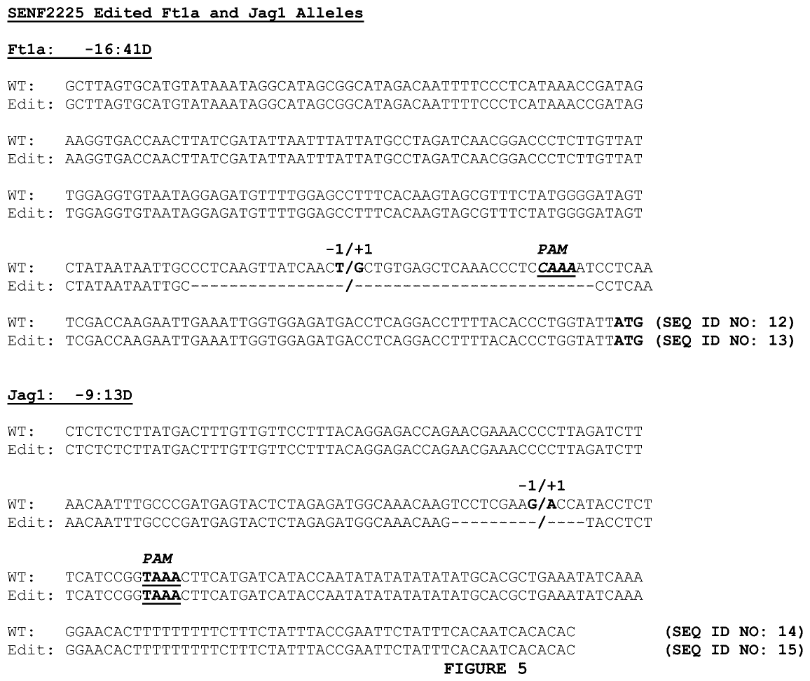

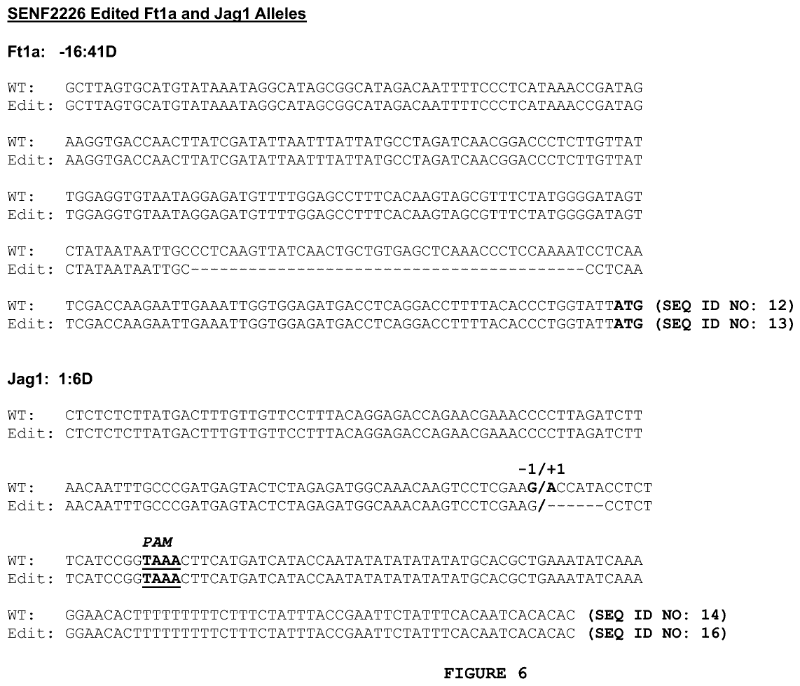

The present disclosure provides for the soybean plant cells, plant parts including seed, plants, and biological samples comprising a mutated JAG1 (mJAG1) gene comprising a loss-of-function mutation of that gene and a loss-of-function mutation in any one or more of an FT1a, BS1, BS2, and/or TFL1b gene. In certain embodiments, one or more of the loss-of-function mutation is a non-natural mutation or allele. These loss-of-function mutations in the mJAG1 gene can be amorphic (null) or hypomorphic alleles of the JAG1 gene, can be homozygous, and include JAG1 mutations disclosed in WO2023/183772 and U.S. Provisional Application 63/269,663, which are each incorporated herein by reference in their entireties. The target endogenous JAG1 gene comprises the genomic DNA of SEQ ID NO: 18 and allelic variants thereof located on soybean chromosome 20. The endogenous soybean JAG1 gene is located at nucleotides 35827671 to 35830107 of chromosome 20 of the Glycine max Wm82.a2.v1 set forth in the https internet site “phytozome-next.jgi.doe.gov/report/transcript/Gmax_Wm82_a2_v1/Glyma.20G116200.1.” Allelic variants of an endogenous soybean JAG1 gene include variants which encode JAG1 proteins having at least 95%, 96%, 98%, 99%, or 99.5% sequence identity to the JAG1 protein encoded by SEQ ID NO: 18. Allelic variants of an endogenous soybean JAG1 gene also include variants which comprise genomic DNA having at least 95%, 96%, 98%, 99%, or 99.5% sequence identity to SEQ ID NO: 18. In certain embodiments, mJAG1 genes can encode the polypeptide encoded by the mutated jag1 gene of SEQ ID NO: 19, SEQ ID NO: 20, or an allelic variant thereof. In certain embodiments, allelic variants of the mJAG1 genes of SEQ ID NO: 19 or 20 can comprise a nucleic acid sequence comprising, consisting essentially of, or consisting of the deletions in the JAG1 gene set forth in Table 3, in SEQ ID NO: 19, in SEQ ID NO: 20, FIG. 5, or in FIG. 6. In certain embodiments, allelic variants of the mJAG1 genes of SEQ ID NO: 19 or 20 can comprise an nucleic acid sequence comprising, consisting essentially of, or consisting of the deletions in the JAG1 gene set forth in Table 3, in SEQ ID NO: 19, in SEQ ID NO: 20, FIG. 5, or in FIG. 6 and having at least 95%, 96%, 98%, 99%, or 99.5% sequence identity across the entire length of SEQ ID NO: 18, 19, 20. In certain embodiments, the mJAG1 gene comprises a deletion corresponding to nucleotides 426 to 430, 424 to 430, or 423 to 430 of the endogenous soybean JAG1 gene of SEQ ID NO: 18 and allelic variants thereof comprising the deletion and having at least at least 95%, 96%, 98%, 99%, or 99.5% sequence identity thereto. In certain embodiments, the mJAG1 gene can comprise a JAG1 gene or an aforementioned allelic variant thereof which comprises a deletion corresponding to nucleotides 426 to 430, 426 to 431, 417 to 429, 424 to 430, or 423 to 430 of the endogenous soybean JAG1 gene of SEQ ID NO: 18. In certain embodiments, the mJAG1 deletion is a null mutation that comprises, consists essentially of, or consists of a deletion corresponding to at least nucleotides 421 to 436, 426 to 431, or 417 to 429 of SEQ ID NO: 18 or an allelic variant thereof comprising, consisting essentially of, or consisting of the deletion and having at least at least 95%, 96%, 98%, 99%, or 99.5% sequence identity thereto. In certain embodiments, the loss-of-function alleles of the JAG1 gene can comprise a deletion of an JAG1 gene set forth in SEQ ID NO: 13, 21, or an allelic variant thereof comprising the deletion and having at least 95%, 96%, 97%, 98%, 99%, 99.5%, 99.6%, 99.7%, 99.8%, or 99.9% sequence identity across the entire length of SEQ ID NO: 13 or 21. In certain embodiments, loss-of-function alleles of the JAG1 gene can comprise a deletion of an JAG1 gene set forth in Table 3. In certain embodiments, the deletion in the FT1a gene set forth in Table 3 is a deletion generated by a gRNA comprising a spacer RNA encoded by SEQ ID NO: 17 having the deletion coordinates set forth in column 1 of Table 3. In certain embodiments, loss-of-function alleles of the JAG1 gene can comprise a deletion in an JAG1 gene corresponding to deleted nucleotides of SEQ ID NO: 18 set forth in column 3 of Table 3. In certain embodiments, loss-of-function alleles of the JAG1 gene can comprise a deletion in an allelic variant of SEQ ID NO: 18 having at least 90%, 95%, 97%, 98%, or 99% sequence identity to SEQ ID NO: 18, wherein the deletion corresponds to a deletion of nucleotides in SEQ ID NO: 18 set forth in Table 3. In certain embodiments, the deletion of nucleotides of SEQ ID NO: 18 set forth in column 3 of Table 3 occurs in genomic DNA of SEQ ID NO: 18 or an allelic variant thereof having at least 90%, 95%, 97%, 98%, or 99% sequence identity to SEQ ID NO: 18.

The present disclosure also provides for the soybean plant cells, plant parts including seed, plants, and biological samples comprising an mBS1 or mBS2 gene. These mBS1 or mBS2 plants and parts can be utilized for human food, livestock feed, as a raw material in industry, or as breeding material for development of other soybean varieties. The target endogenous BS1 comprises the genomic DNA of SEQ ID NO: 25 and allelic variants thereof located on soybean chromosome 10. The endogenous soybean BS1 gene is located at nucleotides 46,684,005 to 46,690,867 of chromosome 10 of the Glyma.Wm82.a1 (Gmax1.01) physical map set forth in the https internet site “spybase.org.” The target endogenous soybean BS2 gene comprises the genomic DNA of SEQ ID NO: 26 and allelic variants thereof located on soybean chromosome 20. The endogenous soybean BS2 gene is located at about nucleotides 37,798,675 to 37,804,788 of chromosome 20 of the Glyma.Wm82.a1 (Gmax1.01) physical map set forth in the https internet site “spybase.org.” The orthologous BS1 gene of the legume Medicago truncatula has been identified as group II member of the TIFY family of transcription regulators (Ge et al. Proc Natl Acad Sci USA. 2016, 113(44):12414-12419, doi: 10.1073/pnas.1611763113; Bai et al. Genomics 2011, 98(2):128-136; Vanholme et al., Trends Plant Sci. 2007, 12(6):239-244). Allelic variants of an endogenous soybean BS1 or BS2 gene include variants which encode BS1 or BS2 proteins having at least 95%, 96%, 98%, 99%, or 99.5% sequence identity to SEQ ID NO: 27 or 28, respectively. Allelic variants of an endogenous soybean BS1 or BS2 gene also include variants which comprise genomic DNA having at least 95%, 96%, 98%, 99%, or 99.5% sequence identity to SEQ ID NO: 25 or SEQ ID NO: 26, respectively. In certain embodiments, amorphic alleles of the BS1 or BS2 gene can comprise a deletion of the entire BS1 or BS2 protein coding sequence or any portion of the BS1 or BS2 protein coding sequence required for biological activity. In certain embodiments, one or more of the loss-of-function or amorphic alleles are non-natural alleles. In certain embodiments, amorphic alleles of the BS1 gene can comprise a deletion in 1, 2, 4, 5, 7, 8, 10, 11, 13, 14, 16, 17, 19, 20, 22, 23, 25, or 26 nucleotides of the endogenous soybean BS1 gene of SEQ ID NO: 25 located at nucleotide 890 to 916 of SEQ ID NO: 25 and result in a frameshift mutation. In certain embodiments, amorphic alleles of the BS1 gene can comprise a deletion in 1, 2, 4, 5, 7, 8, 10, 11, 13, 14, 16, 17, 19, 20, 22, 23, 25, or 26 nucleotides the endogenous soybean BS2 gene of SEQ ID NO: 26 gene located at nucleotide 1572 to 1598 of SEQ ID NO: 26 and result in a frameshift mutation. In certain embodiments, mBS2 genes comprising the amorphic allele with a frameshift mutation can encode the polypeptide comprising the amino acid sequence of SEQ ID NO: 29 or an allelic variant thereof. In certain embodiments, such allelic variants of SEQ ID NO: 29 can comprise an amino acid sequence having at least 95%, 96%, 98%, 99%, or 99.5% sequence identity across the entire length of SEQ ID NO: 29. In certain embodiments, mBS2 genes comprising the amorphic allele with a frameshift mutation can encode the polypeptide comprising the amino acid sequence of SEQ ID NO: 30 or an allelic variant thereof. In certain embodiments, such allelic variants of SEQ ID NO: 30 can comprise an amino acid sequence having at least 95%, 96%, or 98% sequence identity across the entire length of SEQ ID NO: 30.

In certain embodiments, soybean plants homozygous for an mBS1 or mBS2 gene can yield seed lots wherein the number of seeds per kilogram of seeds harvested from the soybean plant homozygous for the mBS1 or mBS2 gene is decreased in comparison to the number of seeds per kilogram harvested from corresponding control soybean plant lacking the mBS1 or mBS2 gene (e.g., a wild-type soybean plant homozygous for a wild-type BS1 and a BS2 gene). In certain embodiments, the number of seeds per kilogram of seeds harvested from the soybean plant homozygous for the mBS1 or mBS2 gene is decreased by up to about 5%, 6%, 7%, 8%, 9%, 10%, 12%, 15%, 18%, 20%, 22%, 24%, 26%, 28%, or 30% in comparison to the number of seeds per kilogram harvested from corresponding control soybean plant lacking the mBS1 or mBS2 gene (e.g., a wild-type soybean plant homozygous for a wild-type BS1 and BS2 gene). In certain embodiments, the number of seeds per kilogram of seeds harvested from the soybean plant homozygous for the mBS1 or mBS2 gene is decreased by about 5% to any one of about 6%, 7%, 8%, 9%, 10%, 12%, 15%, 18%, 20%, 22%, 24%, 26%, 28%, or 30% in comparison to the number of seeds per kilogram harvested from corresponding control soybean plant lacking the mBS1 or mBS2 gene (e.g., a wild-type soybean plant homozygous for a wild-type BS1 and BS2 gene). In certain embodiments, the average weight per 1000 seeds harvested from the soybean plant homozygous for the mBS1 or mBS2 gene is increased by up to about 2%, 4%, 6%, 8%, 9%, 10%, 12% 15%, 18%, 20%, 22%, 24%, 26%, 28%, or 30% in comparison to the weight per 1000 seeds harvested from corresponding control soybean plant lacking the mBS1 or mBS2 gene (e.g., a wild-type soybean plant homozygous for a wild-type BS1 and BS2 gene). In certain embodiments, the weight per 1000 seeds harvested from the soybean plant homozygous for the mBS1 or mBS2 gene is increased by 5% to any of 6%, 8%, 9%, 10%, 12% 15%, 18%, 20%, 22%, 24%, 26%, 28%, or 30% in comparison to the weight per 1000 seeds harvested from corresponding control soybean plant lacking the mBS1 or mBS2 gene (e.g., a wild-type soybean plant homozygous for a wild-type BS1 or BS2 gene). In certain embodiments, the aforementioned mBS1 gene is an amorphic allele of the BS1 gene comprising a deletion in 1, 2, 4, 5, 7, 8, 10, 11, 13, 14, 16, 17, 19, 20, 22, 23, 25, or 26 base pairs in the endogenous soybean BS1 gene of SEQ ID NO: 25 located at nucleotide 890 to 916 of SEQ ID NO: 25 or in the corresponding nucleotides of an allelic variant thereof and result in a frameshift mutation. In certain embodiments, the mBS1 gene comprises a deletion set forth in SEQ ID NO: 32 or the corresponding deletion in an allelic variant of SEQ ID NO: 32. In certain embodiments, the aforementioned mBS2 gene is an amorphic allele of the BS1 gene comprising a deletion in 1, 2, 4, 5, 7, 8, 10, 11, 13, 14, 16, 17, 19, 20, 22, 23, 25, or 26 base pairs in the endogenous soybean BS2 gene of SEQ ID NO: 26 gene located at nucleotide 1572 to 1598 of SEQ ID NO: 26 or in the corresponding nucleotides of an allelic variant thereof and result in a frameshift mutation. In certain embodiments, the mBS2 gene comprises a deletion set forth in SEQ ID NO: 31, 33, 34, or 35 or the corresponding deletion in an allelic variant of SEQ ID NO: 32: 31, 33, 34, or 35. In certain embodiments of any of the aforementioned seed lots, the mBS2 gene encodes the polypeptide comprising the amino acid sequence of SEQ ID NO: 29 or an allelic variant thereof. In certain embodiments, such allelic variants of SEQ ID NO: 29 can comprise an amino acid sequence having at least 95%, 96%, 98%, 99%, or 99.5% sequence identity across the entire length of SEQ ID NO: 29. In certain embodiments of any of the aforementioned seed lots, the mBS2 gene encodes the polypeptide comprising the amino acid sequence of SEQ ID NO: 30 or an allelic variant thereof. In certain embodiments, such allelic variants of SEQ ID NO: 30 can comprise an amino acid sequence having at least 95%, 96%, 98%, 99%, or 99.5% sequence identity across the entire length of SEQ ID NO: 30. In certain embodiments, soybean plants homozygous for an mBS1 gene or an mBS2 gene provided herein can exhibit seed yields which are equivalent to or essentially the same as seed yields of corresponding control soybean plant lacking the mBS1 or mBS2 gene (e.g., a wild-type soybean plant homozygous for a wild-type BS1 and BS2 gene). In certain embodiments, the mBS1 or mBS2 gene is generated with a gRNA comprising a spacer region encoded by SEQ ID NO: 36. In certain embodiments, the content of one or more amino acids is increased in the seeds harvested from the soybean plant homozygous for the mBS1 or mBS2 gene in comparison to the content of those amino acids in seeds harvested from corresponding control soybean plant lacking the mBS1 or mBS2 gene (e.g., a wild-type soybean plant homozygous for a wild-type BS1 or BS2 gene). In certain embodiments, the content of one or more amino acids selected from the group consisting of aspartate, threonine, serine, glutamate, proline, glycine, alanine, valine, isoleucine, leucine, phenylalanine, histidine, lysine, and arginine is increased in the seeds harvested from the soybean plant homozygous for the mBS1 or mBS2 gene in comparison to the content of those amino acids in seeds harvested from corresponding control soybean plant lacking the mBS1 or mBS2 gene (e.g., a wild-type soybean plant homozygous for a wild-type BS1 or BS2 gene).

In certain embodiments, soybean plants comprising a loss-of-function allele in one or more of an endogenous soybean FT1a, JAG1, BS1, and/or BS2 gene can further comprise a loss-of-function (e.g., amorphic) allele in the soybean TFL1b gene which is an ortholog of the Arabidopsis Terminal Flower 1 gene. The soybean TFL1b ortholog is found in the https internet database “soybase.org” as Glyma.19G194300 and also known as the soybean stem growth habit gene Dtl (Liu et al. Plant Physiol. 2010 May; 153(1):198-210. doi: 10.1104/pp. 109.150607. Epub 2010 Mar. 10. PMID: 20219831; PMCID: PMC2862436). The TFL1b wild-type genomic DNA is provided as SEQ ID NO: 22, the TFL1b wild-type coding sequence is provided as SEQ ID NO: 23, and the TFL1b protein is provided as SEQ ID NO: 24. Loss-of-function mutations in the TFL1b gene can be obtained by gene editing techniques (e.g., by use of CRISPR/Cas9, CRISPR/Cas12, TALEN, or aZFN-mediated site-specific mutagenesis of the TFL1b gene). In certain embodiments, the loss-of-function mutations in the TFL1b gene are non-natural. In certain embodiments, the hypomorphic mutations in the TFL1b gene include those disclosed in US Patent Application publication US20230235350A1, which is incorporated herein by reference in its entirety.

In certain embodiments, the yield of the soybean plant comprising a loss-of-function allele of the endogenous soybean FT1a, JAG1, BS1, BS2, and/or TFL1b gene is increased in comparison to the yield of a wild-type control soybean plant lacking the loss-of-function allele. Increased yield of the soybean plant can be measured in a number of ways, including pod count per plant, seed count per plant, total harvested seed weight per plant, or total harvested seed weight per unit area (e.g., seed weight per acre or seed weight per hectare). In certain embodiments, the increased yield can result from an improved response to stress, including an abiotic stress (e.g., drought, heat, cold, and/or salt stress).

In certain embodiments, the pod count per soybean plant comprising the loss-of-function allele in the FT1a gene and optionally a loss-of-function mutation in a JAG1, BS1, BS2, and/or TFL1b gene is increased in comparison to the pod count per plant for a wild-type control soybean plant lacking the loss-of-function allele. In certain embodiments, the pod count per plant is increased by at least about 2%, 3%, 4%, 5%, 6%, 7%, 8%, 9%, 10%, 12%, 15%, 18%, 20%, 25%, 30%, 35%, 40%, 45%, or 50% in comparison to the pod count per plant from the corresponding wild-type control soybean plant lacking the loss-of-function allele. In certain embodiments, the seed count per plant comprising the loss-of-function allele in the FT1a gene is increased in comparison to the seed count per plant for a wild-type control soybean plant lacking the loss-of-function allele. In certain embodiments, the seed count per plant comprising the loss-of-function allele in the FT1a gene is increased by at least about 2%, 3%, 4%, 5%, 6%, 7%, 8%, 9%, 10%, 12%, 15%, 18%, 20%, 25%, 30%, 35%, 40%, 45%, or 50% in comparison to the seed count per plant from the corresponding wild-type control soybean plant lacking the loss-of-function allele. In certain embodiments, the total harvested seed weight per plant comprising the loss-of-function allele in the FT1a gene is increased in comparison to the total harvested seed weight per plant for a wild-type control soybean plant lacking the loss-of-function allele. In certain embodiments, the total harvested seed weight per plant is increased by at least about 2%, 3%, 4%, 5%, 6%, 7%, 8%, 9%, 10%, 12%, 15%, 18%, 20%, 25%, 30%, 35%, 40%, 45%, or 50% in comparison to the total harvested seed weight per plant from the corresponding wild-type control soybean plant lacking the loss-of-function allele. In certain embodiments, the total harvested seed weight per unit area for soybean plants comprising the loss-of-function allele in the FT1a gene is increased in comparison to the total harvested seed weight per unit area for a wild-type control soybean plant lacking the loss-of-function allele. In certain embodiments, the total harvested seed weight per unit area for soybean plants comprising the loss-of-function allele in the FT1a gene is increased by at least about 2%, 3%, 4%, 5%, 6%, 7%, 8%, 9%, 10%, 12%, 15%, 18%, 20%, 25%, 30%, 35%, 40%, 45%, or 50% in comparison to the total harvested seed weight per unit area from the corresponding wild-type control soybean plant lacking the loss-of-function allele. In certain embodiments, the average weight of 1000 seeds obtained from the soybean plant is equivalent to or essentially the same as the average weight of 1000 seeds obtained from a wild-type control soybean plant lacking the loss-of-function allele.

In certain embodiments, the pod count per plant, seed count per plant, total harvested seed weight per plant, and/or total harvested seed weight per unit area are increased when the soybean plant comprising the loss-of-function allele in the FT1a gene and optionally a loss-of-function mutation in a JAG1, BS1, BS2, and/or TFL1b gene is grown under stress in comparison to pod count per plant, seed count per plant, total harvested seed weight per plant, and/or total harvested seed weight per unit area for a wild-type control soybean plant lacking the loss-of-function allele grown under stress. Non-limiting examples of stresses include drought, cold, heat, salt, shade, nutrient deficiency, high planting density, and the presence of pests or pathogens. In certain embodiments, the stress comprises an abiotic stress. In certain embodiments, the abiotic stress comprises drought, cold, heat, salt, stress. In these embodiments, the pod count per plant, seed count per plant, total harvested seed weight per plant, and/or total harvested seed weight per unit area can be increased when the plant comprising the loss-of-function allele in the FT1a gene is grown under drought stress in comparison to pod count per plant, seed count per plant, total harvested seed weight per plant, and/or total harvested seed weight per unit area for a wild-type control soybean plant lacking the loss-of-function allele grown under drought stress.

In certain embodiments, the pod count per plant, seed count per plant, total harvested seed weight per plant, and/or total harvested seed weight per unit area are increased in comparison to a control (e.g., check) when the soybean plant comprising the loss-of-function allele in the FT1a gene and optionally a loss-of-function mutation in a JAG1, BS1, BS2, and/or TFL1b gene is grown for a full growing season. In certain embodiments, the seed plant comprising the loss-of-function allele in the FT1a gene are planted at or after (e.g., on or within a week) of the earliest initial planting date provided by the USDA Risk Management Agency for the maturity zone where they are planted. Non-limiting examples of Risk Management Agency (RMA) replant crop insurance dates can range from about April 1 in the southeastern United States to about May 5 in northern sections of the midwestern United States (see the https internet site “soybeanresearchinfo.com/wp-content/uploads/2022/01/2700-003-23_Planting-Date-V1.pdf”). In certain embodiments, at least 50%, 70%, 80%, or 90% of plants in the soybean crop comprising the loss-of-function allele in the FT1a gene have 95% of their pods at full maturity color when harvested. Full maturity color is variety-dependent and can be gray, tan, or brown. In certain embodiments, the soybean crop comprising the loss-of-function allele in the FT1a gene is a full season variety for the soybean maturity group zone where it is grown and wherein the seed are harvested at or after a full growing season for the full season variety. Soybeans comprising the loss-of-function allele in the FT1a gene can fall into any of the 13 maturity group designations ranging from 000, 00, 0, or 1 to X. Maturity groups can also be represented by Arabic numbers and tenths (e.g., “5.8”). Soybean maturity groups 00 to VIII are typically grown in the U.S. (See https internet site “soybeanresearchinfo.com/research-highlight/delineating-optimal-soybean-maturity-groups-across-the-united-states/”).

Soybean seed lots comprising the soybean seeds comprising the loss-of-function allele in the FT1a, JAG1, BS1, BS2, and/or TFL1b gene(s) are provided. In certain embodiments, soybean plants comprising the mutated FT1a gene can yield seed lots wherein the average weight of 1000 seeds in the seed lot is equivalent to or essentially the same as the average weight of 1000 seeds in a control seed lot obtained from a wild-type control plant lacking the loss-of-function allele in the FT1a gene (e.g., a wild-type soybean plant homozygous for a wild-type FT1a gene). In certain embodiments, the average number of seeds per kilogram of seeds in the seed lot is equivalent to or essentially the same as the average number of seeds per kilogram of seeds in a control seed lot obtained from a wild-type control soybean plant lacking the loss-of-function allele in the FT1a gene. In certain embodiments, the seed lot is packaged in lots comprising about 50 to 60 pounds (i.e., about 22.7 to 27.2 kilograms) of seeds.

Also provided are polynucleotides comprising any of the aforementioned mutated FT1a, JAG1, BS1, BS2, and/or TFL1b genes or fragments thereof. In certain embodiments, polynucleotides comprising the sequence of SEQ ID NO: 4, SEQ ID NO 5, SEQ ID NO 7, or SEQ ID NO 8 or allelic variants thereof are provided. In certain embodiments, the allelic variants of SEQ ID NO: 4, SEQ ID NO 5, SEQ ID NO 7, or SEQ ID NO 8 will comprise sequences having at least 95%, 96, 97%, 98%, 99%, 99.5%, 99.6%, 99.7%, 99.8%, or 99.9% sequence identity across the entire length of SEQ ID NO: 4, SEQ ID NO 5, SEQ ID NO 7, or SEQ ID NO 8 with the proviso that the sequences are not identical to across their entire length to SEQ ID NO: 3. In certain embodiments, the polynucleotides encode a polypeptide comprising the amino acid sequence of SEQ ID NO: 6, SEQ ID NO 9, or SEQ ID NO 10 or an allelic variant thereof. In certain embodiments, the encoded allelic variant will comprise a polypeptide having at least 95%, 96%, 97%, 98%, 99%, 99.5%, 99.6%, 99.7%, 99.8%, or 99.9% sequence identity across the entire length of SEQ ID NO: 6, SEQ ID NO 9, or SEQ ID NO 10 with the proviso that the sequences are not identical to SEQ ID NO: 1 or SEQ ID NO 2. In certain embodiments, the polynucleotide is an isolated polynucleotide.

Biological samples and soybean by-products comprising any of the aforementioned or otherwise disclosed polynucleotides including those set forth in the sequence listing, Table 2, and/or Table 3 are also provided. In certain embodiments, the by-products are processed products are made from the soybean plants of the disclosure or their seeds, including: (a) soybean seed meal (defatted or non-defatted); (b) extracted soybean proteins, oils, sugars, syrups, and starches; (c) soy fermentation products; (d) soybean based animal feed or human food products (e.g., feed and food comprising soybean seed meal (defatted or non-defatted) and other ingredients (e.g., other cereal grains, other seed meal, other protein meal, other oil, other starch, other sugar, a binder, a preservative, a humectant, a vitamin, and/or mineral); (e) a pharmaceutical; (f) raw or processed biomass (e.g., cellulosic and/or lignocellulosic material; silage); and (g) various industrial products.

Methods of using the soybean plants, seeds, and seed lots of the disclosure to produce soybean by-products are also provided. Such methods will typically include at least one processing step of cleaning, cracking, flaking, crushing, macerating, pressing, extracting, expelling, and/or extruding the seed.

This disclosure is also directed to methods for producing a soybean plant having a loss-of-function allele of the endogenous soybean FT1a, JAG1, BS1, BS2, and/or TFL1b gene by crossing a first parent soybean plant with a second parent soybean plant wherein the first or second parent soybean plant comprises the loss-of-function allele. Further, both the first and second parent soybean plants can comprise the loss-of-function allele. Any such methods using a soybean plant comprising the loss-of-function allele are part of this disclosure: selfing, backcrosses, hybrid production, crosses to populations, and the like. All plants produced using a soybean plant comprising the loss-of-function allele as a parent are within the scope of this disclosure, including plants derived from a soybean plant having the loss-of-function allele. Also provided are the F₁ progeny soybean plants produced from the crossing of a soybean plant comprising the loss-of-function allele with any other soybean plant, F₁ seed, and various parts of the F₁ soybean plant. The following describes breeding methods that can be used with soybean plants of the disclosure in the development of further soybean plants. One such embodiment is a method for developing a progeny soybean plant in a soybean plant breeding program comprising: obtaining the soybean plant, or its parts, comprising a loss-of-function allele of the endogenous soybean FT1a gene and utilizing said plant or plant parts as a source of breeding material; and selecting a progeny plant having the loss-of-function allele. Breeding steps that can be used in the soybean plant breeding program include pedigree breeding, backcrossing, mutation breeding, and recurrent selection. In conjunction with these steps, techniques such as restriction fragment polymorphism enhanced selection, genetic marker enhanced selection (for example SNP or SSR markers), and the making of double haploids can be utilized.

Field crops are bred through techniques that take advantage of the plant's method of pollination. A soybean plant of the disclosure can be self-pollinated, sib-pollinated, or cross pollinated to create a pedigree soybean plant. A plant is self-pollinated if pollen from one flower is transferred to the same or another flower of the same plant. A plant is sib-pollinated when individuals within the same family or variety are used for pollination. A plant is cross-pollinated if the pollen comes from a flower on a different plant from a different family or variety. The terms “cross-pollination” and “out-cross” as used herein do not include self-pollination or sib-pollination. Soybean plants (Glycine max) are recognized to be naturally self-pollinated plants which, while capable of undergoing cross-pollination, rarely do so in nature. Insects are reported by some researchers to carry pollen from one soybean plant to another and it generally is estimated that less than one percent of soybean seed formed in an open planting can be traced to cross-pollination, i.e., less than one percent of soybean seed formed in an open planting is capable of producing F₁ hybrid soybean plants.

Any other suitable breeding, selection, or growing methods may be used. Choice of the particular breeding or selection method will vary depending on environmental factors, population size, and the like.

In certain embodiments, soybean plant cells, plant parts (e.g., seeds), and plants comprising at least one mutation in the endogenous soybean FT1a, JAG1, BS1, BS2, and/or TFL1b gene and a transgenic locus are provided. In certain embodiment, the at least one mutation in the endogenous soybean FT1a, JAG1, BS1, BS2, and/or TFL1b is combined with one or more soybean GM events providing tolerance to any one or a combination of glyphosate-based, glufosinate-based, HPPD inhibitor-based, sulfonylurea- or imidazolinone-based, AHAS- or ALS-inhibiting and/or auxin-type (e.g., dicamba, 2,4-D) herbicides and/or an insect resistance trait. GM events that can be combined with the mutations disclosed herein include Event EE-GM3 (aka FG-072, MST-FGØ72-3, described in WO2011063411, USDA-APHIS Petition 09-328-01p), Event SYHTOH2 (aka 0H2, SYN-ØØØH2-5, described in WO2012/082548 and 12-215-01p), Event DAS-68416-4 (aka Enlist Soybean, described in WO2011/066384 and WO2011/066360, USDA-APHIS Petition 09-349-Olp), Event DAS-44406-6 (aka Enlist E3, DAS-444Ø6-6, described in WO2012/075426 and USDA-APHIS 11-234-01p), Event MON87708 (dicamba-tolerant event of Roundup Ready 2 Xtend Soybeans, described in WO2011/034704 and USDA-APHIS Petition 10-188-01p, MON-87708-9), Event MON89788 (aka Genuity Roundup Ready 2 Yield, described in WO2006/130436 and USDA-APHIS Petition 06-178-01p), Event 40-3-2 (aka Roundup Ready, GTS 40-3-2, MON-Ø4Ø32-6, described in USDA-APHIS Petition 93-258-01), Event A2704-12 (aka LL27, ACS-GMØØ5-3, described in WO2006108674 and USDA-APHIS Petition 96-068-01p), Event 127 (aka BPS-CV127-9, described in WO2010/080829), Event A5547-127 (aka LL55, ACS-GMØØ6-4, described in WO2006108675 and in USDA-APHIS Petition 96-068-01p), event MON87705 (MON-877Ø5-6, Vistive Gold, published PCT patent application WO2010/037016, USDA-APHIS Petition 09-201-01p), or event DP305423 (aka DP-3Ø5423-1, published PCT patent application WO2008/054747, USDA-APHIS Petition 06-354-01p), or EE-GM5 is combined with a combination of the following events: Event MON98788×MON87708 (aka Roundup Ready 2 Xtend Soybeans, MON-877Ø8-9×MON-89788-1), Event HOS×Event 40-3-2 (aka Plenish High Oleic Soybeansx Roundup Ready Soybeans), Event EE-GM3×EE-GM2 (aka FG-072×LL55, described in WO2011063413), Event MON 87701×MON 89788 (aka Intacta RR2 Pro Soybean, MON-877Ø1-2×MON-89788-1), DAS-81419-2×DAS-44406-6 (aka Conkesta™ Enlist E3™ Soybean, DAS-81419-2×DAS-444Ø6-6), Event DAS-68416-4×Event MON 89788 (aka Enlist™ RoundUp Ready® 2 Soybean, DAS-68416-4×MON-89788-1), Event MON-87769-7×Event MON-89788-1 (aka Omega-3×Genuity Roundup Ready 2 Yield Soybeans), Event MON 87705×Event MON 89788 (aka Vistive Gold, MON-877Ø5-6×MON-89788-1), or Event MON87769×Event MON89788 (aka Omega-3×Genuity Roundup Ready 2 Yield Soybeans, MON-87769-7×MON-89788-1), where all published PCT patent applications or US national stages thereof are incorporated herein by reference in there entireties. Also provided herein are soybean plant cells, plant parts (e.g., seeds), and plants comprising at least one mutation in the endogenous soybean FT1a, JAG1, BS1, BS2, and/or TFL1b and a modification of any of the aforementioned transgenic events or transgenic events. Modifications of the transgenic events include those disclosed in: WO2022/026375, WO2022/026379, WO2022/026390, WO2022/026395, WO2022/026403; US Patent Applic. Pub. Nos. US20220030822 and US20230250441; and U.S. Pat. No. 11,242,534, which are each incorporated herein by reference in their entireties.

Methods of producing a soybean seed lot comprising: (i) growing a population of soybean plants comprising a mutated FT1a, JAG1, BS1, BS2, and/or TFL1b gene to maturity; and (ii) harvesting seed from the population of soybean plants of step (i) at maturity, thereby producing the soybean seed lot, wherein the soybean plants are homozygous for the mutated FT1a gene. In certain embodiments, the seed lot is packaged in lots comprising about 50 to 60 pounds (i.e., about 22.7 to 27.2 kilograms).

Also provided herein are methods of treating the soybean seeds and seed lots of the disclosure and the resultant treated seeds and seed lots. Seeds can be treated with such fertilizers, biological agents, nematicides, insecticides, and fungicides by methods including in-furrow applications or by coating (e.g., with a drum coater, rotary coater, tumbling drum, fluidized bed, and/or spouted bed apparatus). Methods and compositions including various binders, fillers, film coats, and active ingredients such as fertilizers, surfactants, plant growth regulators, crop desiccants, fungicides, bacteriocides, bacteriostats, insecticides, and insect repellants for coating seeds that can be adapted for use with seeds provided herein are disclosed in U.S. Pat. No. 10,745,578, which is incorporated herein by reference in its entirety.

The disclosure also provides a method of making a soybean plant comprising a mutated FT1a, JAG1, BS1, BS2, and/or TFL1b gene. In certain embodiments, the methods can comprise making a deletion, an insertion and/or a substitution which results in a mutated FT1a, JAG1, BS1, BS2, and/or TFL1b gene. Gene editing molecules of use in methods provided herein include molecules capable of introducing a double-strand break (“DSB”) or single-strand break (“SSB”) at a specific site or sequence in a double-stranded DNA, such as in genomic DNA or in a target gene located within the genomic DNA as well as accompanying guide RNA. In certain embodiments, the loss-of-function allele results from introduction of a DSB at a target site in the FT1a gene (e.g., SEQ ID NO: 3 or an allelic variant thereof), JAG1 gene, BS1 gene, BS2 gene, and/or the TFL1b gene to induce non-homologous end joining (NHEJ) at the site of the break followed by recovery of desired loss-of-function alleles. In certain embodiments, the loss-of-function allele results from introduction of a DSB at a target site in the FT1a gene (e.g., SEQ ID NO: 3 or an allelic variant thereof) followed by homology-directed repair (HDR), microhomology-mediated end joining (MMEJ), or NHEJ to introduce a desired donor or other DNA template polynucleotide at the DSB, followed by recovery of the desired loss-of-function allele. Examples of such gene editing molecules include: (a) a nuclease comprising an RNA-guided nuclease, an RNA-guided DNA endonuclease or RNA directed DNA endonuclease (RdDe), a class 1 CRISPR type nuclease system, a type II Cas nuclease, a Cas9, a nCas9 nickase, a type V Cas nuclease, a Cas12a nuclease, a nCas12a nickase, a Cas12d (CasY), a Cas12e (CasX), a Cas12b (C2c1), a Cas12c (C2c3), a Cas12i, a Cas12j, a Cas14, an engineered nuclease, a codon-optimized nuclease, a zinc-finger nuclease (ZFN) or nickase, a transcription activator-like effector nuclease (TAL-effector nuclease or TALEN) or nickase (TALE-nickase), an Argonaute, and a meganuclease or engineered meganuclease; (b) a polynucleotide encoding one or more nucleases capable of effectuating site-specific alteration (including introduction of a DSB or SSB) of a target nucleotide sequence; (c) a guide RNA (gRNA) for use with an RNA-guided nuclease, or a DNA encoding a gRNA for use with an RNA-guided nuclease; (d) optionally donor DNA template polynucleotides suitable for insertion at a break in genomic DNA by homology-directed repair (HDR) or microhomology-mediated end joining (MMEJ); and (e) optionally other DNA templates (e.g., dsDNA, ssDNA, or combinations thereof) suitable for insertion at a break in genomic DNA (e.g., by non-homologous end joining (NHEJ).

In certain embodiments, the mutated FT1a, JAG1. BS1, BS2, and/or TFL1b gene and plant cells, parts including seeds, and plants comprising the mutated FT1a. BS1. BS2, and/or TFL1b gene are generated by CRISPR technology. CRISPR technology for editing the genes of eukaryotes is disclosed in US Patent Application Publications 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 endonuclease and corresponding guide RNAs and PAM sites are disclosed in US Patent Application Publication 2016/0208243 A1. Plant RNA promoters for expressing CRISPR guide RNA and plant codon-optimized CRISPR Cas9 endonuclease are disclosed in International Patent Application PCT/US2015/018104 (published as WO 2015/131101 and claiming priority to U.S. Provisional Patent Application 61/945,700). Methods of using CRISPR technology for genome editing in plants are disclosed in US Patent Application Publications US 2015/0082478A1 and US 2015/0059010A1 and in International Patent Application PCT/US2015/038767 A1 (published as WO 2016/007347 and claiming priority to U.S. Provisional Patent Application 62/023,246). All of the patent publications referenced in this paragraph are incorporated herein by reference in their entirety. In certain embodiments, an RNA-guided endonuclease that leaves a blunt end following cleavage of the target site is used. Blunt-end cutting RNA-guided endonucleases include Cas9, Cas12c, Cas12i, and Cas 12h (Yan et al., 2019). In certain embodiments, an RNA-guided endonuclease that leaves a staggered single stranded DNA overhanging end following cleavage of the target site following cleavage of the target site is used. Staggered-end cutting RNA-guided endonucleases include Cas12a, Cas12b, and Cas12e.

Guide RNA molecules comprising a spacer RNA molecule which targets the FT1a gene of SEQ ID NO: 3 or an allelic variant thereof are provided. In certain embodiments, the spacer RNA molecule targets a portion of exon 1 (i.e., nucleotides 229 to 429 of SEQ ID NO: 3 or in an equivalent position of an allelic variant of SEQ ID NO: 3), exon 2 (i.e., nucleotides 596 to 657 of SEQ ID NO: 3 or in an equivalent position of an allelic variant of SEQ ID NO: 3), exon 3 (i.e., nucleotides 3643 to 3683 of SEQ ID NO: 3 or in an equivalent position of an allelic variant of SEQ ID NO: 3), or nucleotides corresponding to exon 4 (i.e., nucleotides 4999 to 5225 or nucleotides 5104 to 5225 of SEQ ID NO: 3 or in an equivalent position of an allelic variant of SEQ ID NO: 3) of the FT1a gene of SEQ ID NO: 3 or an allelic variant thereof. In certain embodiments, the spacer RNA molecule comprises the RNA encoded by SEQ ID NO: 11. Guide RNAs comprising a spacer RNA molecule encoded by SEQ ID NO: 11 can be used in conjunction with a Cas12a nuclease to generate mutated FT1a genes which: (i) comprise a deletion in the endogenous FT1a gene of SEQ ID NO: 3 or an allelic variant thereof; (ii) comprise deletions of 1, 2, 3, 4, 5, 6, 7, 8, 9, 10, 11, 12, 13, 14, 15, 16, 17, 18, 19, 20, 21, 22, 23, 24, 25, or 26 nucleotides of the endogenous soybean FT1a gene of SEQ ID NO: 3 located at nucleotide 331 to 356 of SEQ ID NO: 3 or in an equivalent position of an allelic variant of SEQ ID NO: 3; (iii) comprise the sequence of SEQ ID NO: 4, SEQ ID NO: 5, SEQ ID NO: 7, or SEQ ID NO: 8 or allelic variants thereof; or SEQ ID NO: 4 or SEQ ID NO: 5, SEQ ID NO: 7, or SEQ ID NO: 8; or (iv) encode the polypeptides of SEQ ID NO: 6, SEQ ID NO: 9, or SEQ ID NO: 10 or allelic variants thereof. Guide RNAs which target the JAG1 gene include type V Cas nuclease guide RNAs comprising the spacer sequence encoded by SEQ ID NO: 17 and guide RNAs disclosed in WO2023/183772 and U.S. Provisional Application 63/269,663, which are each incorporated herein by reference in their entireties. Guide RNAs which target the BS1 and/or BS2 gene include V Cas nuclease guide RNAs comprising the spacer sequence encoded by SEQ ID NO: 36 and disclosed in U.S. Patent Application Ser. No. 63/490,375, which is incorporated herein by reference in its entirety. Guide RNAs which target the TFL1b gene include those disclosed in US Patent Application publication US20230235350A1, which is incorporated herein by reference in its entirety.

CRISPR-type genome editing can be adapted for use in the plant cells and methods provided herein in several ways. CRISPR elements, e.g., gene editing molecules comprising CRISPR endonucleases and CRISPR guide RNAs including single guide RNAs or guide RNAs in combination with tracrRNAs or scoutRNA, or polynucleotides encoding the same, are useful in effectuating genome editing without remnants of the CRISPR elements or selective genetic markers occurring in progeny. In certain embodiments, the CRISPR elements are provided directly to the eukaryotic cell (e.g., soybean plant cells), systems, methods, and compositions as isolated molecules, as isolated or semi-purified products of a cell free synthetic process (e.g., in vitro translation), or as isolated or semi-purified products of in a cell-based synthetic process (e.g., such as in a bacterial or other cell lysate). In certain embodiments, soybean plants or soybean plant cells used in the systems, methods, and compositions provided herein can comprise a transgene that expresses a CRISPR endonuclease (e.g., a Cas9, a Cpf1-type or other CRISPR endonuclease). In certain embodiments, one or more CRISPR endonucleases with unique PAM recognition sites can be used. Guide RNAs (sgRNAs or crRNAs and a tracrRNA) to form an RNA-guided endonuclease/guide RNA complex which can specifically bind sequences in the gDNA target site that are adjacent to a protospacer adjacent motif (PAM) sequence. The type of RNA-guided endonuclease typically informs the location of suitable PAM sites and design of crRNAs or sgRNAs. G-rich PAM sites, e.g., 5′-NGG are typically targeted for design of crRNAs or sgRNAs used with Cas9 proteins. Examples of PAM sequences include 5′-NGG (Streptococcus pyogenes), 5′-NNAGAA (Streptococcus thermophilus CRISPR1), 5′-NGGNG (Streptococcus thermophilus CRISPR3), 5′-NNGRRT or 5′-NNGRR (Staphylococcus aureus Cas9, SaCas9), and 5′-NNNGATT (Neisseria meningitidis). T-rich PAM sites (e.g., 5′-TTN or 5′-TTTV, where “V” is A, C, or G) are typically targeted for design of crRNAs or sgRNAs used with Cas12a proteins. In some instances, Cas12a can also recognize a 5′-CTA PAM motif. Other examples of potential Cas12a PAM sequences include TTN, CTN, TCN, CCN, TTTN, TCTN, TTCN, CTTN, ATTN, TCCN, TTGN, GTTN, CCCN, CCTN, TTAN, TCGN, CTCN, ACTN, GCTN, TCAN, GCCN, and CCGN (wherein N is defined as any nucleotide). Cpf1 endonuclease and corresponding guide RNAs and PAM sites are disclosed in US Patent Application Publication 2016/0208243 A1, which is incorporated herein by reference for its disclosure of DNA encoding Cpf1 endonucleases and guide RNAs and PAM sites.

In certain embodiments, the mutated FT1a gene and plant cells, parts including seeds, and plants comprising the mutated FT1a gene are generated by use of zinc finger nucleases or zinc finger nickases. Zinc-finger nucleases are site-specific endonucleases comprising two protein domains: a DNA-binding domain, comprising a plurality of individual zinc finger repeats that each recognize between 9 and 18 base pairs, and a DNA-cleavage domain that comprises a nuclease domain (typically Fok1). The cleavage domain dimerizes in order to cleave DNA; therefore, a pair of ZFNs are required to target non-palindromic target polynucleotides. In certain embodiments, zinc finger nuclease and zinc finger nickase design methods which have been described (Urnov et al. (2010) Nature Rev. Genet., 11:636-646; Mohanta et al. (2017) Genes vol. 8,12: 399; Ramirez et al. Nucleic Acids Res. (2012); 40(12): 5560-5568; Liu et al. (2013) Nature Communications, 4: 2565) can be adapted for use in the methods set forth herein. The zinc finger binding domains of the zinc finger nuclease or nickase provide specificity and can be engineered to specifically recognize any desired target DNA sequence. The zinc finger DNA binding domains are derived from the DNA-binding domain of a large class of eukaryotic transcription factors called zinc finger proteins (ZFPs). The DNA-binding domain of ZFPs typically contains a tandem array of at least three zinc “fingers” each recognizing a specific triplet of DNA. A number of strategies can be used to design the binding specificity of the zinc finger binding domain. One approach, termed “modular assembly”, relies on the functional autonomy of individual zinc fingers with DNA. In this approach, a given sequence is targeted by identifying zinc fingers for each component triplet in the sequence and linking them into a multifinger peptide. Several alternative strategies for designing zinc finger DNA binding domains have also been developed. These methods are designed to accommodate the ability of zinc fingers to contact neighboring fingers as well as nucleotide bases outside their target triplet. Typically, the engineered zinc finger DNA binding domain has a novel binding specificity, compared to a naturally occurring zinc finger protein. Engineering methods include, for example, rational design and various types of selection. Rational design includes, for example, the use of databases of triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, e.g., U.S. Pat. Nos. 6,453,242 and 6,534,261, both incorporated herein by reference in their entirety. Exemplary selection methods (e.g., phage display and yeast two-hybrid systems) can be adapted for use in the methods described herein. In addition, enhancement of binding specificity for zinc finger binding domains has been described in U.S. Pat. No. 6,794,136, incorporated herein by reference in its entirety. In addition, individual zinc finger domains may be linked together using any suitable linker sequences. Examples of linker sequences are publicly known, e.g., see U.S. Pat. Nos. 6,479,626; 6,903,185; and 7,153,949, incorporated herein by reference in their entirety. The nucleic acid cleavage domain is non-specific and is typically a restriction endonuclease, such as Fok1. This endonuclease must dimerize to cleave DNA. Thus, cleavage by Fok1 as part of a ZFN requires two adjacent and independent binding events, which must occur in both the correct orientation and with appropriate spacing to permit dimer formation. The requirement for two DNA binding events enables more specific targeting of long and potentially unique recognition sites. Fok1 variants with enhanced activities have been described and can be adapted for use in the methods described herein; see, e.g., Guo et al. (2010) J. Mol. Biol., 400:96-107.

In certain embodiments, the mutated FT1a gene and plant cells, parts including seeds, and plants comprising the mutated FT1a gene are generated by use of TAL-effector nucleases or TALENs. Transcription activator like effectors (TALEs) are proteins secreted by certain Xanthomonas species to modulate gene expression in host plants and to facilitate the colonization by and survival of the bacterium. TALEs act as transcription factors and modulate expression of resistance genes in the plants. Recent studies of TALEs have revealed the code linking the repetitive region of TALEs with their target DNA-binding sites. TALEs comprise a highly conserved and repetitive region consisting of tandem repeats of mostly 33 or 34 amino acid segments. The repeat monomers differ from each other mainly at amino acid positions 12 and 13. A strong correlation between unique pairs of amino acids at positions 12 and 13 and the corresponding nucleotide in the TALE-binding site has been found. The simple relationship between amino acid sequence and DNA recognition of the TALE binding domain allows for the design of DNA binding domains of any desired specificity. TALEs can be linked to a non-specific DNA cleavage domain to prepare genome editing proteins, referred to as TAL-effector nucleases or TALENs. As in the case of ZFNs, a restriction endonuclease, such as Fok1, can be conveniently used. Methods for use of TALENs in plants have been described and can be adapted for use in the methods described herein, see Mahfouz et al. (2011) Proc. Natl. Acad. Sci. USA, 108:2623-2628; Mahfouz (2011) GM Crops, 2:99-103; and Mohanta et al. (2017) Genes vol. 8,12: 399). TALE nickases have also been described and can be adapted for use in methods described herein (Wu et al.; Biochem Biophys Res Commun. (2014); 446(1):261-6; Luo et al; Scientific Reports 6, Article number: 20657 (2016)).

Various treatments can be used for delivery of gene editing molecules and/or other molecules to a plant cell. In certain embodiments, one or more treatments is employed to deliver the gene editing or other molecules (e.g., comprising a polynucleotide, polypeptide or combination thereof) into a plant cell, e.g., through barriers such as a cell wall, a plasma membrane, a nuclear envelope, and/or other lipid bilayer. In certain embodiments, a polynucleotide-, polypeptide-, or RNP (ribonucleoprotein)-containing composition comprising the molecules are delivered directly, for example by direct contact of the composition with a plant cell. Aforementioned compositions can be provided in the form of a liquid, a solution, a suspension, an emulsion, a reverse emulsion, a colloid, a dispersion, a gel, liposomes, micelles, an injectable material, an aerosol, a solid, a powder, a particulate, a nanoparticle, or a combination thereof can be applied directly to a plant, plant part, plant cell, or plant explant (e.g., through abrasion or puncture or otherwise disruption of the cell wall or cell membrane, by spraying or dipping or soaking or otherwise directly contacting, by microinjection). For example, a plant cell or plant protoplast is soaked in a liquid genome editing molecule-containing composition. In certain embodiments, the composition is delivered using negative or positive pressure, for example, using vacuum infiltration or application of hydrodynamic or fluid pressure. In certain embodiments, the composition is introduced into a plant cell or plant protoplast, e.g., by microinjection or by disruption or deformation of the cell wall or cell membrane, for example by physical treatments such as by application of negative or positive pressure, shear forces, or treatment with a chemical or physical delivery agent such as surfactants, liposomes, or nanoparticles; see, e.g., delivery of materials to cells employing microfluidic flow through a cell-deforming constriction as described in US Published Patent Application 2014/0287509, incorporated by reference in its entirety herein. Other techniques useful for delivering the composition to a eukaryotic cell, plant cell or plant protoplast include: ultrasound or sonication; vibration, friction, shear stress, vortexing, cavitation; centrifugation or application of mechanical force; mechanical cell wall or cell membrane deformation or breakage; enzymatic cell wall or cell membrane breakage or permeabilization; abrasion or mechanical scarification (e.g., abrasion with carborundum or other particulate abrasive or scarification with a file or sandpaper) or chemical scarification (e.g., treatment with an acid or caustic agent); and electroporation. In certain embodiments, the composition is provided by bacterially mediated (e.g., Agrobacterium sp., Rhizobium sp., Sinorhizobium sp., Mesorhizobium sp., Bradyrhizobium sp., Azotobacter sp., Phyllobacterium sp.) transfection of the plant cell or plant protoplast with a polynucleotide encoding the genome editing molecules (e.g., RNA dependent DNA endonuclease, RNA dependent DNA binding protein, RNA dependent nickase, ABE, or CBE, and/or guide RNA); see, e.g., Broothaerts et al. (2005) Nature, 433:629-633). Any of these techniques or a combination thereof are alternatively employed on a plant explant, plant part or tissue or intact plant (or seed) from which a plant cell is optionally subsequently obtained or isolated; in certain embodiments, the composition is delivered in a separate step after the plant cell has been isolated.

In certain embodiments, the methods for generating the soybean plant cell, soybean plant parts, or soybean plants comprise: (i) screening a population of soybean plant cells, parts, or plants for the presence of a loss-of-function allele in the endogenous soybean FT1a gene of SEQ ID NO: 3 or an allelic variant thereof; and (ii) isolating a soybean plant cell, soybean plant part, or soybean plant comprising a loss-of-function allele of the soybean FT1a gene of SEQ ID NO: 3 or an allelic variant thereof.

In certain embodiments, the population of soybean plant cells, parts, or plants which are screened for the presence of a loss-of-function allele in the endogenous soybean FT1a gene of SEQ ID NO: 3 are first pre-screened by screening of phenotypic characteristics plants having loss-of-function mutations in an FT1a gene of SEQ ID NO: 3 or an allelic variant thereof. In certain embodiments, such phenotypic characteristics include increased pod count per plant; seed count per plant; and/or total harvested seed weight per plant in comparison to pod count per plant; seed count per plant; and/or total harvested seed weight per plant for a wild-type control soybean plant lacking the loss-of-function allele. In certain embodiments, such phenotypic characteristics include increased pod count per plant; seed count per plant; and/or total harvested seed weight per plant in comparison to pod count per plant; seed count per plant; and/or total harvested seed weight per plant for a wild-type control soybean plant lacking the loss-of-function allele where the screened and control plants are grown under stress conditions (e.g., abiotic stress including drought, cold, heat, or salt stress). In certain embodiments, plants exhibiting one or more of the aforementioned phenotypic characteristics are then subjected to screening for the presence of a loss-of-function allele in the endogenous soybean FT1a gene of SEQ ID NO: 3 and soybean plants comprising loss-of-function alleles in the endogenous soybean FT1a gene of SEQ ID NO: 3 are identified and/or selected.

In certain embodiments, the population of soybean plant cells, parts, or plants which are screened for the presence of a loss-of-function allele in the endogenous soybean FT1a gene of SEQ ID NO: 3, JAG1 gene of SEQ ID NO: 18, BS1 gene of SEQ ID NO: 25, BS2 gene of SEQ ID NO: 26, TFL1B gene of SEQ ID NO: 22, and/or an allelic variant thereof have been subjected to one or more mutagenesis treatments. Loss-of-function alleles of the endogenous soybean FT1a gene can be generated by mutagenesis methods known in the art, such as chemical mutagenesis or radiation mutagenesis. Suitable chemical mutagens include ethyl methanesulfonate (EMS), sodium azide, methylnitrosourea (MNU), and diepoxybutane (DEB). Suitable radiation includes x-rays, fast neutron radiation, and gamma radiation.

Soybean plant cells, parts, or plants comprising a loss-of-function allele of the endogenous FT1a, JAG1, BS1, BS2, and/or TFL1b gene can be generated using mutagenesis and identified by TILLING (Targeting Induced Local Lesions IN Genomes) or identified using EcoTILLING. TILLING is a general reverse genetics technique that uses mutagenesis methods to create libraries of mutagenized individuals that are later subjected to high throughput screens for the discovery of mutations. In addition to allowing efficient detection of induced mutations, high-throughput TILLING technology is ideal for the detection of natural mutations. EcoTILLING is a method that uses TILLING techniques to look for natural mutations in individuals (Barkley and Wang. Current genomics vol. 9,4 (2008): 212-26. doi:10.2174/138920208784533656). Identified mutations can then be introduced into desirable genetic backgrounds by crossing the mutant with a plant of the desired genetic background and performing a suitable number of backcrosses to cross out the originally undesired parent background. A more detailed description of methods and compositions for TILLING are disclosed in US Patent Application Publication 2004/0053236 A1, which is incorporated herein by reference in its entirety and can be adapted for use in the methods provided herein for identifying soybean plant cells, parts, or plants comprising a loss-of-function allele of the endogenous FT1a gene.

In certain embodiments, the screening comprises analyzing pod count per plant, seed count per plant, total harvested seed weight per plant, and/or total harvested seed weight per unit area in one or more candidate plants or one or more candidate plant populations. In these embodiments, an increase in pod count per plant, seed count per plant, total harvested seed weight per plant, and/or total harvested seed weight per unit area in comparison to a wild-type control soybean plant lacking the loss-of-function allele is indicative of a soybean plant cell, soybean plant part, or soybean plant comprising the loss-of-function allele. In certain embodiments, the screening is conducted on a population of plants grown under stress. Suitable examples of stress conditions include drought, salt, cold, heat, salt, shade, nutrient deficiency, high planting density, and the presence of pests or pathogens.

Methods for determining whether a soybean plant cell, plant part, or plant comprises a loss-of-function allele of the endogenous soybean FT1a gene are provided. Methods for determining the presence or absence of the loss-of-function allele can be used in, for example, breeding programs for identification, selection, introgression, and the like.

In certain embodiments, the methods comprise analyzing a polynucleotide comprising a portion of SEQ ID NO: 3 or an allelic variant thereof or analyzing an RNA encoded by a portion of SEQ ID NO: 3 or an allelic variant thereof from the plant cell, plant part, or plant. In certain embodiments, an insertion, deletion, and/or substitution of one or more nucleotides in the polynucleotide or RNA is indicative of the presence of the loss-of-function allele. Detection of the loss-of-function allele in a nucleic acid sample (e.g., DNA, RNA, or cDNA) can be achieved by any combination of nucleic acid amplification (e.g., PCR amplification), hybridization, sequencing, and/or mass-spectrometry based techniques. In certain embodiments, such detection is achieved by amplification and/or hybridization-based detection methods using a primer (e.g., selective amplification primers) and/or probe (e.g., capable of selective hybridization or generation of a specific primer extension product) which specifically recognizes the FT1a gene (e.g., a portion of SEQ ID NO: 3 or an allelic variant thereof). Such primers and/or probes can comprise or consist of about 15, 20, 25, 30, 40, 45 or 50 more contiguous nucleotides of SEQ ID NO: 3 or an allelic variant thereof. In certain embodiments, the primers or probes can comprise or consist of about 10 to 50 contiguous nucleotides, about 10 to 40 contiguous nucleotides, about 10 to 30 contiguous nucleotides or about 15 to 30 contiguous nucleotides of SEQ ID NO: 3 or an allelic variant thereof. In certain embodiments, the hybridization probes (e.g., polynucleotides comprising at least about 15 to 30 base pairs of SEQ ID NO: 3 or an allelic variant thereof) can comprise detectable labels (e.g., fluorescent, radioactive, epitope, and chemiluminescent labels). In certain embodiments, the FT1a gene can be directly sequenced using nucleic acid sequencing technologies, including whole genome sequencing.

In certain embodiments, the methods comprise analyzing a polypeptide encoded by SEQ ID NO: 3, a portion thereof, or an allelic variant thereof from the soybean plant cell, plant part, or plant. In certain embodiments, an insertion, deletion, and/or substitution of one or more amino acid residues of the polypeptide or a change in the biologic or biochemical activity of the polypeptide is indicative of the presence of the loss-of-function allele. Detection of the loss-of-function allele based on the polypeptide can be determined by methods well known in the art such as activity assays, western blots using antibodies capable of specifically binding the polypeptide, enzyme-linked immunosorbent assays (ELISA), radioimmunoassays (RIA), immunohistochemistry, immunocytochemistry, immunofluorescence, and the like.

In certain optional embodiments, the soybean plant cells disclosed herein are non-regenerable soybean plant cells. In certain optional embodiments provided herein, the soybean plant cells, soybean plant propagules (e.g., a seed, seedling, ovule, embryo, pollen, root, stem, leaf, shoot, explant, or callus), and soybean plants provided herein are not produced by an exclusively biological process. In certain optional embodiments provided herein, the methods for producing soybean plant cells, soybean plant propagules (e.g., a seed, seedling, ovule, embryo, pollen, root, stem, leaf, shoot, explant, or callus), and soybean plants provided herein are not exclusively biological processes.

The following non-limiting numbered embodiments also form part of the present disclosure:
1. A soybean plant cell comprising: (i) a loss-of-function allele of the endogenous soybean FT1a gene of SEQ ID NO: 3 or an allelic variant thereof; (ii) a loss-of-function allele of an endogenous soybean JAG1 gene of SEQ ID NO: 18 or an allelic variant thereof; (iii) a loss-of-function allele of an endogenous soybean BS1 gene of SEQ ID NO: 25 or an allelic variant thereof; and/or (iv) a loss-of-function allele of an endogenous soybean BS2 gene of SEQ ID NO: 26 or an allelic variant thereof.
2. The soybean plant cell of embodiment 1, wherein the plant cell is homozygous for the loss-of-function allele of the soybean FT1a gene of SEQ ID NO: 3 or allelic variant thereof.
3. The soybean plant cell of embodiment 1 or embodiment 2, wherein the loss-of-function allele comprises an amorphic allele of the FT1a gene of SEQ ID NO: 3 or allelic variant thereof, optionally wherein the amorphic allele is a non-natural allele.
4. The soybean plant cell of any one of embodiments 1-3, wherein the loss-of-function allele comprises a hypomorphic allele of the FT1a gene, optionally wherein the hypomorphic allele is a non-natural allele.
5. The soybean plant cell of any one of embodiments 1-4, wherein the loss-of-function allele comprises a frameshift mutation or a nonsense mutation in the coding region of the FT1a gene of SEQ ID NO: 3 or allelic variant thereof.
6. The soybean plant cell of any one of embodiments 1-5, wherein the loss-of-function allele comprises a frameshift mutation or a nonsense mutation in the first exon of the FT1a gene of SEQ ID NO: 3 or allelic variant thereof; optionally wherein the frameshift or nonsense mutation occurs at nucleotides corresponding to nucleotides 340 to 343 of SEQ ID NO: 3 or an allelic variant thereof.
7. The soybean plant cell of any one of embodiments 1-6, wherein the loss-of-function allele comprises the frameshift mutation of SEQ ID NO: 4 or SEQ ID NO: 5 or wherein the ft1a gene comprising the frameshift mutation encodes the protein of SEQ ID NO: 6.
8. The soybean plant cell of any one of embodiments 1-7, wherein;
(a) the loss-of-function allele of the FT1a gene comprises an internal deletion comprising: (i) at least nucleotides corresponding to nucleotides 318 to 345, 319 to 356, 320 to 352, 321 to 348, 322 to 350, 323 to 328, 324 to 328, 325 to 347, 326 to 349, 327 to 347, 328 to 348, 329 to 347, 330 to 348, 331 to 347, 332 to 347, 333 to 347, 334 to 345, 335 to 347, 336 to 347, 337 to 347, 338 to 345, 339 to 345, 340, 341 to 345, 342 to 344, 343 to 347, 343 to 351, 344 to 345, 345 to 347, 346 to 347, 347 to 350, 348 to 351, or 331 to 371 of SEQ ID NO: 3 or an allelic variant thereof; (ii) at least nucleotides encoding amino acids corresponding to N39 to C41 of SEQ ID NO: 1 or SEQ ID NO: 2; optionally in either (i) or (ii) wherein the internal deletion preserves the reading frame of the encoded ft1a mutant protein comprising the loss-of-function allele with respect to amino acid residues of the ft1a mutant protein which have not been deleted; (iii) at least nucleotides corresponding to a deletion of an FT1a gene set forth in Table 2; or (iv) at least nucleotides corresponding to a deletion of an FT1a gene set forth in SEQ ID NO: 13, 21, or an allelic variant thereof;
(b) the loss-of-function allele of the JAG1 gene comprises an internal deletion comprising: (i) at least nucleotides corresponding to a deletion of a JAG1 gene set forth in Table 3; (ii) at least nucleotides corresponding to a deletion of a JAG1 gene set forth in SEQ ID NO: 15, 16, 19, 20, or an allelic variant thereof; or (iii) at least nucleotides corresponding to nucleotides 408 to 426, 409 to 427, 410 to 429, 411 to 428, 412 to 428, 413 to 427, 414 to 426, 415 to 427, 416 to 427, 417 to 426, 418 to 426, 419 to 426, 420 to 426, 421 to 426, 422 to 426, 423 to 426, 424 to 427, 425 to 427, 426 to 429, 427 to 428, 429 to 430, 430, or 431 of SEQ ID NO: 18 or an allelic variant thereof;
(c) the loss-of-function alleles of the BS1 gene comprises an internal deletion comprising at least nucleotides corresponding to a deletion in a BS1 gene set forth in SEQ ID NO: 32 or an allelic variant thereof; and/or
(d) the loss-of-function alleles of the BS2 gene comprises an internal deletion comprising at least nucleotides corresponding to a deletion in a BS2 gene set forth in SEQ ID NO: 31, 33, 34, 35, or an allelic variant thereof; optionally wherein the loss-of-function alleles in (a), (b), (c), and/or (d) are non-natural alleles.
9. The soybean plant cell of embodiment 8, wherein the internal deletion comprises: (i) the internal deletion of SEQ ID NO: 7 or SEQ ID NO: 8; or (ii) the internal deletion which encodes a protein corresponding to the protein of SEQ ID NO: 9 and/or SEQ ID NO: 10.
10. The soybean plant cell of any one of embodiments 1-9, with the proviso that the soybean plant cell is not exclusively produced by an essentially biological method.
11. The soybean plant cell of any one of embodiments 1-10, wherein the soybean plant cell comprises elite soybean germplasm.
12. The soybean plant cell of any one of embodiments 1-11, wherein the soybean plant cell further comprises: (i) one or more mutations in a distinct soybean gene, optionally wherein the distinct soybean gene is an endogenous soybean TFL1b gene (SEQ ID NO: 22) or an allelic variant thereof and the mutation is a loss-of-function mutation; and/or (ii) one or more transgenes, optionally wherein said transgenes encode proteins or RNAs conferring herbicide tolerance or pest tolerance.
13. A soybean plant part comprising the soybean plant cell of any one of embodiments 1-12, optionally wherein the soybean plant part is not produced by an exclusively biological process.
14. The soybean plant part of embodiment 13, wherein the part is a stem, root, leaf, flower, pod, or seed.
15. The soybean plant part of embodiment 13 or embodiment 14, wherein the part is a seed.
16. The soybean plant part of any one of embodiments 13-15, wherein the part is a seed and wherein the total harvested seed weight per acre or hectare of a population of soybean plants grown from the seed is increased in comparison to total harvested seed weight per acre or hectare of a population of wild-type control soybean plants lacking the loss-of-function allele.
17. The soybean plant part of any one of embodiment 13-16, wherein the part is a seed and wherein the average weight of the seed is equivalent to the average weight of a wild-type control seed lacking the loss-of-function allele.
18. The soybean plant part of any one of embodiment 13-17, wherein the part is a pod; optionally wherein pod count for a soybean plant which is homozygous for the loss-of-function mutation and from which the pod was obtained is increased in comparison to pod count of a wild-type control plant lacking the loss-of-function allele.
19. A soybean seed lot comprising the seed of any one of embodiment 14-17, optionally wherein the soybean seed lot is not produced by an exclusively biological process.
20. The soybean seed lot of embodiment 19, wherein the average weight of 1000 seeds in the seed lot is equivalent to the average weight of 1000 seeds in a control seed lot obtained from a wild-type control plant lacking the loss-of-function allele.
21. The soybean seed lot of embodiment 19 or embodiment 20, wherein the average number of seeds per kilogram of seeds in the seed lot is equivalent to the average number of seeds per kilogram of seeds in a control seed lot obtained from a wild-type control soybean plant lacking the loss-of-function allele.
22. A soybean plant comprising the soybean plant cell of any one of embodiments 1-12, optionally wherein the soybean plant is not produced by an exclusively biological process.
23. The soybean plant of embodiment 22, wherein pod count per plant; seed count per plant; and/or total harvested seed weight per plant are increased in comparison to pod count per plant; seed count per plant; and/or total harvested seed weight per plant for a wild-type control soybean plant lacking the loss-of-function allele.
24. The soybean plant of embodiment 22 or embodiment 23, wherein pod count per plant; seed count per plant; and/or total harvested seed weight per plant are increased when the plant is grown under stress in comparison to pod count per plant; seed count per plant; and/or total harvested seed weight per plant for a wild-type control soybean plant lacking the loss-of-function allele grown under stress, optionally wherein the stress comprises abiotic stress.
25. The soybean plant of any one of embodiments 22-24, wherein the average weight of 1000 seeds obtained from the soybean plant is equivalent to the average weight of 1000 seeds obtained from a wild-type control soybean plant lacking the loss-of-function allele.
26. A biological sample comprising a nucleic acid containing a loss-of-function allele of the soybean FT1a gene of SEQ ID NO: 3 or an allelic variant thereof, a loss-of-function allele of the soybean JAG1 gene of SEQ ID NO: 18 or an allelic variant thereof, a loss-of-function allele of an endogenous soybean BS1 gene of SEQ ID NO: 25 or an allelic variant thereof, a loss-of-function allele of an endogenous soybean TFL1b gene of SEQ ID NO: 22 or an allelic variant thereof, and/or a loss-of-function allele of an endogenous soybean BS2 gene of SEQ ID NO: 26 or an allelic variant thereof.
27. The biological sample of embodiment 26, wherein the sample comprises seed meal or a tissue sample homogenate, optionally wherein the tissue sample comprises a sample of leaf, flower, pod, seed, stem, or root tissue.
28. The biological sample of embodiment 26 or embodiment 27, wherein the loss-of-function allele comprises a frameshift mutation or a nonsense mutation in the coding region of the FT1a gene of SEQ ID NO: 3 or allelic variant thereof.
29. The biological sample of any one of embodiments 26-28, wherein the loss-of-function allele comprises a frameshift mutation or a nonsense mutation in the first exon of the FT1a gene of SEQ ID NO: 3 or allelic variant thereof; optionally wherein the frameshift or nonsense mutation occurs at nucleotides corresponding to nucleotides 340 to 343 of SEQ ID NO: 3 or an allelic variant thereof, optionally wherein the frameshift mutation comprises the frameshift mutation of SEQ ID NO: 4 or SEQ ID NO: 5 or wherein the ft1a gene comprising the frameshift mutation encodes the protein of SEQ ID NO: 6.
30. The biological sample of any one of embodiments 26-29, wherein the loss-of-function allele comprises an internal deletion comprising: (i) at least nucleotides corresponding to nucleotides 318 to 345, 319 to 356, 320 to 352, 321 to 348, 322 to 350, 323 to 328, 324 to 328, 325 to 347, 326 to 349, 327 to 347, 328 to 348, 329 to 347, 330 to 348, 331 to 347, 332 to 347, 333 to 347, 334 to 345, 335 to 347, 336 to 347, 337 to 347, 338 to 345, 339 to 345, 340, 341 to 345, 342 to 344, 343 to 347, 343 to 351, 344 to 345, 345 to 347, 346 to 347, 347 to 350, 348 to 351, or 331 to 371 in the FT1a gene of SEQ ID NO: 3 or an allelic variant thereof; (ii) at least nucleotides encoding amino acids corresponding to N39 to C41 of SEQ ID NO: 1 or SEQ ID NO: 2; optionally in either (i) or (ii) wherein the internal deletion preserves the reading frame of the encoded ft1a mutant protein comprising the loss-of-function allele with respect to amino acid residues of the ft1a mutant protein which have not been deleted; (iii) at least nucleotides corresponding to a deletion of an FT1a gene set forth in Table 2; (iv) at least nucleotides corresponding to nucleotides 408 to 426, 409 to 427, 410 to 429, 411 to 428, 412 to 428, 413 to 427, 414 to 426, 415 to 427,416 to 427,417 to 426, 418 to 426, 419 to 426,420 to 426,421 to 426, 422 to 426, 423 to 426, 424 to 427, 425 to 427, 426 to 429, 427 to 428, 429 to 430, 430, or 431 in the JAG1 gene of SEQ ID NO: 18 or an allelic variant thereof; (v): at least nucleotides corresponding to a deletion of a JAG1 gene set forth in Table 3; and/or (vi) at least nucleotides corresponding to a deletion of a JAG1 gene set forth in SEQ ID NO: 15, 16, 19, 20, or an allelic variant thereof.
31. The biological sample of embodiment 30, wherein the internal deletion comprises: (i) the internal deletion of SEQ ID NO: 7 or SEQ ID NO: 8; or (ii) the internal deletion which encodes a protein corresponding to the protein of SEQ ID NO: 9 and/or SEQ ID NO: 10.
32. The biological sample of any one of embodiments 26-31, wherein said sample lacks a nucleic acid comprising the wild-type allele of the soybean FT1a gene of SEQ ID NO: 3 or an allelic variant thereof, the wild-type allele of the soybean JAG1 gene of SEQ ID NO: 18, the wild-type allele of the BS1 gene of SEQ ID NO: 25, the wild-type allele of the BS2 gene of SEQ ID NO: 26, and/or the wild-type allele of the TFL1b gene of SEQ ID NO: 22.
33. A polynucleotide comprising SEQ ID NO: 4, SEQ ID NO: 5, SEQ ID NO: 7, or SEQ ID NO: 8, optionally wherein the polynucleotide is isolated.
34. A polynucleotide encoding the polypeptide of SEQ ID NO: 6, SEQ ID NO: 9, SEQ ID NO: 10, 15, 16, 19, 20, 31, 32, 33, 34, or 35, optionally wherein the polynucleotide is isolated.
35. A method of producing a soybean seed lot comprising: (i) growing a population of soybean plants comprising the soybean plant of any one of embodiments 22-25; and (ii) harvesting seed from the population of soybean plants of step (i) at maturity, thereby producing the soybean seed lot.
36. The method of embodiment 35, wherein the total harvested seed weight per acre or hectare of the population of the soybean plants is increased in comparison to total harvested seed weight per acre or hectare of a population of wild-type control soybean plants lacking the loss-of-function allele.
37. The method of embodiment 35 or embodiment 36, wherein the total harvested seed weight per acre or hectare of the population of the soybean plants grown under stress is increased in comparison to total harvested seed weight per acre or hectare of a population of wild-type control soybean plants lacking the loss-of-function allele grown under stress, optionally wherein the stress comprises abiotic stress.
38. The method of any one of embodiments 35-37, wherein the average weight of 1000 seeds of the soybean seed lot is equivalent to the average weight of 1000 seeds of a wild-type control soybean seed lot lacking the loss-of-function allele.
39. The method of any one of embodiments 35-38, wherein at least 50%, 70%, 80%, or 90% of plants in the population of soybean plants have 95% of their pods at full maturity color when harvested in step (i).
40. The method of any one of embodiments 35-39, wherein the population of soybean plants are a full season variety for the soybean maturity group zone where they are grown and wherein the seed are harvested at or after a full growing season for the full season variety.
41. A method of producing a soybean crop comprising planting the seed lot of any one of embodiments 19-21.
42. The method of embodiment 41, further comprising harvesting seed from soybean crop grown from the planted seed.
43. The method of embodiment 41 or embodiment 42, wherein the total harvested seed per acre or hectare of the soybean crop is increased in comparison to the total harvested seed per acre or hectare of a wild-type control soybean crop lacking the loss-of-function allele.
44. The method of any one of embodiments 41-43, wherein the total harvested seed per acre or hectare of the soybean crop grown under stress is increased in comparison to the total harvested seed per acre or hectare of a wild-type control soybean crop lacking the loss-of-function allele grown under stress, optionally wherein the stress comprises abiotic stress.
45. The method of any one of embodiments 41-44, wherein the average weight of 1000 seeds of the harvested seed is equivalent to the average weight of 1000 seeds of harvested wild-type control soybean seed lacking the loss-of-function allele.
46. The method of any one of embodiments 41 to 45, wherein the seed are planted on or within a week of the earliest initial planting date provided by the USDA Risk Management Agency for the maturity zone where they are planted.
47. The method of any one of embodiments 41 to 46, wherein at least 50%, 70%, 80%, or 90% of plants in the soybean crop have 95% of their pods at full maturity color when harvested.
48. The method of any one of embodiments 41 to 47, wherein the soybean crop is a full season variety for the soybean maturity group zone where it is grown and wherein the seed are harvested at or after a full growing season for the full season variety.
49. A guide RNA molecule comprising a spacer RNA molecule which targets exon 1 of the FT1a gene of SEQ ID NO: 3 or an allelic variant thereof, optionally wherein the spacer RNA molecule comprises the RNA encoded by SEQ ID NO: 11 or a guide RNA molecule which targets the soybean BS1 and BS2 genes which comprises a spacer RNA encoded by SEQ ID NO: 17.
50. A method for generating a soybean plant cell of any one of embodiments 1-12, soybean plant part of any one of embodiments 13-18, or soybean plant of any one of embodiments 22-25 comprising introducing a loss-of-function allele in the endogenous soybean FT1a gene of SEQ ID NO: 3 or an allelic variant thereof.
51. The method of embodiment 50, wherein the loss-of-function allele is introduced by: (i) directing both: (a) a guide RNA (gRNA) molecule comprising a spacer RNA molecule which targets the endogenous FT1a gene of SEQ ID NO: 3 or an allelic variant thereof, exon 1 of the FT1a gene of SEQ ID NO: 3 or an allelic variant thereof, or a spacer RNA molecule comprising the RNA encoded by SEQ ID NO: 11; and (b) an RNA dependent endonuclease (RDE) which recognizes the gRNA molecule to the genome of a target soybean plant cell; and (ii) isolating a soybean plant cell, soybean plant part, or soybean plant comprising a loss-of-function allele of the endogenous soybean FT1a gene of SEQ ID NO: 3 or an allelic variant thereof.
52. The method of embodiment 51, wherein the directing of the gRNA and the RDE to the genome of the target soybean plant cell comprises introducing the gRNA, the RDE, a gRNA/RDE complex, a nucleic acid encoding the gRNA, and/or a nucleic acid encoding the RDE into the target soybean plant cell.
53. The method of embodiment 51 or embodiment 52, wherein said soybean plant cell, soybean plant part, or soybean plant comprising a loss-of-function allele is identified by: (i) analyzing a polynucleotide comprising a portion of SEQ ID NO: 3 or an allelic variant thereof encoded by a portion of SEQ ID NO: 3 or an allelic variant thereof in one or more candidate plant cells, plant parts, or plants; (ii) analyzing a polypeptide encoded by a portion of SEQ ID NO: 3 in one or more candidate plant cells, plant parts, or plants; and/or (iii) analyzing pod count per plant; seed count per plant; and/or total harvested seed weight per plant in one or more candidate plants, wherein an increased in pod count per plant; seed count per plant; and/or total harvested seed weight per plant in comparison to a wild-type control soybean plant lacking the loss-of-function allele is indicative of a soybean plant cell, soybean plant part, or soybean plant comprising the loss-of-function allele.
54. The method of embodiment 45, wherein the loss-of-function allele is introduced by crossing a soybean plant comprising the loss-of-function allele with a second soybean plant and harvesting F1 seed comprising the loss-of-function allele, thereby producing progeny soybean seed comprising the loss-of-function allele.
55. A method for generating a soybean plant cell of any one of embodiments 1-12, soybean plant part of any one of embodiments 13-18, or soybean plant of any one of embodiments 22-25 comprising: (i) screening a population of soybean plant cells, parts, or plants for the presence of a loss-of-function allele in the endogenous soybean FT1a gene of SEQ ID NO: 3 or an allelic variant thereof; and (ii) isolating a soybean plant cell, soybean plant part, or soybean plant comprising a loss-of-function allele of the soybean FT1a gene of SEQ ID NO: 3 or an allelic variant thereof.
56. The method of embodiment 55, wherein the population of soybean plant cells, parts, or plants have been subjected to one or more mutagenesis treatments, optionally wherein the mutagenesis procedure comprises chemical mutagenesis.
57. The method of embodiment 55 or embodiment 56, wherein said screening comprises: (i) analyzing a polynucleotide comprising a portion of SEQ ID NO: 3 or an allelic variant thereof or analyzing an RNA encoded by a portion of SEQ ID NO: 3 or an allelic variant thereof from one or more candidate plant cells, plant parts, or plants, wherein an insertion, deletion, and/or substitution of one or more nucleotides in said polynucleotide or RNA is indicative of the presence of the loss-of-function allele; and/or (ii) analyzing a polypeptide encoded by SEQ ID NO: 3, a portion thereof, or an allelic variant thereof from one or more candidate plant cells, plant parts, or plants, wherein an insertion, deletion, and/or substitution of one or more amino acid residues of the polypeptide or a change in the biologic or biochemical activity of the polypeptide is indicative of the presence of the loss-of-function allele.
58. The method of any one of embodiments 55-57, wherein said screening further comprising analyzing pod count per plant, seed count per plant, and/or total harvested seed weight per plant in one or more candidate plants, wherein an increase in pod count per plant, seed count per plant, and/or total harvested seed weight per plant in comparison to a wild-type control soybean plant lacking the loss-of-function allele is indicative of a soybean plant cell, soybean plant part, or soybean plant comprising the loss-of-function allele.
59. The method of any one of embodiments 55-58, wherein said screening is conducted on a population of plants grown under stress.
60. The method of embodiment 59, wherein the stress comprises abiotic stress.
61. A method for determining whether a soybean plant cell, plant part, or plant comprises a loss-of-function allele of the endogenous soybean FT1a gene of SEQ ID NO: 3 or an allelic variant thereof, the method comprising: (i) analyzing a polynucleotide comprising a portion of SEQ ID NO: 3 or an allelic variant thereof or analyzing an RNA encoded by a portion of SEQ ID NO: 3 or an allelic variant thereof from the plant cell, plant part, or plant, wherein an insertion, deletion, and/or substitution of one or more nucleotides in said polynucleotide or RNA is indicative of the presence of the loss-of-function allele; and/or (ii) analyzing a polypeptide encoded by SEQ ID NO: 3, a portion thereof, or an allelic variant thereof from the soybean plant cell, plant part, or plant, wherein an insertion, deletion, and/or substitution of one or more amino acid residues of the polypeptide or a change in the biologic or biochemical activity of the polypeptide is indicative of the presence of the loss-of-function allele.
62. The method of embodiment 61, wherein the method further comprises analyzing pod count per plant, seed count per plant, and/or total harvested seed weight per plant in one or more of the soybean plants, wherein an increase in pod count per plant, seed count per plant, and/or total harvested seed weight per plant in comparison to a wild-type control soybean plant lacking the loss-of-function allele is indicative of a soybean plant cell, soybean plant part, or soybean plant comprising the loss-of-function allele.
63. The method of embodiment 61 or 62, wherein said analyzing is conducted on a population of plants grown under stress.
64. The method of embodiment 63, wherein the stress comprises abiotic stress.
65. The method of embodiment 64, wherein the abiotic stress comprises drought stress.

The present disclosure also provides for soybean plants and plant parts comprising a mutated BS1 or mutated BS2 gene which can optionally further comprise a loss-of-function allele of an FT1a, JAG1, and/or TFL1b gene, along with methods of making and using the same. Additional non-limiting embodiments of the disclosure are provided herein as follows:
1. A soybean plant cell comprising:
(i) an amorphic allele of the soybean BS1 gene of SEQ ID NO: 25 or an allelic variant thereof, wherein said BS1 gene or variant thereof is in the chromosomal location of the wild-type soybean BS1 gene; or (ii) an amorphic allele of the soybean BS2 gene of SEQ ID NO: 26 or an allelic variant thereof, wherein said BS2 gene or allelic variant thereof is in the chromosomal location of the wild-type soybean BS2 gene; optionally wherein the soybean plant cell further comprises (iii) a loss-of-function allele of the endogenous soybean FT1a gene of SEQ ID NO: 3 or an allelic variant thereof; (iv) a loss-of-function allele of an endogenous soybean JAG1 gene of SEQ ID NO: 18 or an allelic variant thereof; and/or (v) a loss-of-function allele of an endogenous soybean TFL1b gene of SEQ ID NO: 22 or an allelic variant thereof; optionally wherein the amorphic or loss-of-function alleles are non-natural alleles.
2. The soybean plant cell of embodiment 1, wherein the plant cell is homozygous for the amorphic allele in the soybean BS2 gene of SEQ ID NO: 26 or allelic variant thereof.
3. The soybean plant cell of embodiment 1 or 2, wherein the plant cell homozygous for the amorphic allele in the soybean BS2 gene of SEQ ID NO: 26 or allelic variant thereof comprises at least one isomorphic allele of the soybean BS1 gene of SEQ ID NO: 25 or an allelic variant thereof.
4. The soybean plant cell of any one of embodiments 1 to 3, wherein the amorphic allele of said BS2 gene comprises a frameshift, nonsense, and/or deletion mutation in said BS1 gene encoding a truncated BS2 protein having 53 or less of the amino terminal amino acid residues corresponding to residues 1 to 53 of the wild-type BS2 protein of SEQ ID NO: 28.
5. The soybean plant cell of embodiment 4, wherein the amorphic allele of said BS2 gene encodes the polypeptide of SEQ ID NO: 29 or comprises the polynucleotide of SEQ ID NO: 31, SEQ ID NO: 38, or an allelic variant thereof.
6. The soybean plant cell of embodiment 1, wherein the plant cell is homozygous for the amorphic allele in the soybean BS1 gene of SEQ ID NO: 25 or allelic variant thereof.
7. The soybean plant cell of embodiment 6, wherein the plant cell homozygous for the amorphic allele in the soybean BS1 gene of SEQ ID NO: 25 or allelic variant thereof comprises at least one isomorphic allele of the soybean BS2 gene of SEQ ID NO: 26 or an allelic variant thereof.
8. The soybean plant cell of embodiment 6 or 7, wherein the amorphic allele comprises a frameshift, nonsense, and/or deletion mutation in said BS1 gene encoding a truncated BS1 protein having about 53 or less of the amino terminal amino acid residues corresponding to residues 1 to 53 of a wild-type BS1 protein of SEQ ID NO: 27.
9. The soybean plant cell of embodiment 8, wherein the amorphic allele of said BS1 gene comprises the polynucleotide of SEQ ID NO: 32 or an allelic variant thereof.
10. The soybean plant cell of any one of embodiments 1 to 9, wherein: (i) said amorphic allele of the soybean BS1 gene of SEQ ID NO: 25 or an allelic variant thereof is a non-naturally occurring amorphic allele of said soybean BS1 gene of SEQ ID NO: 25 or an allelic variant thereof; or (ii) said amorphic allele of the soybean BS2 gene of SEQ ID NO: 26 or an allelic variant thereof is a non-naturally occurring amorphic allele of the soybean BS2 gene of SEQ ID NO: 26 or an allelic variant thereof.
11. The soybean plant cell of any one of embodiments 1 to 10, wherein:
(i) the allelic variant of SEQ ID NO: 25 comprises a DNA sequence having at least 95%, 96%, 97%, 98%, or 99% sequence identity to SEQ ID NO: 25 or wherein the allelic variant of SEQ ID NO: 26 comprises a DNA sequence having at least 95%, 96%, 97%, 98% or 99% sequence identity to SEQ ID NO: 26;
(ii) the soybean plant cell further comprises an A2704-12, A5547-127, BPS-CV127-9, DAS44406-6, DAS68416-4, DAS81419-2, DP305423, GTS 40-3-2, HOS, A5547-127, MON87701, MON87705, MON87708, MON87769, MON89788, MON98788, MST-FGØ72-3, or SYHT0H210 transgenic event or modification thereof;
(iii)) the loss-of-function allele of the FT1a gene comprises an internal deletion comprising: (a) at least nucleotides corresponding to nucleotides 318 to 345, 319 to 356, 320 to 352, 321 to 348, 322 to 350, 323 to 328, 324 to 328, 325 to 347, 326 to 349, 327 to 347, 328 to 348, 329 to 347, 330 to 348, 331 to 347, 332 to 347, 333 to 347, 334 to 345, 335 to 347, 336 to 347, 337 to 347, 338 to 345, 339 to 345, 340, 341 to 345, 342 to 344, 343 to 347, 343 to 351, 344 to 345, 345 to 347, 346 to 347, 347 to 350, 348 to 351, or 331 to 371 of SEQ ID NO: 3 or an allelic variant thereof; (b) at least nucleotides encoding amino acids corresponding to N39 to C41 of SEQ ID NO: 1 or SEQ ID NO: 2; (c) at least nucleotides corresponding to a deletion of an FT1a gene set forth in Table 2; or (d) at least nucleotides corresponding to a deletion of an FT1a gene set forth in SEQ ID NO: 13, 21, or an allelic variant thereof; and/or
(iv) the loss-of-function allele of the JAG1 gene comprises an internal deletion comprising: (i) at least nucleotides corresponding to a deletion of a JAG1 gene set forth in Table 3; (ii) at least nucleotides corresponding to a deletion of a JAG1 gene set forth in SEQ ID NO: 15, 16, 19, 20, or an allelic variant thereof; or (iii) at least nucleotides corresponding to nucleotides 408 to 426, 409 to 427, 410 to 429, 411 to 428, 412 to 428, 413 to 427, 414 to 426, 415 to 427, 416 to 427, 417 to 426, 418 to 426, 419 to 426, 420 to 426, 421 to 426, 422 to 426, 423 to 426, 424 to 427, 425 to 427, 426 to 429, 427 to 428, 429 to 430, 430, or 431 of SEQ ID NO: 18 or an allelic variant thereof.
12. A soybean plant part comprising the soybean plant cell of any one of embodiments 1 to 11.
13. The soybean plant part of embodiment 12, wherein the part is a stem, root, leaf, flower, pod, or seed.
14. The soybean plant part of embodiment 12 or 13, wherein the part is a seed and wherein the weight of the seed is increased in comparison to a control seed having an isomorphic allele in both the soybean BS1 gene of SEQ ID NO: 25 or an allelic variant of SEQ ID NO: 25 and in the soybean BS2 gene of SEQ ID NO: 26 or an allelic variant of SEQ ID NO: 26.
15. The soybean plant part of any one of embodiments 12 to 14, wherein the part is a seed and wherein the average weight of the seed is increased by at least 10% in comparison to the control seed.
16. The soybean plant part of embodiment 15, wherein the part is a seed and wherein the average weight of the seed is increased by 18% to 28% in comparison to the control seed.
17. A soybean seed lot comprising the seed of any one of embodiments 13 to 16.
18. The soybean seed lot of embodiment 17, wherein the average weight of 1000 seeds in the seed lot is increased by at least 17% in comparison to a control seed lot lacking the amorphic allele of the BS1 or the BS2 gene.
19. The soybean seed lot of embodiment 17, wherein the average number of seeds per kilogram of seeds in the seed lot is decreased by at least 15% in comparison to a control seed lot lacking the amorphic allele of the BS1 or the BS2 gene.
20. A soybean plant comprising the soybean plant cell of any one of embodiments 1 to 11.
21. The soybean plant of embodiment 20, wherein the soybean plant yields seed having increased seed weight in comparison to a control seed lacking the amorphic allele of the soybean BS1 gene of SEQ ID NO: 25 or an allelic variant thereof or control seed lacking the amorphic allele in the soybean BS2 gene of SEQ ID NO: 26 or an allelic variant thereof.
22. The soybean plant of embodiment 20 or 21, wherein the average weight of the seed is increased by at least 10% in comparison to the control seed.
23. A biological sample comprising of a soybean plant comprising a nucleic acid comprising an amorphic allele of the soybean BS1 gene of SEQ ID NO: 25 or an allelic variant thereof; and/or an amorphic allele of the soybean BS2 gene of SEQ ID NO: 26 or an allelic variant thereof, optionally wherein the biological sample further comprises a nucleic acid containing a loss-of-function allele of the soybean FT1a gene of SEQ ID NO: 3 or an allelic variant thereof, a loss-of-function allele of the soybean JAG1 gene of SEQ ID NO: 18 or an allelic variant thereof, a loss-of-function allele of an endogenous soybean TFL1b gene of SEQ ID NO: 22 or an allelic variant thereof, and/or a loss-of-function allele of an endogenous soybean BS2 gene of SEQ ID NO: 26 or an allelic variant thereof.
24. The biological sample of embodiment 23, wherein the sample comprises seed meal or a tissue sample homogenate, optionally wherein the tissue sample comprises a sample of leaf, flower, pod, seed, stem, or root tissue.
25. The biological sample of embodiment 23 or 24, wherein the amorphic allele comprises a frameshift, nonsense, and/or deletion mutation in said BS2 gene encoding a truncated BS2 protein having 53 or less of the amino terminal amino acid residues corresponding to residues 1 to 53 of the wild-type BS2 protein of SEQ ID NO: 28, optionally wherein the amorphic allele of said BS2 gene encodes the polypeptide of SEQ ID NO: 29 or SEQ ID NO: 30 or an allelic variant thereof or comprises the polynucleotide of SEQ ID NO: 31, 34, 35, 38, or an allelic variant thereof.
26. The biological sample of embodiment 23, 24, or 25 wherein said sample lacks nucleic acids comprising an isomorphic or hypomorphic allele of the soybean BS2 gene of SEQ ID NO: 26 or an allelic variant thereof.
27. The biological sample of embodiment 23, 24, 25, or 26, wherein the amorphic allele comprises a frameshift, nonsense, and/or deletion mutation in said BS1 gene encoding a truncated BS1 protein having 53 or less of the amino terminal amino acid residues corresponding to residues 1 to 53 of a wild-type BS1 protein of SEQ ID NO: 27, optionally wherein the amorphic allele of said BS1 gene comprises the polynucleotide of SEQ ID NO: 32 or an allelic variant thereof.
28. The biological sample of embodiment 23, 24, or 27, wherein said sample lacks nucleic acids comprising an isomorphic allele of the soybean BS1 gene of SEQ ID NO: 25 or an allelic variant thereof.
29. A polynucleotide comprising the DNA molecule of SEQ ID NO: 31, SEQ ID NO: 32, SEQ ID NO: 33, SEQ ID NO: 34, SEQ ID NO: 35, or SEQ ID NO: 38.
30. A polynucleotide encoding the polypeptide of SEQ ID NO: 29, SEQ ID NO: 30, or an allelic variant thereof.
31. A method of producing a soybean seed lot comprising: (i) growing a population of soybean plants comprising the soybean plant of any one of embodiments 20 to 22; and (ii) harvesting seed from the population of soybean plants of step (i) at maturity, thereby producing the soybean seed lot.
32. The method of embodiment 31 wherein the average weight of 1000 seeds in the seed lot is increased by at least 17% in comparison to a control seed lot lacking the amorphic allele of the BS1 or the BS2 gene.
33. The method of embodiment 31, wherein the average number of seeds per kilogram of seeds in the seed lot is decreased by at least 15% in comparison to a control seed lot lacking the amorphic allele of the BS1 or the BS2 gene.
34. A method of producing a soybean crop comprising planting the seed lot of any one of embodiments 17 to 19.
35. The method of embodiment 34, further comprising harvesting seed from soybean plants grown from the planted seed.
36. The method of embodiment 35, wherein the average weight of 1000 seeds in the harvested seed is increased by at least 17% in comparison to a control seed lot lacking the amorphic allele of the BS1 or the BS2 gene.
37. The method of embodiment 35, wherein the average number of seeds per kilogram of seeds in the harvested seed is decreased by at least 15% in comparison to a control seed lot lacking the amorphic allele of the BS1 or the BS2 gene.
38. A guide RNA molecule comprising from its 5′ to 3′ end a Cas12a direct repeat which is operably linked to a spacer RNA molecule encoded by the DNA molecule of SEQ ID NO: 36.
39. A method for generating a soybean plant cell, soybean plant part, or soybean plant of any one of embodiments 1 to 22 comprising; (i) directing a guide RNA (gRNA) molecule comprising a spacer RNA molecule encoded by the DNA molecule of SEQ ID NO: 36 and an RNA dependent endonuclease (RDE) which recognizes the gRNA molecule to the genome of a target soybean plant cell comprising an isomorphic allele of the soybean BS1 gene of SEQ ID NO: 25, the soybean BS2 gene of SEQ ID NO: 26, or an allelic variant thereof; and (ii) isolating a soybean plant cell, soybean plant part, or soybean plant comprising an amorphic allele of the soybean BS1 gene of SEQ ID NO: 25 or an allelic variant thereof, wherein said BS1 gene or variant thereof is in the chromosomal location of the wild-type soybean BS1 gene; or a soybean plant cell, soybean plant part, or soybean plant comprising an amorphic allele of the soybean BS2 gene of SEQ ID NO: 26 or an allelic variant thereof, wherein said BS2 gene or allelic variant thereof is in the chromosomal location of the wild-type soybean BS2 gene.
40. The method of embodiment 39, wherein the directing of the gRNA and the RDE to the genome of the target soybean plant cell comprises introducing the gRNA, the RDE, a gRNA/RDE complex, a nucleic acid encoding the gRNA, and/or a nucleic acid encoding the RDE into the target soybean plant cell.
41. The method of embodiment 39 or 40, wherein the RDE comprises a Cas12 RDE.
42. The method of embodiment 39, 40, or 41, wherein the isolating comprises screening a population of soybean plant cells, soybean plant parts, or soybean plants for a deletion in soybean genomic DNA located at or between residues 890 to 916 of SEQ ID NO: 25 and/or at or between residues 1578 to 1582 of SEQ ID NO: 26.
43. The method of embodiment 39, 40, or 41, wherein the isolating comprises screening progeny soybean plants for a soybean plant which yields seed having increased seed weight in comparison to a control seed lacking the amorphic allele of the soybean BS1 gene of SEQ ID NO: 25 or an allelic variant thereof or control seed lacking the amorphic allele in the soybean BS2 gene of SEQ ID NO: 26 or an allelic variant thereof.

### EXAMPLES

### Example 1. Generation of Soybean with a Mutated FT1a Gene

A vector was created to transform soybean plants and disrupt the open reading frame of the GmFTA1a gene (Glyma10g38970; SEQ ID NO: 3) through CRISPR-mediated gene editing. A CRISPR guide RNA comprising a crRNA fused to a spacer RNA encoded by SEQ ID NO: 11 was designed to target exon 1 of the Glycine max FT1a gene.

Genotypes recovered included −10:13D and −4:9D. The −10:13D genotype has a 13 bp deletion (SEQ ID NO: 5) resulting in a frameshift mutation and introducing a premature stop codon. This −10:13D genotype is predicted to encode a truncated 84 amino acid polypeptide (SEQ ID NO: 6). The −4:9D genotype has a 9 bp deletion (SEQ ID NO: 8) predicted to encode a polypeptide with a 3 amino acid internal deletion (SEQ ID NOs: 9 and 10).

### Example 2. Performance of Soybean with a Mutated FT1a Gene

Seeds of the homozygous −4:9D mutant line SENF2228 and the homozygous −10:13D mutant line SENF2229 were increased and planted in rows in the field along with checks and lines having unrelated edits. Data on total yield from field trials showed a tendency towards higher yield per plot for SENF2228 and SENF2229 compared to adjacently grown wild types, null segregants, and some unrelated mutants.

Some field-grown plants were also subjected to more detailed phenotyping. SENF2228 and SENF2229 showed a consistent tendency towards higher yield when compared to controls grown at comparable densities. This trend was apparent in yield components such as pod count, seed count, and total seed weight, though the individual seed weight was comparable to that of null segregant and wild type checks.

### Example 3. Performance of Soybean with a Mutated FT1a Gene in Field Test

Seeds of the homozygous −4:9D mutant line SENF2228 and the homozygous −10:13D mutant line SENF2229 were increased and planted in rows in the field along with a NINF1170 check. The results of the field test are shown in Table 1. Phenotypic characteristics of SENF2228, SENF2229 and comparison variety NINF1170 are shown in Table 1.

TABLE 1

| | SENF2228 | SENF2229 | |
|---|---|---|---|
| Phenotype | (−4:9D) | (−10:13D) | NINF1170 |
| Seed Yield ¹ | 53.0 | 52.9 | 49.6 |
| Plants Per Acre ² | 123554 | 123568 | 105746 |
| Plant Height ³ | 30.9 | 30.4 | 31.0 |
| Time To Flowering ⁴ | 44 | 43 | 41 |
| Time to Beginning Pod | 61 | 60 | 59 |
| Time to Beginning Seed ⁶ | 71 | 69 | 69 |
| Time to Full Maturity ⁷ | 117 | 115 | 117 |

¹ “Seed Yield” refers to the yield adjusted to 12% moisture in bushels/acre of the grain at harvest.
² “Plants Per Acre” refers to the estimated number of soybean plants per acre.
³ “Plant height” refers to the plant height taken from soil level to the apical node on the main stem of the plant at maturity and is measured in inches.
⁴ “Time to Flowering” refers to the number of days from planting when 50% of the plants have at least one open flower at any node on the main stem
⁵ “Time to Beginning Pod” refers to the number of days from planting when 50% of plants had pods 3/16 inch long at one of the four uppermost nodes on the main stem with a fully developed leaf.
⁶ “Time to Beginning Seed” refers to the number of days from planting when 50% of the plants have seed ⅛ inch long in a pod at one of the four uppermost nodes on the main stem with a fully developed leaf.
⁷ “Time to Full Maturity” refers to the number of days from planting when 50% of the plants had 95% of their pods reach full maturity color.

### Example 4. Generation of Additional Soybean Plants with a Mutated FT1a Gene and a Mutated JAG1 Gene

Additional soybean plants comprising loss-of-function mutations in the FT1a gene were generated essentially as described in Example 1 using a gRNA comprising the spacer RNA encoded by SEQ ID NO: 11 and a type V Cas nuclease. Soybean plants generated included the SENF2225 and SENF2226 plants which contain the −16:41D deletion in the FT1a gene shown in FIGS. 5 and 6. Sequences of the soybean genomic DNA comprising the −16:41D deletion in the FT1a gene are provided in SEQ ID NO: 13 (i.e., a segment of the FT1a gene containing the deletion) and SEQ ID NO: 21 (i.e., the FT1a gene containing the deletion).

Additional deletions in the soybean FT1a gene in a variety of different soybean genotypes generated with the gRNA comprising the spacer RNA encoded by SEQ ID NO: 11 and the type V Cas nuclease are provided in Table 2, which is incorporated herein by reference in its entirety. Additional deletions in the soybean JAG1 gene in a variety of different soybean genotypes generated with the gRNA comprising the spacer RNA encoded by SEQ ID NO: 17 and the type V Cas nuclease are provided in Table 3, which is incorporated herein by reference in its entirety. The left-most column of Tables 2 and 3 labelled “variant_ID” provides the coordinates of the different FT1a and JAG1 gene deletions (respectively) relative to the cleavage site in the soy FT1a gene DNA specified by the gRNA comprising the spacer RNA encoded by SEQ ID NO: 11 and relative to the cleavage site in the soy JAG1 gene specified by the gRNA comprising the spacer RNA encoded by SEQ ID NO: 17 (respectively). The cleavage site for the type V Cas nuclease (e.g., a Cas12 nuclease) is located at a position in the gRNA spacer (e.g., encoded by SEQ ID NO: 11 or 17) that is 19 nucleotides away from the PAM site. Different deletions in the target FT1a and JAG1 genes can fall either 5′ or 3′ to the cleavage site specified by the gRNA. In the variant_ID of column 1, the first number represents either the number of deleted nucleotides 5′ of the cleavage site (negative (−) values) or 3′ of the cleavage site (positive values (+)) for deletions which extend either 5′ or 3′ of the cleavage site. The cleavage site in the FT1a gene specified by the spacer encoded by SEQ ID NO: 11 is located between nucleotides 346 (“−1”) and 347 (“+1”) in the FT1a SEQ ID NO: 3 reference sequence. The cleavage site in the JAG1 gene specified by the spacer encoded by SEQ ID NO: 17 is located between nucleotides 425 (“−1”) and 426 (“+1”) in the JAG1 SEQ ID NO: 18 reference sequence. The second number in the variant_ID represents the total number of nucleotides which have been deleted. This nomenclature used in Tables 2 and 3 is illustrated in FIGS. 5 and 6 for: (i) the FT1a deletion “−16:41D” generated with the gRNA comprising the spacer RNA encoded by SEQ ID NO: 11 and the type V Cas nuclease; and (ii) the JAG1 “−9:13D” and “1:6D” deletions generated with the gRNA comprising the spacer RNA encoded by SEQ ID NO: 17 and the type V Cas nuclease. The second column (“Deletion size”) indicates the number of deleted nucleotides. For Table 2, the third column indicates the deleted nucleotides which correspond to the deletion in a wild-type FT1a reference sequence of SEQ ID NO: 3. For Table 3, the third column indicates the deleted nucleotides which correspond to the deletion in a wild-type JAG1 reference sequence of SEQ ID NO: 18. Since the deletions disclosed in Tables 2 and 3 were generated in a number of different soybean genotypes, it is anticipated that certain deletions shown in those Tables have been generated in an allelic variant of the soybean FT1a gene of SEQ ID NO: 3 or in an allelic variant of the soybean JAG1 gene of SEQ ID NO: 18. In certain embodiments, such allelic variants of SEQ ID NO: 3 and SEQ ID NO: 18 can have at least 90%, 95%, 97%, 98%, or 99% nucleotide sequence identity to SEQ ID NO: 3 and SEQ ID NO: 18.

The SENF2225 and SENF2226 plants also contained loss-of-function mutations in the soybean JAG1 gene as shown in FIGS. 5 and 6. The JAG1 mutants were generated essentially as described in WO2023/183772, which is incorporated herein by reference in its entirety. Deletions in JAG1 were generated with a type V Cas nuclease and a CRISPR guide RNA comprising a crRNA fused to the RNA encoded by the JAG1_g3 (SEQ ID NO: 17) spacer. The SENF2225 plants contained the −9:13D JAG1 gene deletion set forth in SEQ ID NO: 15 (i.e., a segment of the JAG1 gene containing the −9:13D deletion) and SEQ ID NO: 19 (i.e., the JAG1 gene containing the −9:13D deletion). The SENF2226 plants contained the 1:6D JAG1 gene deletion set forth in SEQ ID NO: 16 (i.e., a segment of the JAG1 gene containing the 1:61D deletion) and SEQ ID NO: 20 (i.e., the JAG1 gene containing the 1:6D) deletion).

TABLE 2
Additional Deletions in the FT1a gene

| | | Deleted Nucleotides in the |
|---|---|---|
| Variant | Deletion Size | corresponding FT1a gene SEQ |
| ID | (nucleotides) | ID NO: 3 reference sequence |
| −16:41D | 41 | 331-371 |
| −10:11D | 11 | 337-347 |
| −10:12D | 12 | 337-348 |
| −10:13D | 13 | 337-349 |
| −10:14D | 14 | 337-350 |
| −10:15D | 15 | 337-351 |
| −10:16D | 16 | 337-352 |
| −10:18D | 18 | 337-354 |
| −10:19D | 19 | 337-355 |
| −10:20D | 20 | 337-356 |
| −10:21D | 21 | 337-357 |
| −10:25D | 25 | 337-361 |
| −10:26D | 26 | 337-362 |
| −10:27D | 27 | 337-363 |
| −10:28D | 28 | 337-364 |
| −10:30D | 30 | 337-366 |
| −10:35D | 35 | 337-371 |
| −10:37D | 37 | 337-373 |
| −10:38D | 38 | 337-374 |
| −10:39D | 39 | 337-375 |
| −10:43D | 43 | 337-379 |
| −11:12D | 12 | 336-347 |
| −11:13D | 13 | 336-348 |
| −11:14D | 14 | 336-349 |
| −11:15D | 15 | 336-350 |
| −11:16D | 16 | 336-351 |
| −11:17D | 17 | 336-352 |
| −11:19D | 19 | 336-354 |
| −11:20D | 20 | 336-355 |
| −11:21D | 21 | 336-356 |
| −11:22D | 22 | 336-357 |
| −11:28D | 28 | 336-363 |
| −11:31D | 31 | 336-366 |
| −11:40D | 40 | 336-375 |
| −11:43D | 43 | 336-378 |
| −11:45D | 45 | 336-380 |
| −12:13D | 13 | 335-347 |
| −12:15D | 15 | 335-349 |
| −12:16D | 16 | 335-350 |
| −12:17D | 17 | 335-351 |
| −12:18D | 18 | 335-352 |
| −12:19D | 19 | 335-353 |
| −12:20D | 20 | 335-354 |
| −12:24D | 24 | 335-358 |
| −12:25D | 25 | 335-359 |
| −12:30D | 30 | 335-364 |
| −12:33D | 33 | 335-367 |
| −12:34D | 34 | 335-368 |
| −12:42D | 42 | 335-376 |
| −13:12D | 12 | 334-345 |
| −13:14D | 14 | 334-347 |
| −13:15D | 15 | 334-348 |
| −13:17D | 17 | 334-350 |
| −13:19D | 19 | 334-352 |
| −13:20D | 20 | 334-353 |
| −13:21D | 21 | 334-354 |
| −13:22D | 22 | 334-355 |
| −13:24D | 24 | 334-357 |
| −13:25D | 25 | 334-358 |
| −13:27D | 27 | 334-360 |
| −13:28D | 28 | 334-361 |
| −13:29D | 29 | 334-362 |
| −13:30D | 30 | 334-363 |
| −13:32D | 32 | 334-365 |
| −13:33D | 33 | 334-366 |
| −13:34D | 34 | 334-367 |
| −13:39D | 39 | 334-372 |
| −13:43D | 43 | 334-376 |
| −13:47D | 47 | 334-380 |
| −14:15D | 15 | 333-347 |
| −14:17D | 17 | 333-349 |
| −14:18D | 18 | 333-350 |
| −14:19D | 19 | 333-351 |
| −14:20D | 20 | 333-352 |
| −14:21D | 21 | 333-353 |
| −14:22D | 22 | 333-354 |
| −14:24D | 24 | 333-356 |
| −14:26D | 26 | 333-358 |
| −14:27D | 27 | 333-359 |
| −14:28D | 28 | 333-360 |
| −14:32D | 32 | 333-364 |
| −14:38D | 38 | 333-370 |
| −14:39D | 39 | 333-371 |
| −14:45D | 45 | 333-377 |
| −14:49D | 49 | 333-381 |
| −15:16D | 16 | 332-347 |
| −15:18D | 18 | 332-349 |
| −15:19D | 19 | 332-350 |
| −15:20D | 20 | 332-351 |
| −15:21D | 21 | 332-352 |
| −15:22D | 22 | 332-353 |
| −15:23D | 23 | 332-354 |
| −15:25D | 25 | 332-356 |
| −15:27D | 27 | 332-358 |
| −15:29D | 29 | 332-360 |
| −15:33D | 33 | 332-364 |
| −16:17D | 17 | 331-347 |
| −16:19D | 19 | 331-349 |
| −16:20D | 20 | 331-350 |
| −16:22D | 22 | 331-352 |
| −16:23D | 23 | 331-353 |
| −16:24D | 24 | 331-354 |
| −16:26D | 26 | 331-356 |
| −16:28D | 28 | 331-358 |
| −16:29D | 29 | 331-359 |
| −16:30D | 30 | 331-360 |
| −16:34D | 34 | 331-364 |
| −16:40D | 40 | 331-370 |
| −16:41D | 41 | 331-371 |
| −16:50D | 50 | 331-380 |
| −16:51D | 51 | 331-381 |
| −17:19D | 19 | 330-348 |
| −17:20D | 20 | 330-349 |
| −17:22D | 22 | 330-351 |
| −17:24D | 24 | 330-353 |
| −17:27D | 27 | 330-356 |
| −17:28D | 28 | 330-357 |
| −17:29D | 29 | 330-358 |
| −17:31D | 31 | 330-360 |
| −17:32D | 32 | 330-361 |
| −17:33D | 33 | 330-362 |
| −17:35D | 35 | 330-364 |
| −17:36D | 36 | 330-365 |
| −17:41D | 41 | 330-370 |
| −17:43D | 43 | 330-372 |
| −17:45D | 45 | 330-374 |
| −18:19D | 19 | 329-347 |
| −18:20D | 20 | 329-348 |
| −18:22D | 22 | 329-350 |
| −18:24D | 24 | 329-352 |
| −18:25D | 25 | 329-353 |
| −18:26D | 26 | 329-354 |
| −18:27D | 27 | 329-355 |
| −18:29D | 29 | 329-357 |
| −18:30D | 30 | 329-358 |
| −18:31D | 31 | 329-359 |
| −18:32D | 32 | 329-360 |
| −18:34D | 34 | 329-362 |
| −18:37D | 37 | 329-365 |
| −18:38D | 38 | 329-366 |
| −18:51D | 51 | 329-379 |
| −19:21D | 21 | 328-348 |
| −19:23D | 23 | 328-350 |
| −19:25D | 25 | 328-352 |
| −19:27D | 27 | 328-354 |
| −19:28D | 28 | 328-355 |
| −19:30D | 30 | 328-357 |
| −19:31D | 31 | 328-358 |
| −19:34D | 34 | 328-361 |
| −19:36D | 36 | 328-363 |
| −1:11D | 11 | 346-356 |
| −1:13D | 13 | 346-358 |
| −1:15D | 15 | 346-360 |
| −1:23D | 23 | 346-368 |
| −1:29D | 29 | 346-374 |
| −1:2D | 2 | 346-347 |
| −1:32D | 32 | 346-377 |
| −1:36D | 36 | 346-381 |
| −1:39D | 39 | 346-384 |
| −1:44D | 44 | 346-389 |
| −1:4D | 4 | 346-349 |
| −1:5D | 5 | 346-350 |
| −1:6D | 6 | 346-351 |
| −1:7D | 7 | 346-352 |
| −1:8D | 8 | 346-353 |
| −1:9D | 9 | 346-354 |
| −20:21D | 21 | 327-347 |
| −20:22D | 22 | 327-348 |
| −20:23D | 23 | 327-349 |
| −20:24D | 24 | 327-350 |
| −20:25D | 25 | 327-351 |
| −20:28D | 28 | 327-354 |
| −20:29D | 29 | 327-355 |
| −20:31D | 31 | 327-357 |
| −20:35D | 35 | 327-361 |
| −20:36D | 36 | 327-362 |
| −20:37D | 37 | 327-363 |
| −20:47D | 47 | 327-373 |
| −21:24D | 24 | 326-349 |
| −21:25D | 25 | 326-350 |
| −21:26D | 26 | 326-351 |
| −21:27D | 27 | 326-352 |
| −21:29D | 29 | 326-354 |
| −21:32D | 32 | 326-357 |
| −22:18D | 18 | 325-342 |
| −22:23D | 23 | 325-347 |
| −22:26D | 26 | 325-350 |
| −22:28D | 28 | 325-352 |
| −22:29D | 29 | 325-353 |
| −22:30D | 30 | 325-354 |
| −22:31D | 31 | 325-355 |
| −22:33D | 33 | 325-357 |
| −22:34D | 34 | 325-358 |
| −22:35D | 35 | 325-359 |
| −22:38D | 38 | 325-362 |
| −22:41D | 41 | 325-365 |
| −22:42D | 42 | 325-366 |
| −22:43D | 43 | 325-367 |
| −22:44D | 44 | 325-368 |
| −22:51D | 51 | 325-375 |
| −23:25D | 25 | 324-348 |
| −23:26D | 26 | 324-349 |
| −23:27D | 27 | 324-350 |
| −23:28D | 28 | 324-351 |
| −23:29D | 29 | 324-352 |
| −23:31D | 31 | 324-354 |
| −23:32D | 32 | 324-355 |
| −23:40D | 40 | 324-363 |
| −23:41D | 41 | 324-364 |
| −24:26D | 26 | 323-348 |
| −24:27D | 27 | 323-349 |
| −24:28D | 28 | 323-350 |
| −24:29D | 29 | 323-351 |
| −24:30D | 30 | 323-352 |
| −24:32D | 32 | 323-354 |
| −24:33D | 33 | 323-355 |
| −24:35D | 35 | 323-357 |
| −24:39D | 39 | 323-361 |
| −24:41D | 41 | 323-363 |
| −24:42D | 42 | 323-364 |
| −24:44D | 44 | 323-366 |
| −25:29D | 29 | 322-350 |
| −25:31D | 31 | 322-352 |
| −25:33D | 33 | 322-354 |
| −25:36D | 36 | 322-357 |
| −25:37D | 37 | 322-358 |
| −25:38D | 38 | 322-359 |
| −25:44D | 44 | 322-365 |
| −25:46D | 46 | 322-367 |
| −25:47D | 47 | 322-368 |
| −25:54D | 54 | 322-375 |
| −26:28D | 28 | 321-348 |
| −26:30D | 30 | 321-350 |
| −26:35D | 35 | 321-355 |
| −26:43D | 43 | 321-363 |
| −26:45D | 45 | 321-365 |
| −27:33D | 33 | 320-352 |
| −27:35D | 35 | 320-354 |
| −27:48D | 48 | 320-367 |
| −28:38D | 38 | 319-356 |
| −29:28D | 28 | 318-345 |
| −29:35D | 35 | 318-352 |
| −29:37D | 37 | 318-354 |
| −2:10D | 10 | 345-354 |
| −2:11D | 11 | 345-355 |
| −2:12D | 12 | 345-356 |
| −2:13D | 13 | 345-357 |
| −2:17D | 17 | 345-361 |
| −2:18D | 18 | 345-362 |
| −2:19D | 19 | 345-363 |
| −2:1D | 1 | 345-345 |
| −2:20D | 20 | 345-364 |
| −2:21D | 21 | 345-365 |
| −2:22D | 22 | 345-366 |
| −2:27D | 27 | 345-371 |
| −2:28D | 28 | 345-372 |
| −2:29D | 29 | 345-373 |
| −2:30D | 30 | 345-374 |
| −2:34D | 34 | 345-378 |
| −2:3D | 3 | 345-347 |
| −2:4D | 4 | 345-348 |
| −2:5D | 5 | 345-349 |
| −2:6D | 6 | 345-350 |
| −2:7D | 7 | 345-351 |
| −2:8D | 8 | 345-352 |
| −30:32D | 32 | 317-348 |
| −30:35D | 35 | 317-351 |
| −30:37D | 37 | 317-353 |
| −30:39D | 39 | 317-355 |
| −30:47D | 47 | 317-363 |
| −31:30D | 30 | 316-345 |
| −31:33D | 33 | 316-348 |
| −31:34D | 34 | 316-349 |
| −31:36D | 36 | 316-351 |
| −31:37D | 37 | 316-352 |
| −31:39D | 39 | 316-354 |
| −31:40D | 40 | 316-355 |
| −31:41D | 41 | 316-356 |
| −32:34D | 34 | 315-348 |
| −32:38D | 38 | 315-352 |
| −32:40D | 40 | 315-354 |
| −32:43D | 43 | 315-357 |
| −33:35D | 35 | 314-348 |
| −33:36D | 36 | 314-349 |
| −33:37D | 37 | 314-350 |
| −34:39D | 39 | 313-351 |
| −34:41D | 41 | 313-353 |
| −35:33D | 33 | 312-344 |
| −35:34D | 34 | 312-345 |
| −35:38D | 38 | 312-349 |
| −35:40D | 40 | 312-351 |
| −39:43D | 43 | 308-350 |
| −3:11D | 11 | 344-354 |
| −3:12D | 12 | 344-355 |
| −3:13D | 13 | 344-356 |
| −3:14D | 14 | 344-357 |
| −3:18D | 18 | 344-361 |
| −3:19D | 19 | 344-362 |
| −3:20D | 20 | 344-363 |
| −3:21D | 21 | 344-364 |
| −3:22D | 22 | 344-365 |
| −3:23D | 23 | 344-366 |
| −3:28D | 28 | 344-371 |
| −3:29D | 29 | 344-372 |
| −3:2D | 2 | 344-345 |
| −3:30D | 30 | 344-373 |
| −3:31D | 31 | 344-374 |
| −3:32D | 32 | 344-375 |
| −3:35D | 35 | 344-378 |
| −3:36D | 36 | 344-379 |
| −3:37D | 37 | 344-380 |
| −3:40D | 40 | 344-383 |
| −3:43D | 43 | 344-386 |
| −3:4D | 4 | 344-347 |
| −3:5D | 5 | 344-348 |
| −3:6D | 6 | 344-349 |
| −3:7D | 7 | 344-350 |
| −3:8D | 8 | 344-351 |
| −3:9D | 9 | 344-352 |
| −42:50D | 50 | 305-354 |
| −42:51D | 51 | 305-355 |
| −43:47D | 47 | 304-350 |
| −43:50D | 50 | 304-353 |
| −43:52D | 52 | 304-355 |
| −49:55D | 55 | 298-352 |
| −4:10D | 10 | 343-352 |
| −4:11D | 11 | 343-353 |
| −4:12D | 12 | 343-354 |
| −4:14D | 14 | 343-356 |
| 4:16D | 16 | 343-358 |
| −4:17D | 17 | 343-359 |
| −4:18D | 18 | 343-360 |
| −4:1D | 1 | 343-343 |
| −4:22D | 22 | 343-364 |
| −4:26D | 26 | 343-368 |
| −4:27D | 27 | 343-369 |
| −4:29D | 29 | 343-371 |
| −4:32D | 32 | 343-374 |
| −4:34D | 34 | 343-376 |
| −4:35D | 35 | 343-377 |
| −4:38D | 38 | 343-380 |
| −4:44D | 44 | 343-386 |
| −4:5D | 5 | 343-347 |
| −4:7D | 7 | 343-349 |
| −4:8D | 8 | 343-350 |
| 4:9D | 9 | 343-351 |
| −57:66D | 66 | 290-355 |
| −5:11D | 11 | 342-352 |
| −5:12D | 12 | 342-353 |
| −5:13D | 13 | 342-354 |
| −5:14D | 14 | 342-355 |
| −5:16D | 16 | 342-357 |
| −5:17D | 17 | 342-358 |
| −5:18D | 18 | 342-359 |
| −5:19D | 19 | 342-360 |
| −5:20D | 20 | 342-361 |
| −5:21D | 21 | 342-362 |
| −5:22D | 22 | 342-363 |
| −5:24D | 24 | 342-365 |
| −5:25D | 25 | 342-366 |
| −5:26D | 26 | 342-367 |
| −5:27D | 27 | 342-368 |
| −5:28D | 28 | 342-369 |
| −5:31D | 31 | 342-372 |
| −5:32D | 32 | 342-373 |
| −5:34D | 34 | 342-375 |
| −5:35D | 35 | 342-376 |
| −5:3D | 3 | 342-344 |
| −5:40D | 40 | 342-381 |
| −5:41D | 41 | 342-382 |
| −5:6D | 6 | 342-347 |
| −5:7D | 7 | 342-348 |
| −5:9D | 9 | 342-350 |
| −5:9D | 9 | 342-350 |
| 8:22D | 22 | 354-375 |
| 35:5D | 5 | 381-385 |
| −64:69D | 69 | 283-351 |
| −66:70D | 70 | 281-350 |
| −68:69D | 69 | 279-347 |
| −6:10D | 10 | 341-350 |
| −6:11D | 11 | 341-351 |
| −6:12D | 12 | 341-352 |
| −6:14D | 14 | 341-354 |
| −6:15D | 15 | 341-355 |
| −6:16D | 16 | 341-356 |
| −6:17D | 17 | 341-357 |
| −6:21D | 21 | 341-361 |
| −6:22D | 22 | 341-362 |
| −6:23D | 23 | 341-363 |
| −6:24D | 24 | 341-364 |
| −6:25D | 25 | 341-365 |
| −6:26D | 26 | 341-366 |
| −6:33D | 33 | 341-373 |
| −6:40D | 40 | 341-380 |
| −6:5D | 5 | 341-345 |
| −6:7D | 7 | 341-347 |
| −6:8D | 8 | 341-348 |
| −6:9D | 9 | 341-349 |
| −71:73D | 73 | 276-348 |
| −7:11D | 11 | 340-350 |
| −7:13D | 13 | 340-352 |
| −7:14D | 14 | 340-353 |
| −7:15D | 15 | 340-354 |
| −7:16D | 16 | 340-355 |
| −7:18D | 18 | 340-357 |
| −7:19D | 19 | 340-358 |
| −7:1D | 1 | 340-340 |
| −7:20D | 20 | 340-359 |
| −7:21D | 21 | 340-360 |
| −7:22D | 22 | 340-361 |
| −7:23D | 23 | 340-362 |
| −7:24D | 24 | 340-363 |
| −7:26D | 26 | 340-365 |
| −7:27D | 27 | 340-366 |
| −7:28D | 28 | 340-367 |
| −7:29D | 29 | 340-368 |
| −7:30D | 30 | 340-369 |
| −7:31D | 31 | 340-370 |
| −7:33D | 33 | 340-372 |
| −7:34D | 34 | 340-373 |
| −7:36D | 36 | 340-375 |
| −7:37D | 37 | 340-376 |
| −7:40D | 40 | 340-379 |
| −7:41D | 41 | 340-380 |
| −7:44D | 44 | 340-383 |
| −7:45D | 45 | 340-384 |
| −7:47D | 47 | 340-386 |
| −7:4D | 4 | 340-343 |
| −7:8D | 8 | 340-347 |
| −7:9D | 9 | 340-348 |
| −8:10D | 10 | 339-348 |
| −8:12D | 12 | 339-350 |
| −8:14D | 14 | 339-352 |
| −8:15D | 15 | 339-353 |
| −8:16D | 16 | 339-354 |
| −8:17D | 17 | 339-355 |
| −8:19D | 19 | 339-357 |
| −8:20D | 20 | 339-358 |
| −8:22D | 22 | 339-360 |
| −8:23D | 23 | 339-361 |
| −8:24D | 24 | 339-362 |
| −8:25D | 25 | 339-363 |
| −8:27D | 27 | 339-365 |
| −8:28D | 28 | 339-366 |
| −8:29D | 29 | 339-367 |
| −8:30D | 30 | 339-368 |
| −8:31D | 31 | 339-369 |
| −8:32D | 32 | 339-370 |
| −8:34D | 34 | 339-372 |
| −8:35D | 35 | 339-373 |
| −8:37D | 37 | 339-375 |
| −8:38D | 38 | 339-376 |
| −8:39D | 39 | 339-377 |
| −8:41D | 41 | 339-379 |
| −8:44D | 44 | 339-382 |
| −8:7D | 7 | 339-345 |
| −8:9D | 9 | 339-347 |
| −9:11D | 11 | 338-348 |
| −9:12D | 12 | 338-349 |
| −9:14D | 14 | 338-351 |
| −9:16D | 16 | 338-353 |
| −9:18D | 18 | 338-355 |
| −9:19D | 19 | 338-356 |
| −9:20D | 20 | 338-357 |
| −9:21D | 21 | 338-358 |
| −9:22D | 22 | 338-359 |
| −9:23D | 23 | 338-360 |
| −9:24D | 24 | 338-361 |
| −9:25D | 25 | 338-362 |
| −9:26D | 26 | 338-363 |
| −9:27D | 27 | 338-364 |
| −9:28D | 28 | 338-365 |
| −9:29D | 29 | 338-366 |
| −9:31D | 31 | 338-368 |
| −9:32D | 32 | 338-369 |
| −9:33D | 33 | 338-370 |
| −9:35D | 35 | 338-372 |
| −9:36D | 36 | 338-373 |
| −9:38D | 38 | 338-375 |
| −9:40D | 40 | 338-377 |
| −9:42D | 42 | 338-379 |
| −9:8D | 8 | 338-345 |
| 1:11D | 11 | 347-357 |
| 1:12D | 12 | 347-358 |
| 1:15D | 15 | 347-361 |
| 1:16D | 16 | 347-362 |
| 1:17D | 17 | 347-363 |
| 1:1D | 1 | 347-347 |
| 1:23D | 23 | 347-369 |
| 1:2D | 2 | 347-348 |
| 1:33D | 33 | 347-379 |
| 1:34D | 34 | 347-380 |
| 1:39D | 39 | 347-385 |
| 1:42D | 42 | 347-388 |
| 1:4D | 4 | 347-350 |
| 1:6D | 6 | 347-352 |
| 1:7D | 7 | 347-353 |
| 1:8D | 8 | 347-354 |
| 1:9D | 9 | 347-355 |
| 2:10D | 10 | 348-357 |
| 2:11D | 11 | 348-358 |
| 2:13D | 13 | 348-360 |
| 2:15D | 15 | 348-362 |
| 2:16D | 16 | 348-363 |
| 2:18D | 18 | 348-365 |
| 2:1D | 1 | 348-348 |
| 2:25D | 25 | 348-372 |
| 2:28D | 28 | 348-375 |
| 2:2D | 2 | 348-349 |
| 2:34D | 34 | 348-381 |
| 2:4D | 4 | 348-351 |
| 2:6D | 6 | 348-353 |
| 2:8D | 8 | 348-355 |
| 3:1D | 1 | 349-349 |
| 3:2D | 2 | 349-350 |
| 3:3D | 3 | 349-351 |
| 3:4D | 4 | 349-352 |
| 3:5D | 5 | 349-353 |
| 4:13D | 13 | 350-362 |
| 4:17D | 17 | 350-366 |
| 4:1D | 1 | 350-350 |
| 5:1D | 1 | 351-351 |

TABLE 3
Additional Deletions in the JAG1 gene

| | | Deleted Nucleotides in the |
|---|---|---|
| | Deletion Size | corresponding JAG1 gene SEQ |
| Variant ID | (nucleotides) | ID NO: 18 reference sequence |
| 1:6D | 6 | 426-431 |
| −9:13D | 13 | 417-429 |
| −10:12D | 12 | 416-427 |
| −10:13D | 13 | 416-428 |
| −10:15D | 15 | 416-430 |
| −10:17D | 17 | 416-432 |
| −10:18D | 18 | 416-433 |
| −10:19D | 19 | 416-434 |
| −10:20D | 20 | 416-435 |
| −10:21D | 21 | 416-436 |
| −10:22D | 22 | 416-437 |
| −10:25D | 25 | 416-440 |
| −10:27D | 27 | 416-442 |
| −10:28D | 28 | 416-443 |
| −10:29D | 29 | 416-444 |
| −10:34D | 34 | 416-449 |
| −10:35D | 35 | 416-450 |
| −10:37D | 37 | 416-452 |
| −10:39D | 39 | 416-454 |
| −10:48D | 48 | 416-463 |
| −10:55D | 55 | 416-470 |
| −10:65D | 65 | 416-480 |
| −11:13D | 13 | 415-427 |
| −11:14D | 14 | 415-428 |
| −11:16D | 16 | 415-430 |
| −11:18D | 18 | 415-432 |
| −11:19D | 19 | 415-433 |
| −11:20D | 20 | 415-434 |
| −11:21D | 21 | 415-435 |
| −11:22D | 22 | 415-436 |
| −11:24D | 24 | 415-438 |
| −11:30D | 30 | 415-444 |
| −11:31D | 31 | 415-445 |
| −11:46D | 46 | 415-460 |
| −11:49D | 49 | 415-463 |
| −11:52D | 52 | 415-466 |
| −11:54D | 54 | 415-468 |
| −11:58D | 58 | 415-472 |
| −11:60D | 60 | 415-474 |
| −12:13D | 13 | 414-426 |
| −12:16D | 16 | 414-429 |
| −12:17D | 17 | 414-430 |
| −12:18D | 18 | 414-431 |
| −12:33D | 33 | 414-446 |
| −12:34D | 34 | 414-447 |
| −12:51D | 51 | 414-464 |
| −12:53D | 53 | 414-466 |
| −13:15D | 15 | 413-427 |
| −13:16D | 16 | 413-428 |
| −13:18D | 18 | 413-430 |
| −13:20D | 20 | 413-432 |
| −13:21D | 21 | 413-433 |
| −13:22D | 22 | 413-434 |
| −13:23D | 23 | 413-435 |
| −13:24D | 24 | 413-436 |
| −13:25D | 25 | 413-437 |
| −13:29D | 29 | 413-441 |
| −13:39D | 39 | 413-451 |
| −13:50D | 50 | 413-462 |
| −13:9D | 9 | 413-421 |
| −14:17D | 17 | 412-428 |
| −14:19D | 19 | 412-430 |
| −14:22D | 22 | 412-433 |
| −14:23D | 23 | 412-434 |
| −14:24D | 24 | 412-435 |
| −14:27D | 27 | 412-438 |
| −14:29D | 29 | 412-440 |
| −14:44D | 44 | 412-455 |
| −14:55D | 55 | 412-466 |
| −15:18D | 18 | 411-428 |
| −15:20D | 20 | 411-430 |
| −15:22D | 22 | 411-432 |
| −15:23D | 23 | 411-433 |
| −15:24D | 24 | 411-434 |
| −15:25D | 25 | 411-435 |
| −15:26D | 26 | 411-436 |
| −15:27D | 27 | 411-437 |
| −15:28D | 28 | 411-438 |
| −15:34D | 34 | 411-444 |
| −15:53D | 53 | 411-463 |
| −15:56D | 56 | 411-466 |
| −15:60D | 60 | 411-470 |
| −16:20D | 20 | 410-429 |
| −16:28D | 28 | 410-437 |
| −16:35D | 35 | 410-444 |
| −16:36D | 36 | 410-445 |
| −16:37D | 37 | 410-446 |
| −16:57D | 57 | 410-466 |
| −16:59D | 59 | 410-468 |
| −16:65D | 65 | 410-474 |
| −17:19D | 19 | 409-427 |
| −17:20D | 20 | 409-428 |
| −17:21D | 21 | 409-429 |
| −17:23D | 23 | 409-431 |
| −17:24D | 24 | 409-432 |
| −17:29D | 29 | 409-437 |
| −17:43D | 43 | 409-451 |
| −17:48D | 48 | 409-456 |
| −17:49D | 49 | 409-457 |
| −17:54D | 54 | 409-462 |
| −18:19D | 19 | 408-426 |
| −18:22D | 22 | 408-429 |
| −18:23D | 23 | 408-430 |
| −18:28D | 28 | 408-435 |
| −18:29D | 29 | 408-436 |
| −18:55D | 55 | 408-462 |
| −19:18D | 18 | 407-424 |
| −19:22D | 22 | 407-428 |
| −19:26D | 26 | 407-432 |
| −19:36D | 36 | 407-442 |
| −1:10D | 10 | 425-434 |
| −1:11D | 11 | 425-435 |
| −1:12D | 12 | 425-436 |
| −1:13D | 13 | 425-437 |
| −1:14D | 14 | 425-438 |
| −1:16D | 16 | 425-440 |
| −1:17D | 17 | 425-441 |
| −1:18D | 18 | 425-442 |
| −1:19D | 19 | 425-443 |
| −1:20D | 20 | 425-444 |
| −1:25D | 25 | 425-449 |
| −1:28D | 28 | 425-452 |
| −1:30D | 30 | 425-454 |
| −1:33D | 33 | 425-457 |
| −1:34D | 34 | 425-458 |
| −1:3D | 3 | 425-427 |
| −1:48D | 48 | 425-472 |
| −1:4D | 4 | 425-428 |
| −1:52D | 52 | 425-476 |
| −1:54D | 54 | 425-478 |
| −1:55D | 55 | 425-479 |
| −1:58D | 58 | 425-482 |
| −1:6D | 6 | 425-430 |
| −1:8D | 8 | 425-432 |
| −1:9D | 9 | 425-433 |
| −20:36D | 36 | 406-441 |
| −20:53D | 53 | 406-458 |
| −21:24D | 24 | 405-428 |
| −21:25D | 25 | 405-429 |
| −21:26D | 26 | 405-430 |
| −21:28D | 28 | 405-432 |
| −21:48D | 48 | 405-452 |
| −21:54D | 54 | 405-458 |
| −21:68D | 68 | 405-472 |
| −21:70D | 70 | 405-474 |
| −22:30D | 30 | 404-433 |
| −23:26D | 26 | 403-428 |
| −23:32D | 32 | 403-434 |
| −23:36D | 36 | 403-438 |
| −23:37D | 37 | 403-439 |
| −23:66D | 66 | 403-468 |
| −23:72D | 72 | 403-474 |
| −24:31D | 31 | 402-432 |
| −24:33D | 33 | 402-434 |
| −24:36D | 36 | 402-437 |
| −24:39D | 39 | 402-440 |
| −24:41D | 41 | 402-442 |
| −25:32D | 32 | 401-432 |
| −25:35D | 35 | 401-435 |
| −25:38D | 38 | 401-438 |
| −25:44D | 44 | 401-444 |
| −25:52D | 52 | 401-452 |
| −25:58D | 58 | 401-458 |
| −26:30D | 30 | 400-429 |
| −26:45D | 45 | 400-444 |
| −26:68D | 68 | 400-467 |
| −26:72D | 72 | 400-471 |
| −27:50D | 50 | 399-448 |
| −28:47D | 47 | 398-444 |
| −29:31D | 31 | 397-427 |
| −29:36D | 36 | 397-432 |
| −29:37D | 37 | 397-433 |
| −29:41D | 41 | 397-437 |
| −2:10D | 10 | 424-433 |
| −2:11D | 11 | 424-434 |
| −2:12D | 12 | 424-435 |
| −2:13D | 13 | 424-436 |
| −2:14D | 14 | 424-437 |
| −2:15D | 15 | 424-438 |
| −2:17D | 17 | 424-440 |
| −2:18D | 18 | 424-441 |
| −2:19D | 19 | 424-442 |
| −2:20D | 20 | 424-443 |
| −2:21D | 21 | 424-444 |
| −2:22D | 22 | 424-445 |
| −2:27D | 27 | 424-450 |
| −2:29D | 29 | 424-452 |
| −2:31D | 31 | 424-454 |
| −2:32D | 32 | 424-455 |
| −2:34D | 34 | 424-457 |
| −2:35D | 35 | 424-458 |
| −2:37D | 37 | 424-460 |
| −2:40D | 40 | 424-463 |
| −2:45D | 45 | 424-468 |
| −2:47D | 47 | 424-470 |
| −2:49D | 49 | 424-472 |
| −2:4D | 4 | 424-427 |
| −2:51D | 51 | 424-474 |
| −2:57D | 57 | 424-480 |
| −2:5D | 5 | 424-428 |
| −2:60D | 60 | 424-483 |
| −2:61D | 61 | 424-484 |
| −2:7D | 7 | 424-430 |
| −2:9D | 9 | 424-432 |
| −30:33D | 33 | 396-428 |
| −30:36D | 36 | 396-431 |
| −30:37D | 37 | 396-432 |
| −31:35D | 35 | 395-429 |
| −31:36D | 36 | 395-430 |
| −31:38D | 38 | 395-432 |
| −31:39D | 39 | 395-433 |
| −31:41D | 41 | 395-435 |
| −31:65D | 65 | 395-459 |
| −32:35D | 35 | 394-428 |
| −32:47D | 47 | 394-440 |
| −32:50D | 50 | 394-443 |
| −32:62D | 62 | 394-455 |
| −32:64D | 64 | 394-457 |
| −33:36D | 36 | 393-428 |
| −33:53D | 53 | 393-445 |
| −33:55D | 55 | 393-447 |
| −35:43D | 43 | 391-433 |
| −35:46D | 46 | 391-436 |
| −35:48D | 48 | 391-438 |
| −36:40D | 40 | 390-429 |
| −36:63D | 63 | 390-452 |
| −37:38D | 38 | 389-426 |
| −38:39D | 39 | 388-426 |
| −38:42D | 42 | 388-429 |
| −38:61D | 61 | 388-448 |
| −39:40D | 40 | 387-426 |
| −39:54D | 54 | 387-440 |
| −39:61D | 61 | 387-447 |
| −3:10D | 10 | 423-432 |
| −3:11D | 11 | 423-433 |
| −3:12D | 12 | 423-434 |
| −3:13D | 13 | 423-435 |
| −3:14D | 14 | 423-436 |
| −3:15D | 15 | 423-437 |
| −3:16D | 16 | 423-438 |
| −3:17D | 17 | 423-439 |
| −3:18D | 18 | 423-440 |
| −3:19D | 19 | 423-441 |
| −3:1D | 1 | 423-423 |
| −3:20D | 20 | 423-442 |
| −3:23D | 23 | 423-445 |
| −3:24D | 24 | 423-446 |
| −3:25D | 25 | 423-447 |
| −3:26D | 26 | 423-448 |
| −3:27D | 27 | 423-449 |
| −3:28D | 28 | 423-450 |
| −3:29D | 29 | 423-451 |
| −3:2D | 2 | 423-424 |
| −3:30D | 30 | 423-452 |
| −3:31D | 31 | 423-453 |
| −3:37D | 37 | 423-459 |
| −3:38D | 38 | 423-460 |
| −3:39D | 39 | 423-461 |
| −3:40D | 40 | 423-462 |
| −3:41D | 41 | 423-463 |
| −3:42D | 42 | 423-464 |
| −3:43D | 43 | 423-465 |
| −3:44D | 44 | 423-466 |
| −3:46D | 46 | 423-468 |
| −3:48D | 48 | 423-470 |
| −3:49D | 49 | 423-471 |
| −3:4D | 4 | 423-426 |
| −3:50D | 50 | 423-472 |
| −3:51D | 51 | 423-473 |
| −3:52D | 52 | 423-474 |
| −3:53D | 53 | 423-475 |
| −3:54D | 54 | 423-476 |
| −3:55D | 55 | 423-477 |
| −3:58D | 58 | 423-480 |
| −3:5D | 5 | 423-427 |
| −3:60D | 60 | 423-482 |
| −3:6D | 6 | 423-428 |
| −3:7D | 7 | 423-429 |
| −3:8D | 8 | 423-430 |
| −3:9D | 9 | 423-431 |
| −40:45D | 45 | 386-430 |
| −40:46D | 46 | 386-431 |
| −40:54D | 54 | 386-439 |
| −40:55D | 55 | 386-440 |
| −41:45D | 45 | 385-429 |
| −41:48D | 48 | 385-432 |
| −41:55D | 55 | 385-439 |
| −42:46D | 46 | 384-429 |
| −42:56D | 56 | 384-439 |
| −43:51D | 51 | 383-433 |
| −43:53D | 53 | 383-435 |
| −44:52D | 52 | 382-433 |
| −44:53D | 53 | 382-434 |
| −44:54D | 54 | 382-435 |
| −44:55D | 55 | 382-436 |
| −44:60D | 60 | 382-441 |
| −45:48D | 48 | 381-428 |
| −45:58D | 58 | 381-438 |
| −47:50D | 50 | 379-428 |
| −4:10D | 10 | 422-431 |
| −4:13D | 13 | 422-434 |
| −4:15D | 15 | 422-436 |
| −4:16D | 16 | 422-437 |
| −4:18D | 18 | 422-439 |
| −4:19D | 19 | 422-440 |
| −4:22D | 22 | 422-443 |
| −4:23D | 23 | 422-444 |
| −4:24D | 24 | 422-445 |
| −4:25D | 25 | 422-446 |
| −4:26D | 26 | 422-447 |
| −4:29D | 29 | 422-450 |
| −4:2D | 2 | 422-423 |
| −4:30D | 30 | 422-451 |
| −4:33D | 33 | 422-454 |
| −4:34D | 34 | 422-455 |
| −4:36D | 36 | 422-457 |
| −4:39D | 39 | 422-460 |
| −4:3D | 3 | 422-424 |
| −4:44D | 44 | 422-465 |
| −4:49D | 49 | 422-470 |
| −4:50D | 50 | 422-471 |
| −4:51D | 51 | 422-472 |
| −4:52D | 52 | 422-473 |
| −4:53D | 53 | 422-474 |
| −4:54D | 54 | 422-475 |
| −4:55D | 55 | 422-476 |
| −4:56D | 56 | 422-477 |
| −4:57D | 57 | 422-478 |
| −4:5D | 5 | 422-426 |
| −4:62D | 62 | 422-483 |
| −4:8D | 8 | 422-429 |
| −4:9D | 9 | 422-430 |
| −50:54D | 54 | 376-429 |
| −5:11D | 11 | 421-431 |
| −5:12D | 12 | 421-432 |
| −5:13D | 13 | 421-433 |
| −5:15D | 15 | 421-435 |
| −5:18D | 18 | 421-438 |
| −5:19D | 19 | 421-439 |
| −5:21D | 21 | 421-441 |
| −5:23D | 23 | 421-443 |
| −5:26D | 26 | 421-446 |
| −5:28D | 28 | 421-448 |
| −5:32D | 32 | 421-452 |
| −5:38D | 38 | 421-458 |
| −5:42D | 42 | 421-462 |
| −5:43D | 43 | 421-463 |
| −5:44D | 44 | 421-464 |
| −5:49D | 49 | 421-469 |
| −5:51D | 51 | 421-471 |
| −5:57D | 57 | 421-477 |
| −5:59D | 59 | 421-479 |
| −5:61D | 61 | 421-481 |
| −5:62D | 62 | 421-482 |
| −5:6D | 6 | 421-426 |
| −5:7D | 7 | 421-427 |
| −5:8D | 8 | 421-428 |
| −5:9D | 9 | 421-429 |
| −6:10D | 10 | 420-429 |
| −6:11D | 11 | 420-430 |
| −6:12D | 12 | 420-431 |
| −6:15D | 15 | 420-434 |
| −6:17D | 17 | 420-436 |
| −6:18D | 18 | 420-437 |
| −6:20D | 20 | 420-439 |
| −6:25D | 25 | 420-444 |
| −6:26D | 26 | 420-445 |
| −6:31D | 31 | 420-450 |
| −6:35D | 35 | 420-454 |
| −6:37D | 37 | 420-456 |
| −6:3D | 3 | 420-422 |
| −6:40D | 40 | 420-459 |
| −6:50D | 50 | 420-469 |
| −6:52D | 52 | 420-471 |
| −6:54D | 54 | 420-473 |
| −6:55D | 55 | 420-474 |
| −6:56D | 56 | 420-475 |
| −6:58D | 58 | 420-477 |
| −6:64D | 64 | 420-483 |
| −6:7D | 7 | 420-426 |
| −7:11D | 11 | 419-429 |
| −7:12D | 12 | 419-430 |
| −7:13D | 13 | 419-431 |
| −7:16D | 16 | 419-434 |
| −7:18D | 18 | 419-436 |
| −7:19D | 19 | 419-437 |
| −7:22D | 22 | 419-440 |
| −7:28D | 28 | 419-446 |
| −7:29D | 29 | 419-447 |
| −7:37D | 37 | 419-455 |
| −7:39D | 39 | 419-457 |
| −7:41D | 41 | 419-459 |
| −7:47D | 47 | 419-465 |
| −7:50D | 50 | 419-468 |
| −7:52D | 52 | 419-470 |
| −7:55D | 55 | 419-473 |
| −7:58D | 58 | 419-476 |
| −7:6D | 6 | 419-424 |
| −7:8D | 8 | 419-426 |
| −8:10D | 10 | 418-427 |
| −8:11D | 11 | 418-428 |
| −8:12D | 12 | 418-429 |
| −8:14D | 14 | 418-431 |
| −8:15D | 15 | 418-432 |
| −8:16D | 16 | 418-433 |
| −8:18D | 18 | 418-435 |
| −8:21D | 21 | 418-438 |
| −8:22D | 22 | 418-439 |
| −8:24D | 24 | 418-441 |
| −8:32D | 32 | 418-449 |
| −8:35D | 35 | 418-452 |
| −8:36D | 36 | 418-453 |
| −8:44D | 44 | 418-461 |
| −8:45D | 45 | 418-462 |
| −8:47D | 47 | 418-464 |
| −8:56D | 56 | 418-473 |
| −8:60D | 60 | 418-477 |
| −8:62D | 62 | 418-479 |
| −8:65D | 65 | 418-482 |
| −8:9D | 9 | 418-426 |
| −9:10D | 10 | 417-426 |
| −9:11D | 11 | 417-427 |
| −9:12D | 12 | 417-428 |
| −9:13D | 13 | 417-429 |
| −9:14D | 14 | 417-430 |
| −9:15D | 15 | 417-431 |
| −9:16D | 16 | 417-432 |
| −9:17D | 17 | 417-433 |
| −9:19D | 19 | 417-435 |
| −9:20D | 20 | 417-436 |
| −9:23D | 23 | 417-439 |
| −9:33D | 33 | 417-449 |
| −9:34D | 34 | 417-450 |
| −9:37D | 37 | 417-453 |
| −9:43D | 43 | 417-459 |
| −9:49D | 49 | 417-465 |
| −9:51D | 51 | 417-467 |
| −9:53D | 53 | 417-469 |
| −9:55D | 55 | 417-471 |
| −9:57D | 57 | 417-473 |
| 1:10D | 10 | 426-435 |
| 1:11D | 11 | 426-436 |
| 1:12D | 12 | 426-437 |
| 1:13D | 13 | 426-438 |
| 1:14D | 14 | 426-439 |
| 1:15D | 15 | 426-440 |
| 1:16D | 16 | 426-441 |
| 1:17D | 17 | 426-442 |
| 1:1D | 1 | 426-426 |
| 1:20D | 20 | 426-445 |
| 1:21D | 21 | 426-446 |
| 1:22D | 22 | 426-447 |
| 1:23D | 23 | 426-448 |
| 1:25D | 25 | 426-450 |
| 1:27D | 27 | 426-452 |
| 1:2D | 2 | 426-427 |
| 1:33D | 33 | 426-458 |
| 1:35D | 35 | 426-460 |
| 1:3D | 3 | 426-428 |
| 1:40D | 40 | 426-465 |
| 1:43D | 43 | 426-468 |
| 1:44D | 44 | 426-469 |
| 1:46D | 46 | 426-471 |
| 1:47D | 47 | 426-472 |
| 1:4D | 4 | 426-429 |
| 1:50D | 50 | 426-475 |
| 1:57D | 57 | 426-482 |
| 1:5D | 5 | 426-430 |
| 1:6D | 6 | 426-431 |
| 1:7D | 7 | 426-432 |
| 1:8D | 8 | 426-433 |
| 1:9D | 9 | 426-434 |
| 2:12D | 12 | 427-438 |
| 2:14D | 14 | 427-440 |
| 2:15D | 15 | 427-441 |
| 2:1D | 1 | 427-427 |
| 2:2D | 2 | 427-428 |
| 2:31D | 31 | 427-457 |
| 2:37D | 37 | 427-463 |
| 2:4D | 4 | 427-430 |
| 2:6D | 6 | 427-432 |
| 2:7D | 7 | 427-433 |
| 2:8D | 8 | 427-434 |
| 3:13D | 13 | 428-440 |
| 3:18D | 18 | 428-445 |
| 3:2D | 2 | 428-429 |
| 3:30D | 30 | 428-457 |
| 3:3D | 3 | 428-430 |
| 3:43D | 43 | 428-470 |
| 3:49D | 49 | 428-476 |
| 3:4D | 4 | 428-431 |
| 3:56D | 56 | 428-483 |
| 3:9D | 9 | 428-436 |
| 4:1D | 1 | 429-429 |
| 4:2D | 2 | 429-430 |
| 4:6D | 6 | 429-434 |
| 5:1D | 1 | 430-430 |
| 6:19D | 19 | 431-449 |
| 6:1D | 1 | 431-431 |
| 6:3D | 3 | 431-433 |
| 6:5D | 5 | 431-435 |

### Example 5. Performance of Additional Soybeans with Mutated FT1a and JAG1. Genes in Field Tests

Field tests on the SENF2225 and SENF2226 soybean plants were performed at various locations in the midwestern United States during two growing seasons as described in Tables 4-11. The second growing season results are summarized in Table 12. The check (control lacking the FT1a and JAG1 mutations) is NINF1170.

TABLE 4
Shadeland, IN Growing Season 1
Year: Growing Season 1
Location: Shadeland, IN
Replicates within location: 3
Type of experiment: Single-row (6 ft. long)

| | SENF2225 | SENF2226 | NINF1170 |
|---|---|---|---|
| Seed Yield ¹ | 64.3 ± 11.4 | 46.2 ± 23.1 | 57.4 ± 6 |
| Plants Per | 102703 ± 5529 | 122181 ± 7028 | 91193 ± 14629 |
| Acre ² | | | |
| Plant Height ³ | Not collected | Not collected | Not collected |
| Time To | 45 ± 1 | 47 ± 1 | 46 ± 2 |
| Flowering ⁴ | | | |
| Time to | 67 ± 2 | 65 ± 0 | 67 ± 2 |
| Beginning Pod | | | |
| Time to | 80 ± 2 | 81 ± 0 | 81 ± 0 |
| Beginning | | | |
| Seed ⁶ | | | |
| Time to Full | 119 ± 1 | 119 ± 1 | 120±1 |
| Maturity ⁷ | | | |

TABLE 5
Cumberland, IA Growing Season 2
Year: Growing Season 2
Location: Cumberland, IA
Replicates within location: 3
Type of experiment: Yield trial (Plots of
4 20 ft. Rows; 2 central rows evaluated)

| | SENF2225 | SENF2226 | NINF1170 |
|---|---|---|---|
| Seed Yield ¹ | 62.3 ± 1.2 | 65.3 ± 3.1 | 60.1 ± 6.2 |
| Plants Per Acre ² | 74371 ± 7512 | 67288 ± 9328 | 64632 ± 10735 |
| Plant Height ³ | 37.5 ± 0.7 | 38 ± 1 | 38.3 ± 0.6 |
| Time To | 56 ± 0 | 55 ± 1 | 54 ± 2 |
| Flowering ⁴ | | | |
| Time to | 75 ± 1 | 75 ± 1 | 76 ± 1 |
| Beginning Pod | | | |
| Time to | 92 ± 1 | 92 ± 2 | 94 ± 1 |
| Beginning Seed ⁶ | | | |
| Time to Full | 124 ± 0 | 124 ± 0 | 127 ± 3 |
| Maturity ⁷ | | | |

TABLE 6
Walnut, IL Growing Season 2
Year: Growing Season 2
Location: Walnut, IL
Replicates within location: 3
Type of experiment: Yield trial (Plots of
4 20 ft. Rows; 2 central rows evaluated)

| | SENF2225 | SENF2226 | NINF1170 |
|---|---|---|---|
| Seed Yield ¹ | 64.4 ± 10.7 | 69.7 ± 3.2 | 57.1 ± 5.1 |
| Plants Per Acre ² | 71715 ± 4601 | 73486 ± 7667 | 56663 ± 8538 |
| Plant Height ³ | 31.7 ± 0.6 | 34.3 ± 2.1 | 34.7 ± 2.1 |
| Time To Flowering ⁴ | 53 ± 0 | 53 ± 0 | 53 ± 0 |
| Time to | 69 ± 1 | 68 ± 1 | 68 ± 0 |
| Beginning Pod | | | |
| Time to | 83 ± 1 | 83 ± 1 | 82 ± 0 |
| Beginning Seed ⁶ | | | |
| Time to Full | 130 ± 2 | 132 ± 1 | 132 ± 1 |
| Maturity ⁷ | | | |

TABLE 7
Aurora, NE Growing Season 2
Year: Growing Season 2
Location: Aurora, NE
Replicates within location: 3
Type of experiment: Yield trial (Plots of
4 20 ft. Rows; 2 central rows evaluated)

| | SENF2225 | SENF2226 | NINF1170 |
|---|---|---|---|
| Seed Yield ¹ | 66.8 ± 3.8 | 71.8 ± 7.4 | 62.4 ± 4.2 |
| Plants Per | 107129 ± 19215 | 118197 ± 9391.1 | 101817 ± 9328 |
| Acre ² | | | |
| Plant Height ³ | 30.7 ± 1.2 | 32.5 ± 1 | 31 ± 0 |
| Time To | Not collected | Not collected | Not collected |
| Flowering ⁴ | | | |
| Time to | Not collected | Not collected | Not collected |
| Beginning Pod | | | |
| Time to | Not collected | Not collected | Not collected |
| Beginning | | | |
| Seed ⁶ | | | |
| Time to Full | Not collected | Not collected | Not collected |
| Maturity ⁷ | | | |

TABLE 8
Shadeland, IN Growing Season 2
Year: Growing Season 2
Location: Shadeland, IN
Replicates within location: 3
Type of experiment: Yield trial (Plots of
4 20 ft. Rows; 2 central rows evaluated)

| | SENF2225 | SENF2226 | NINF1170 |
|---|---|---|---|
| Seed Yield ¹ | 69.7 ± 3.7 | 68.7 ± 1.2 | 68.7 ± 2.8 |
| Plants Per | 102703 ± 5529 | 122181 ± 7028 | 91193 ± 14629 |
| Acre ² | | | |
| Plant Height ³ | 36.3 ± 5.9 | 35.3 ± 0.6 | 34.3 ± 1.5 |
| Time To | 47 ± 2 | 48 ± 1 | 47 ± 1 |
| Flowering ⁴ | | | |
| Time to | 64 ± 1 | 66 ± 2 | 65 ± 2 |
| Beginning Pod | | | |
| Time to | 83 ± 2 | 84 ± 1 | 82 ± 1 |
| Beginning | | | |
| Seed ⁶ | | | |
| Time to Full | 125 ± 2 | 125 ± 2 | 126 ± 1 |
| Maturity ⁷ | | | |

TABLE 9
Jefferson, IA Growing Season 2
Year: Growing Season 2
Location: Jefferson, IA
Replicates within location: 3
Type of experiment: Yield trial (Plots of
4 20 ft. Rows; 2 central rows evaluated)

| | SENF2225 | SENF2226 | NINF1170 |
|---|---|---|---|
| Seed Yield ¹ | 63.1 ± 2.7 | 56.8 ± 6.8 | 62.2 ± 6.7 |
| Plants Per | 130149 ± 14788 | 124837 ± 2657 | 108900 ± 11577 |
| Acre ² | | | |
| Plant Height ³ | 33 ± 2.6 | 35 ± 1 | 34 ± 3.6 |
| Time To | 53 ± 1 | 53 ± 1 | 52 ± 0 |
| Flowering ⁴ | | | |
| Time to | 67 ± 0 | 67 ± 0 | 67 ± 1 |
| Beginning Pod | | | |
| Time to | 87 ± 0 | 87 ± 0 | 87 ± 2 |
| Beginning | | | |
| Seed ⁶ | | | |
| Time to Full | 130 ± 1 | 123 ± 12 | 133 ± 4 |
| Maturity ⁷ | | | |

TABLE 10
Kimbalton, IA Growing Season 2
Year: Growing Season 2
Location: Kimbalton, IA
Replicates within location: 3
Type of experiment: Yield trial (Plots of
4 20 ft. Rows; 2 central rows evaluated)

| | SENF2225 | SENF2226 | NINF1170 |
|---|---|---|---|
| Seed Yield ¹ | 70.9 ± 2.2 | 71.8 ± 0.5 | 63 ± 0.9 |
| Plants Per | 108900 ± 9576 | 132805 ± 7027 | 115983 ± 3067 |
| Acre ² | | | |
| Plant Height ³ | 35 ± 2 | 35.7 ± 2.1 | 36.3 ± 2.9 |
| Time To | 50.3 ± 0.6 | 52 ± 0 | 52 ± 0 |
| Flowering ⁴ | | | |
| Time to | 64.3 ± 1.2 | 65 ± 0 | 65.7 ± 1.2 |
| Beginning Pod | | | |
| Time to | 89.3 ± 1.2 | 90 ± 0 | 90.7 ± 1.2 |
| Beginning | | | |
| Seed ⁶ | | | |
| Time to Full | 126 ± 1.7 | 125.3 ± 0.6 | 126.7 ± 1.2 |
| Maturity ⁷ | | | |

TABLE 11
Elkart, IN Growing Season 2
Year: Growing Season 2
Location: Elkart, IN
Replicates within location: 3
Type of experiment: Yield trial (Plots of
4 20 ft. Rows; 2 central rows evaluated)

| | SENF2225 | SENF2226 | NINF1170 |
|---|---|---|---|
| Seed Yield ¹ | 65.7 ± 0.6 | 62 ± 6.4 | 59.6 ± 0.8 |
| Plants Per Acre ² | 140773 ± 0 | 140773 ± 0 | 140773 ± 0 |
| Plant Height ³ | Not collected | Not collected | Not collected |
| Time To Flowering ⁴ | Not collected | Not collected | Not collected |
| Time to | Not collected | Not collected | Not collected |
| Beginning Pod | | | |
| Time to | Not collected | Not collected | Not collected |
| Beginning Seed ⁶ | | | |
| Time to Full | Not collected | Not collected | Not collected |
| Maturity ⁷ | | | |

TABLE 12
Summary of Growing Season 2 field tests across all locations
Year: Growing Season 2
Location: All 7 locations
Replicates within location: 3
Type of experiment: Yield trial (Plots of
4 20 ft. Rows; 2 central rows evaluated)

| | SENF2225 | SENF2226 | NINF1170 |
|---|---|---|---|
| Seed Yield ¹ | 66.3 ± 5.1 | 66.3 ± 6.5 | 62 ± 7 |
| Plants Per | 106642 ± 25626 | 111025 ± 28731 | 98497 ± 26112 |
| Acre ² | | | |
| Plant Height ³ | 33.8 ± 3.5 | 35.3 ± 2 | 34.1 ± 3.8 |
| Time To | 53.6 ± 5.7 | 53.4 ± 4.6 | 51.3 ± 5.5 |
| Flowering ⁴ | | | |
| Time to | 69.4 ± 5.7 | 69.5 ± 5 | 66.5 ± 7.2 |
| Beginning Pod | | | |
| Time to | 87.7 ± 4.1 | 87.9 ± 4.1 | 86.1 ± 5.6 |
| Beginning | | | |
| Seed ⁶ | | | |
| Seed Yield ¹ | 130.2 ± 7.1 | 128.1 ± 7.9 | 127.4 ± 8.7 |

¹ “Seed Yield” refers to the yield adjusted to 12% moisture in bushels/acre of the grain at harvest.
² “Plants Per Acre” refers to the estimated number of soybean plants per acre.
³ “Plant height” refers to the plant height taken from soil level to the apical node on the main stem of the plant at maturity and is measured in inches.
⁴ “Time to Flowering” refers to the number of days from planting when 50% of the plants have at least one open flower at any node on the main stem
⁵ “Time to Beginning Pod” refers to the number of days from planting when 50% of plants had pods 3/16 inch long at one of the four uppermost nodes on the main stem with a fully developed leaf.
⁶ “Time to Beginning Seed” refers to the number of days from planting when 50% of the plants have seed ⅛ inch long in a pod at one of the four uppermost nodes on the main stem with a fully developed leaf.
⁷ “Time to Full Maturity” refers to the number of days from planting when 50% of the plants had 95% of their pods reach full maturity color.

### Example 6. Generation of mBS1 or mBS2 Soybean

The plasmid pIN1512 was created to transform soybean plants and disrupt the open reading frame of the of GmBS1 (Glyma10g38970; SEQ ID NO: 25) and/or GmBS2 (Glyma20g28840; SEQ ID NO: 26) genes through CRISPR-mediated gene editing. This plasmid was constructed using the strategy and techniques described by Čtermák et al., 2017, The Plant Cell. 29 (6) 1196-1217; DOI: 10.1105/tpc.16.00922). The pIN1512vector has the following two functional expression cassettes between the right and left T-DNA border. A dicot ubiquitin gene promoter and 5′ untranslated region (UTR) drives the expression a CRISPR-Cas nuclease transcript. The Cas gene had a SV40 nuclear localization signal (NLS) fused to the 5′ end and a nucleoplasmin NLS fused to the 3′ end. The coding sequence was followed by an Arabidopsis thaliana heat shock gene terminator. Another expression cassette is made up of an Arabidopsis thaliana U6-26 promoter driving the expression of a CRISPR guide RNA comprising a crRNA fused to a spacer RNA (encoded by SEQ ID NO: 36) designed to target the Glycine max BS1 or BS2 gene and followed by an RNA polymerase iii termination signal.

The other functional elements of the pIN1512 vector were derived from a standard Agrobacterium binary transformation plasmid that can replicate in both Escherichia coli and Agrobacterium tumefaciens. They are a T-DNA right border sequence followed by an expression cassette to confer glyphosate resistance to the transgenic plants. This cassette consisted of the Arabidopsis thaliana Ubiquitin 10 gene promoter and 5′ UTR, Agrobacterium sp. strain CP4 5-enolpyruvylshikimate-3-phosphate synthase (EPSPS) gene and the Pisum sativum rib-1,5-bisphospate carboxylase (rbcS) small subunit gene terminator. The insertion site was followed by a T-DNA left border sequence.

The plasmid pIN1340 was transformed into Agrobacterium tumefaciens EHA105 (Hood et al., 1993, Transgenic Research. 2: 208-218. doi:10.1007/BF01977351) by electroporation following standard techniques. Frozen glycerol stocks were prepared for use in plant transformation.

Transgenic T0 soybean events were made by Agrobacterium-mediated transformation with vector pIN1512. Sterilized soybean seeds were imbibed in water overnight, and explants were prepared as mature cotyledon halves with trimmed hypocotyls. The explants went through the typical transformation and regeneration steps of infection and co-cultivation, shoot induction and elongation and selection, rooting, and transplanting to soil to produce T1 seeds (see, for example, Li et al, Optimization of Agrobacterium-Mediated Transformation in Soybean (2017) Frontiers in Plant Science v8 Article 246; Pareddy et al. Transgenic Res. 2020 June; 29(3):267-281. doi: 10.1007/s11248-020-00198-8).

T0 plants were grown and genotyped by amplicon sequencing (AmpSeq).

### Example 7. Performance of mBS1 or mBS2 Soybean

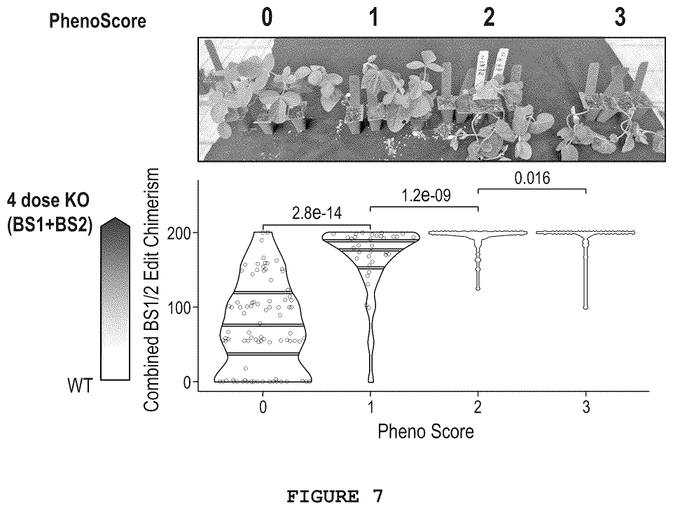

T0 regenerants with chimeric edited tissues were analyzed in the greenhouse. T0 plants comprising chimeric edited tissue containing: (i) no edits in the two BS1 or two BS2 genes (i.e., an editing dosage of 0 in the total of four BS1 and BS2 genes); (ii) an edit (e.g., deletion) in one of the two BS1 or in one of the two BS2 genes (i.e., an editing dosage of 1); (iii) an edit in two of the total of four BS1 and BS2 genes (i.e., an editing dosage of 2); (iv) an edit in of 3 of the total of four BS1 and BS2 genes (i.e., an editing dosage of 3); or (v) an edit in all four of the BS1 and BS2 genes (i.e., an editing dosage of 4) were analyzed. The editing dosage for BS1 and BS2 was correlated to a phenotypic score (“Pheno Score”) as shown in FIG. 7. A Pheno Score of 0 was given to soybean plants with a wild-type phenotype while a Pheno Score of 1, 2, or 3 was given to T0 regenerants with increasingly impaired growth where a score of 3 is the most impaired. A correlation between increased editing dosages of BS1 and BS2 and impaired growth was observed.

### Example 8. Analysis of T1 mBS1 and mBS2 Soybean Families

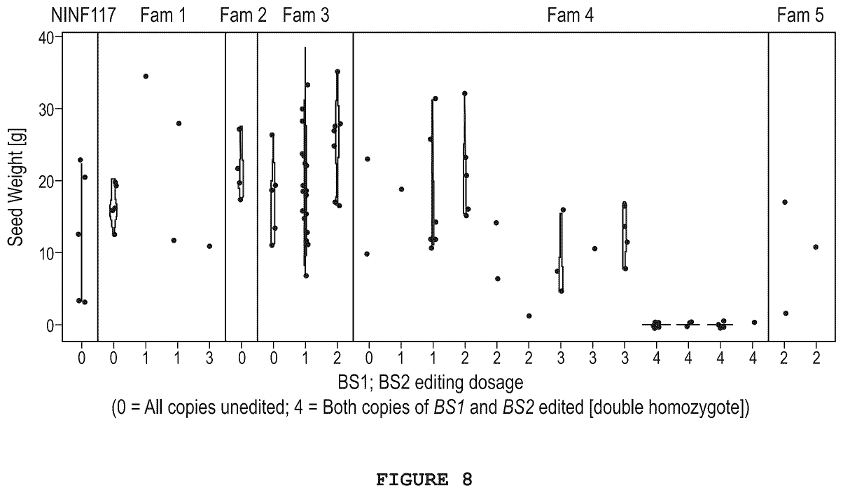

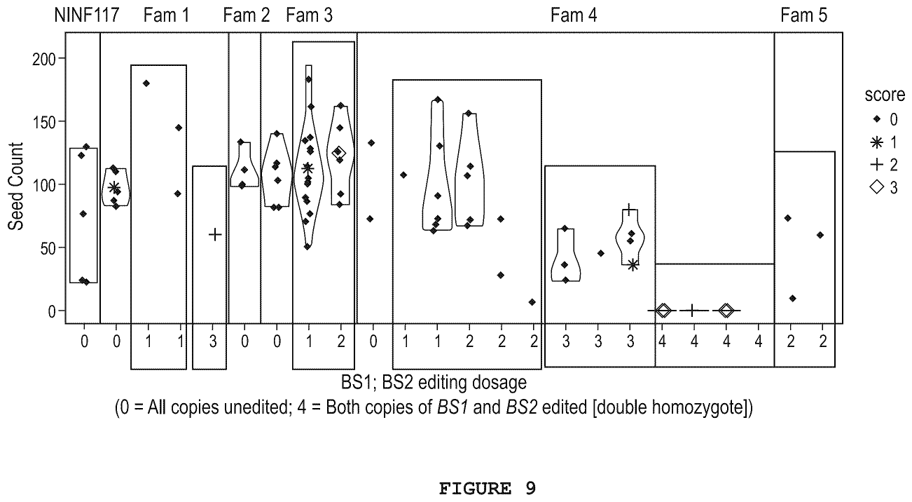

T1 soybean plants collected from T0 plants described in Example 2 were designated as families (Fam1, Fam2, Fam3, Fam4, and Fam5), and their harvested seed weight was analyzed (FIG. 8). It was found that higher BS1 and BS2 editing dosages correlated with reduced seed counts as shown in FIG. 9.

### Example 9. Analysis of mBS1 T1 Family 3 in Field

120 T1 seeds were planted from T1 Family 3 and resultant plants were assayed for presence of the transgene and edits in the BS1 or BS2 gene. Selected transgene null plants were analyzed and only the −1:5D BS2 deletion allele (SEQ ID NO: 31) was transmitted. 15 transgene null plants which were heterozygous for the −1:5D BS2 deletion allele (SEQ ID NO: 31) and 7 transgene null plants homozygous for the −1:5D BS2 deletion allele (SEQ ID NO: 31) were identified.

Seeds from these 7 T1 −1:5D BS2 deletion allele (SEQ ID NO: 32) homozygous edited plants were collected and planted in the field. Unedited NINF1170 and null NINF1170 soybean plants were used as checks (i.e., controls). Planting was at 30″ spacing; 15 ft single rows, with a target plant per acre (PPA) of about 110,000. Most phenotypes were on a plot basis. For intensive phenotyping, 2 ft of plants from a row (about 10 plants) were used.

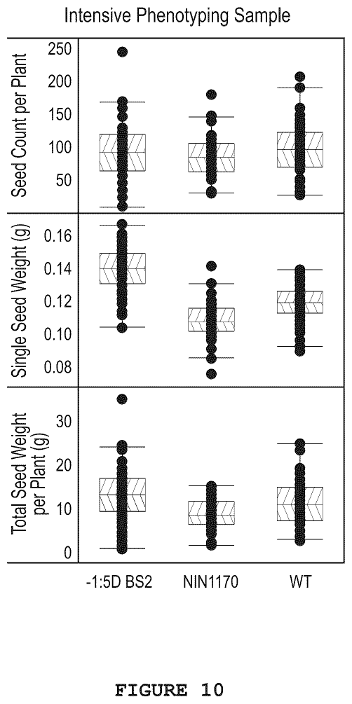

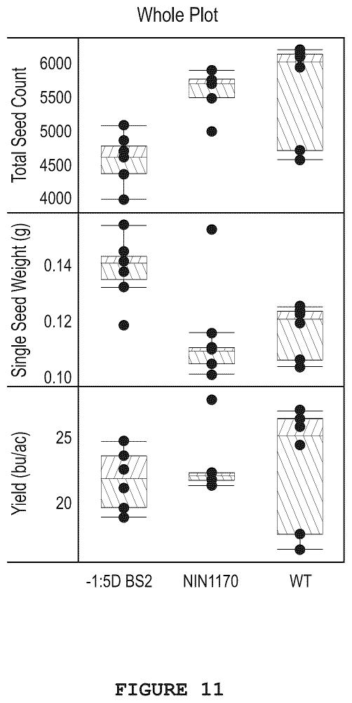

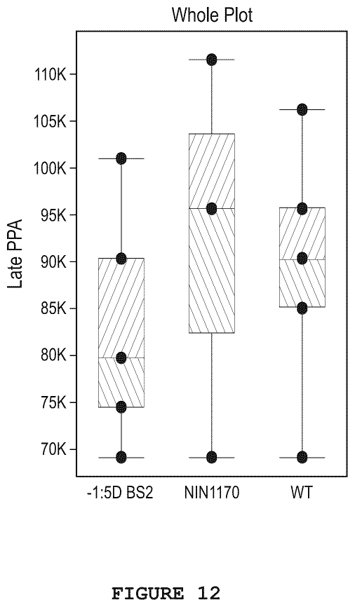

Data was collected at the plot level for agronomic phenotypes (yield, plant height, maturity date, PPA, plot wt., moisture, lodging, etc.); 1000 seed weight (5 subsamples), and seeds/lb (calculated from 1000 seed weight). Data was collected at a plant level for pod count per plant, seed count per plant, total weight of seeds per plant, and single seed weight (average per plant). Data for the field experiment are summarized in Table 13 and FIGS. 10, 11, and 12.

TABLE 13
Summary of field test data

| | AVERAGE | | | | | |
|---|---|---|---|---|---|---|
| | WT | AVERAGE | AVERAGE | AVERAGE | AVERAGE | AVERAGE |
| Allele | 1000 Seed | Seeds/lb | Pod Count | Seed Count | Seed Wt | Sg Seed Wt |
| WT, WT | 126.3 | 3606 | 48.4 | 95.3 | 11.3 | 0.1165 |
| (NINF1170 null) | | | | | | |
| Bs2_-1:5D | 148.9 | 3052 | 44.9 | 92.4 | 12.8 | 0.1383 |
| WT (NINF1170) | 119.1 | 3811 | 40.5 | 82.5 | 9.0 | 0.1081 |

The transgene null plants homozygous for the −1:5D BS2 deletion allele (SEQ ID NO: 31) exhibited increases in the average weight of 1000 seed, average seed weight, and average of single seed weight in comparison the wild-type checks. The transgene null plants homozygous for the −1:5D BS2 deletion allele (SEQ ID NO: 31) also exhibited a decrease in the average number of seeds per pound of seeds in comparison the wild-type checks.

### Example 10. Field Testing

A homozygous mutant for the −1:5D BS2 allele (SENF2104; SEQ ID NO: 31) was grown out in the field alongside null segregants (SENF2104N) and non-edited (wild-type) plants of the same genetic background (NINF1170). The weight per 100 seeds averaged from multiple field trials is shown in Table 14.

TABLE 14
Weight per 100 Seeds, Averaged from Field Trials

| | Avg. Weight/100 seeds (g) |
|---|---|
| NINF1170 | 16.97 |
| SENF2104 | 19.76 |
| SENF2104N | 17.15 |

Field plots were planted at eleven locations in Iowa, Ohio, Kansas, Nebraska, Indiana, and Illinois. The results consistently indicated, as shown in Table 15, that double BS2 null mutants (SENF2104) had seeds of higher weight as compared to wild-type plants (NINF1170) and null segregants (SENF2104N).

TABLE 15
Weight per 100 Seeds for Each Field Trial Location

Avg. Weight/100 seeds (g)

Location 1

| | |
|---|---|
| NINF1170 | 16.8 |
| SENF2104 | 20.6 |
| SENF2104N | 17.8 |

Location 2

| | |
|---|---|
| NINF1170 | 16.5 |
| SENF2104 | 19.6 |
| SENF2104N | 17.4 |

Location 3

| | |
|---|---|
| NINF1170 | 16.3 |
| SENF2104 | 18.2 |
| SENF2104N | 16.6 |

Location 4

| | |
|---|---|
| NINF1170 | 15.4 |
| SENF2104 | 17.6 |
| SENF2104N | 15.8 |

Location 5

| | |
|---|---|
| NINF1170 | 15.9 |
| SENF2104 | 18.6 |
| SENF2104N | 15.9 |

Location 6

| | |
|---|---|
| NINF1170 | 18.2 |
| SENF2104 | 21.6 |
| SENF2104N | 18.4 |

Location 7

| | |
|---|---|
| NINF1170 | 16.6 |
| SENF2104 | 20.1 |
| SENF2104N | 17.2 |

Location 8

| | |
|---|---|
| NINF1170 | 17.5 |
| SENF2104 | 20.5 |
| SENF2104N | 17.9 |

Location 9

| | |
|---|---|
| NINF1170 | 17.5 |
| SENF2104 | 20.7 |
| SENF2104N | 18.0 |

Location 10

| | |
|---|---|
| NINF1170 | 17.7 |
| SENF2104 | 19.0 |
| SENF2104N | 16.9 |

Location 11

| | |
|---|---|
| NINF1170 | 17.4 |
| SENF2104 | 20.5 |
| SENF2104N | 18.1 |

### DESCRIPTION OF BIOLOGICAL SEQUENCES

A description of the biological sequences which are provided in the co-filed sequence listing and also referred to in column 3 of the accompanying Tables 2 and 3 are provided in Table 16.

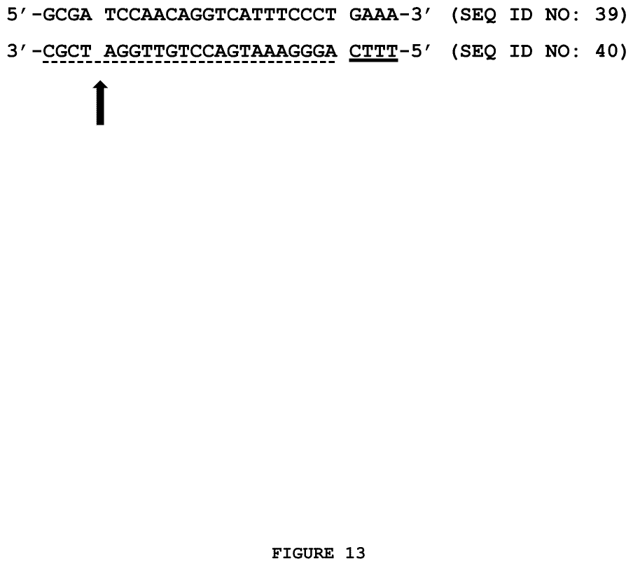

TABLE 16
Biological Sequences

| | SEQ | Sequence Type |
|---|---|---|
| | ID | DNA/PRT/ |
| Description of Sequence | NO | RNA |
| FT1a Protein (Glyma.18G298900.1; synonym: | 1 | PRT |
| Glyma18g53680; PEBP | | |
| (phosphatidylethanolamine-binding protein) | | |
| family protein)) | | |
| FT1a Protein (Glyma.18G298900.3; alternative | 2 | PRT |
| splicing form) | | |
| FT1a Genomic DNA (Glyma.18G298900.1 | 3 | DNA |
| (synonym: Glyma18g53680; PEBP | | |
| (phosphatidylethanolamine-binding protein) | | |
| family protein)); reference sequence for deletions | | |
| presented in column 3 of Table 2. | | |
| FT1a −10_13D Fragment | 4 | DNA |
| FT1a −10_13D FULL LENGTH | 5 | DNA |
| FT1a −10_13D encoded 84aa protein (splice | 6 | PRT |
| form 1 and 2) | | |
| FT1a −4_9D Fragment | 7 | DNA |
| FT1a −4_9D Full length | 8 | DNA |
| FT1a −4_9D encoded protein (splice form 1) | 9 | PRT |
| FT1a −4_9D encoded protein (splice form 2) | 10 | PRT |
| FT1a_KO_r1_T55: (DNA encoding spacer | 11 | DNA |
| sequence for gRNA used to make deletions) | | |
| FT1a WT segment (FIG. 5 and 6) | 12 | DNA |
| FT1a −16:41D (FIG. 5 and 6) | 13 | DNA |
| Jag1 WT segment (FIG. 5 and 6) | 14 | DNA |
| jag1 −9:13D segment with deletion (FIG. 5) | 15 | DNA |
| jag1 1:6D segment with deletion (FIG. 6) | 16 | DNA |
| JAG1_g3 (DNA encoding spacer sequence for | 17 | DNA |
| gRNA use) | | |
| Wild type JAG1 genomic DNA; reference | 18 | DNA |
| sequence for deletions presented in column 3 of | | |
| Table 3. | | |
| JAG1 genomic DNA with −9:13D deletion | 19 | DNA |
| JAG1 genomic DNA with 1:6D deletion | 20 | DNA |
| FT1a Genomic DNA with −16:41D deletion | 21 | DNA |
| TFL1b genomic DNA | 22 | DNA |
| TFL1b CDS | 23 | DNA |
| TFL1b | 24 | PRT |
| soybean BS1 gene (CH10), WT, Glyma10g38970 | 25 | DNA |
| soybean BS2 gene, WT | 26 | DNA |
| soybean BS1 protein, WT | 27 | PRT |
| soybean BS2 protein, WT | 28 | PRT |
| soybean BS2 protein, truncated −1:5D | 29 | PRT |
| soybean BS2 protein, truncated; 2:4D | 30 | PRT |
| soybean BS2 gene (CH20), amorphic | 31 | DNA |
| allele, −1:5D | | |
| soybean BS1 gene (CH10), amorphic | 32 | DNA |
| allele, −3:4D | | |
| soybean BS2 gene (CH20), amorphic | 33 | DNA |
| allele, −11:14D | | |
| soybean BS2 gene (CH20), amorphic | 34 | DNA |
| allele, −6:26D | | |
| soybean BS2 gene (CH20), amorphic | 35 | DNA |
| allele, 2:4D | | |
| BS1 _r2_T1 spacer RNA | 36 | DNA |
| (Cas12a Nuclease) | 37 | PRT |
| (>sp\|U2UMQ6\|CS12A_ACISB CRISPR- | | |
| associated endonuclease Cas12a | | |
| OS = Acidaminococcus sp. (strain BV3L6) | | |
| OX = 1111120 GN = cas12a PE = 1 SV = 1) | | |
| BS2 −1:5D junction | 38 | DNA |
| FIG. 13 BS1/BS2 guide RNA recog. site-sense | 39 | DNA |
| strand | | |
| FIG. 13 BS1/BS2 guide RNA recog. site- | 40 | DNA |
| antisense strand | | |

All cited patents and patent publications referred to in this application are incorporated herein by reference in their entirety. All of the materials and methods disclosed and claimed herein can be made and used without undue experimentation as instructed by the above disclosure and illustrated by the examples. Although the materials and methods of this disclosure have been described in terms of embodiments and illustrative examples, it will be apparent to those of skill in the art that substitutions and variations can be applied to the materials and methods described herein without departing from the concept, spirit, and scope of the disclosure. All such similar substitutes and modifications apparent to those skilled in the art are deemed to be within the spirit, scope, and concept of the disclosure as encompassed by the embodiments of the disclosures recited herein and the specification and appended claims.

What is claimed is:

1. A soybean plant cell comprising a loss-of-function allele of the endogenous soybean FT1a gene of SEQ ID NO: 3 or an allelic variant thereof.

2. The soybean plant cell of claim 1, wherein the plant cell is homozygous for the loss-of-function allele of the soybean FT1a gene of SEQ ID NO: 3 or allelic variant thereof.

3. The soybean plant cell of claim 1, wherein the loss-of-function allele comprises an amorphic allele of the FT1a gene of SEQ ID NO: 3 or allelic variant thereof.

4. The soybean plant cell of claim 1, wherein the loss-of-function allele comprises a hypomorphic allele of the FT1a gene.

5. The soybean plant cell of claim 1, wherein the loss-of-function allele comprises a frameshift mutation or a nonsense mutation in the coding region of the FT1a gene of SEQ ID NO: 3 or allelic variant thereof.

6. The soybean plant cell of claim 5, wherein the loss-of-function allele comprises a frameshift mutation or a nonsense mutation in the first exon of the FT1a gene of SEQ ID NO: 3 or allelic variant thereof; optionally wherein the frameshift or nonsense mutation occurs at nucleotides corresponding to nucleotides 340 to 343 of SEQ ID NO: 3 or an allelic variant thereof.

7. The soybean plant cell of claim 6, wherein the loss-of-function allele comprises the frameshift mutation of SEQ ID NO: 4 or SEQ ID NO: 5 or wherein the ft1a gene comprising the frameshift mutation encodes the protein of SEQ ID NO: 6.

8. The soybean plant cell of claim 1, wherein the loss-of-function allele comprises an internal deletion comprising: (i) at least nucleotides corresponding to nucleotides 318 to 345, 319 to 356, 320 to 352, 321 to 348, 322 to 350, 323 to 328, 324 to 328, 325 to 347, 326 to 349, 327 to 347, 328 to 348, 329 to 347, 330 to 348, 331 to 347, 332 to 347, 333 to 347, 334 to 345, 335 to 347, 336 to 347, 337 to 347, 338 to 345, 339 to 345, 340, 341 to 345, 342 to 344, 343 to 347, 343 to 351,344 to 345, 345 to 347, 346 to 347, 347 to 350, 348 to 351, or 331 to 371 of SEQ ID NO: 3 or an allelic variant thereof; or (ii) at least nucleotides encoding amino acids corresponding to N39 to C41 of SEQ ID NO: 1 or SEQ ID NO: 2; optionally in either (i) or (ii) wherein the internal deletion preserves the reading frame of the encoded ft1a mutant protein comprising the loss-of-function allele with respect to amino acid residues of the ft1a mutant protein which have not been deleted.

9. The soybean plant cell of claim 8, wherein the internal deletion comprises: (i) the internal deletion of SEQ ID NO: 7 or SEQ ID NO: 8; or (ii) the internal deletion which encodes a protein corresponding to the protein of SEQ ID NO: 9 and/or SEQ ID NO: 10.

10. The soybean plant cell of claim 1, wherein the soybean plant cell further comprises: (i) one or more mutations in a distinct soybean gene or genes; and/or (ii) one or more transgenes, optionally wherein said transgenes encode proteins or RNAs conferring herbicide tolerance or pest tolerance.

11. The soybean plant cell of claim 1, wherein the soybean plant cell comprises elite soybean germplasm.

12. The soybean plant cell of claim 10, wherein the one or more mutations in a distinct soybean gene or genes comprise:
(i) a loss-of-function allele of an endogenous soybean JAG1 gene of SEQ ID NO: 18 or an allelic variant thereof, optionally wherein the loss-of-function allele comprises an internal deletion comprising at least nucleotides corresponding to nucleotides 408 to 426, 409 to 427, 410 to 429, 411 to 428, 412 to 428, 413 to 427, 414 to 426, 415 to 427, 416 to 427, 417 to 426, 418 to 426, 419 to 426, 420 to 426, 421 to 426, 422 to 426, 423 to 426, 424 to 427, 425 to 427, 425 to 431, 426 to 429, 427 to 428, 429 to 430, 430, or 431 of SEQ ID NO: 18 or an allelic variant thereof;
(ii) a loss-of-function allele of an endogenous soybean BS1 gene of SEQ ID NO: 25 or an allelic variant thereof;
(iii) a loss-of-function allele of an endogenous soybean BS2 gene of SEQ ID NO: 26 or an allelic variant thereof, and/or
(iv) a loss-of function mutation in an endogenous soybean TFL1b gene of SEQ ID NO: 22 or an allelic variant thereof.

13. A soybean plant part comprising the soybean plant cell of claim 1.

14. The soybean plant part of claim 13, wherein the part is a stem, root, leaf, flower, pod, or seed.

15. The soybean plant part of claim 14, wherein the part is a seed.

16. The soybean plant part of claim 14, wherein the part is a seed and wherein the total harvested seed weight per acre or hectare of a population of soybean plants grown from the seed is increased in comparison to total harvested seed weight per acre or hectare of a population of wild-type control soybean plants lacking the loss-of-function allele.

17. The soybean plant part of claim 14, wherein the part is a seed and wherein the average weight of the seed is equivalent to the average weight of a wild-type control seed lacking the loss-of-function allele.

18. The soybean plant part of claim 14, wherein the part is a pod; optionally wherein pod count for a soybean plant which is homozygous for the loss-of-function mutation and from which the pod was obtained is increased in comparison to pod count of a wild-type control plant lacking the loss-of-function allele.

19. A soybean plant comprising the soybean plant cell of claim 1.

20. The soybean plant of claim 19, wherein pod count per plant; seed count per plant; and/or total harvested seed weight per plant are increased in comparison to pod count per plant; seed count per plant; and/or total harvested seed weight per plant for a wild-type control soybean plant lacking the loss-of-function allele.

21. The soybean plant of claim 19, wherein pod count per plant; seed count per plant; and/or total harvested seed weight per plant are increased when the plant is grown under stress in comparison to pod count per plant; seed count per plant; and/or total harvested seed weight per plant for a wild-type control soybean plant lacking the loss-of-function allele grown under stress, optionally wherein the stress comprises drought stress.
